(19) United States
(12) Patent Application Publication
(10) Pub. No.: US 2024/0409860 A1
(43) Pub. Date: Dec. 12, 2024

(54) METHODS AND COMPOSITIONS FOR REDUCING SMOKE TAINT IN FERMENTED BEVERAGES

(71) Applicant: Berkeley Fermentation Science Inc., Oakland, CA (US)

(72) Inventors: Rachel Li, Berkeley, CA (US); Nicholas Harris, Glenwood Springs, CO (US); Charles Denby, Berkeley, CA (US); Jeremy Roop, Berkeley, CA (US); Daniel Liu, San Mateo, CA (US); Francis Cunningham, Oakland, CA (US)

(73) Assignee: Berkeley Fermentation Science Inc., Oakland, CA (US)

(21) Appl. No.: 18/699,011

(22) PCT Filed: Oct. 7, 2022
(86) PCT No.: PCT/US2022/077806

§ 371 Date: Apr. 5, 2024

Related U.S. Application Data
(60) Provisional application No. 63/254,042, filed on Oct. 8, 2021.

(51) Int. Cl. C12G 1/022; C12H 1/00; C12H 1/044; C12H 1/056; C12H 1/07; C12N 9/10; C12N 9/24; C12N 9/42

(52) U.S. Cl. CPC C12G 1/0203; C12H 1/003; C12H 1/0408; C12H 1/0424; C12H 1/063; C12N 9/1007; C12N 9/2402; C12N 9/2445; C12Y 302/01021; C12Y 302/0104; C12G 2200/05; C12G 2200/11; C12G 2200/15

(57) ABSTRACT

Provided herein are genetically modified yeast cells that recombinantly express a glycosidase enzyme. Provided herein are genetically modified yeast cells that recombinantly express a glycosidase enzyme and/or an O-methyltransferase enzyme. Also provided are methods of producing fermented beverages and compositions comprising ethanol using the genetically modified yeast cells described herein.

### CROSS-REFERENCE TO RELATED APPLICATIONS

This application claims the benefit under 35 U.S.C. § 119(e) of U.S. Provisional Application No. 63/254,042, filed Oct. 8, 2021, entitled “METHODS AND COMPOSITIONS FOR REDUCING SMOKE TAINT IN FERMENTED BEVERAGES,” the entire disclosure of which is hereby incorporated by reference in its entirety.

### REFERENCE TO AN ELECTRONIC SEQUENCE LISTING

The contents of the electronic sequence listing (B150970005WO00-SEQ-CEW.xml; Size: 77,000 bytes; and Date of Creation: Oct. 6, 2022) is herein incorporated by reference in its entirety.

### BACKGROUND

The exposure of grapes and hops to smoke can result in smoke-tainted wines and beers produced from exposed grapes and hops, respectively, with undesirable sensory notes such as wood smoke, ash, medicinal, and barbecued meat. This phenomenon, termed smoke taint, has become a major issue for winemakers—and more recently brewers—as the frequency and duration of wildfires has increased in recent years. Reports of financial losses to the wine industry vary, but are estimated to be in the billions of dollars annually due to unharvested grapes, un-made wine, or wine deemed unfit for sale. Damage from the 2020 wildfires alone may have cost California's wine industry as much as $3.7 billion (Mobley, E. “Without federal aid for wildfires, California's wine industry could collapse vintners say” San Francisco Chronicle. (Sep. 23, 2021)).

### SUMMARY

The present disclosure relates, at least in part, to genetically modified yeast cells capable of reducing volatile phenols, such as volatile phenols associated with smoke taint, in a fermented product, and methods of use thereof in producing fermented beverages, such as beer, wine, and spirits, and compositions comprising ethanol.

Aspects of the present disclosure relate to methods of producing a fermented product comprising contacting a genetically modified yeast cell (modified cell) with a medium comprising at least one fermentable sugar and one or more non-volatile phenolic glycosides associated with smoke taint, thereby hydrolyzing the one or more non-volatile phenolic glycosides to produce one or more volatile phenols; wherein the genetically modified cell comprises a heterologous gene encoding an enzyme having glycosidase activity.

In some embodiments, the contacting is performed during at least a first fermentation process to produce a first fermented product comprising volatile phenols. In some embodiments, the method further comprises removing one or more volatile phenols from the first fermented product to produce a second fermented product. In some embodiments, the enzyme having glycosidase activity is a glucosidase or a rhamnosidase. In some embodiments, the glucosidase is a beta-D-glucosidase or the rhamnosidase is an alpha-L-rhamnosidase.

In some embodiments, the enzyme having glycosidase activity is derived from Aspergillus aculatus, Aspergillus aculatus, Aspergillus terreus, Aspergillus nidulans, Aspergillus niger, Aspergillus oryzae, Lactobacillus plantarum, Lactobacillus acidophilus, Thermomicrobia PRI-1686, Candida molischiana, Bacillus polymyxa, Olea europaea, Saccharomyces cerevisiae, Thermoascus aurantiacus, Aspergillus niger, Trichoderma reesei, Neurospora crassa, Oenococcus oeni, Prunis dulcis, Bacillus circulans, or Sinorhizobium meliloti. In some embodiments, the enzyme having glycosidase activity is selected from the group consisting of RhaB derived from Aspergillus aculatus, RhaA derived from Aspergillus aculatus, AtRha derived from Aspergillus terreus, AndRha derived from Aspergillus nidulans, AngRha derived from Aspergillus niger, AorhaA derived from Aspergillus oryzae, RhaB1 derived from Lactobacillus plantarum NCC245, RhaB2 derived from Lactobacillus plantarum NCC245, LpRam1 derived from Lactobacillus acidophilus, RhmA derived from Thermomicrobia PRI-1686, RhmB derived from Thermomicrobia PRI-1686, bgln derived from Candida molischiana, bglA derived from Bacillus polymyxa, OepGLU derived from Olea europaea, EGH1 derived from Saccharomyces cerevisiae, Tabgl1 derived from Thermoascus aurantiacus IF09748, AoBGL1 derived from Aspergillus oryzae, AnBGL1 derived from Aspergillus niger, TrBGL2 derived from Trichoderma reesei, TrBGL1 derived from Trichoderma reesei, BGL1 derived from Neurospora crassa, BGL2 derived from Neurospora crassa, bglH derived from Aspergillus oryzae, OoBgl derived from Oenococcus oeni, Ph691 from Prunus dulcis, Ph692 from Prunus dulcis, BsBglA from Bacillus circulans, or SmBgl from Sinorhizobium meliloti.

In some embodiments, the enzyme having glycosidase activity comprises a sequence having at least 90% sequence identity to the amino acid sequences set forth in any of SEQ ID NOs: 1-28. In some embodiments, the enzyme having glycosidase activity comprises the amino acid sequence set forth in any of SEQ ID NOs: 1-28. In some embodiments, the enzyme having glycosidase activity comprises a secretion signal. In some embodiments, the secretion signal is a peptide selected from the group consisting of SED1, MATα, MATα pre-sequence, TFP5-1, TFP1-4, TFP10, TFP23, SUC2, SRL1, and KSH1. In some embodiments, the signal sequence comprises the amino acid sequence set forth in any of SEQ ID NOs: 29-36 and 50-54.

In some embodiments, removing one or more volatile phenols from the first fermented product comprises filtering the first fermented product. In some embodiments, the filtering comprises subjecting the first fermented product to reverse osmosis. In some embodiments, removing one or more volatile phenols from the first fermented product comprises contacting the first fermented product with a fining agent. In some embodiments, the fining agent is activated carbon or a cyclodextrin polymers. In some embodiments, removing one or more volatile phenols from the first fermented product comprises contacting the first fermented product with an enzyme having O-methyltransferase activity. In some embodiments, the enzyme having O-methyltransferase activity is a recombinant, purified O-methyl transferase enzyme. In some embodiments, the recombinant, purified O-methyl transferase enzyme is added to the first fermented product. In some embodiments, the method further comprises adding S-adenosyl methionine to the medium or the first fermented product.

In some embodiments, the modified cell further comprises a second heterologous gene encoding an enzyme having O-methyltransferase activity; and the removing one or more volatile phenols from the first fermented product comprises producing one or more methylated volatile phenols (also referred to as methyl ethers). In some embodiments, the second heterologous gene encoding an enzyme having O-methyltransferase activity is derived from Silene latifolia, Solanuma lycopersicum, Rosa hybrida, Ocimum basilicum, Eriobotrya japonica, Pinus taeda, Fragaria ananassa, Schizosaccharomyces pombe, or Zinnia elegans.

In some embodiments, the enzyme having O-methyltransferase activity is selected from the group consisting of SIGOMT4 derived from Silene latifolia, SIOMT1 derived from Solanuma lycopersicum, SIOMT4 derived from Solanuma lycopersicum, SICTOMT1 derived from Solanuma lycopersicum, RhOOMT1 derived from Rosa hybrida, RhOOMT2 derived from Rosa hybrida, EOMT1 derived from Ocimum basilicum, EjOMT1 derived from Eriobotrya japonica, AEOMT derived from Pinus taeda, FaOMT derived from Fragaria ananassa, SpCOMT derived from Schizosaccharomyces pombe, COMT1 derived from Ocimum basilicum, and ZeCAOMT derived from Zinnia elegans.

In some embodiments, the enzyme having O-methyltransferase activity comprises a sequence having at least 90% sequence identity to the amino acid sequences set forth in any of SEQ ID NOs: 37-49. In some embodiments, the enzyme having O-methyltransferase activity comprises the amino acid sequence set forth in any of SEQ ID NOs: 37-49.

In some embodiments, the one or more non-volatile phenolic glycoside associated with smoke taint comprises a glucoside, a gentiobioside, and/or a rutinoside. In some embodiments, one or more volatile phenol comprises guaiacol, m-cresol, p-cresol, o-cresol, phenol, 4-methylguaiacol, syringol, and/or 4-methylsyringol. In some embodiments, the one or more methylated volatile phenols comprises veratrole (1,2-dimethoxybenzene) and/or 3-methylanisole.

In some embodiments, the yeast cell is of the genus Saccharomyces. In some embodiments, the yeast cell is of the species Saccharomyces cerevisiae (S. cerevisiae). In some embodiments, the yeast cell is S. cerevisiae California Ale Yeast strain WLP001, EC-1118, Elegance, Red Star Côte des Blancs, Epernay II, London Ale III, Augustiner, W-34/70, Andechs, D254, RC212, or B0213. In some embodiments, the yeast cell is of the species Saccharomyces pastorianus (S. pastorianus).

In some embodiments, at least one fermentable sugar is provided in at least one sugar source. In some embodiments, the fermentable sugar is glucose, fructose, sucrose, maltose, and/or maltotriose. In some embodiments, the fermented product comprises a reduced level of one or more non-volatile phenolic glycosides as compared to a fermented product produced by a counterpart cell that does not express the heterologous gene. In some embodiments, the fermented product is a fermented beverage. In some embodiments, the fermented beverage is beer, wine, sparkling wine (champagne), wine cooler, wine spritzer, hard seltzer, sake, mead, kombucha, or cider.

In some embodiments, the sugar source comprises wort, must, fruit juice, honey, rice starch, or a combination thereof. In some embodiments, the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit. In some embodiments, the sugar source is wort and the method further comprises producing the medium, wherein producing the medium comprises: (a) contacting a plurality of grains with water; and (b) boiling or steeping the water and grains to produce wort. In some embodiments, the method further comprises adding at least one hop variety to the wort to produce a hopped wort. In some embodiments, the method further comprises adding at least one hop variety to the medium. In some embodiments, the sugar source is must and the method further comprises producing the medium, wherein producing the medium comprises crushing a plurality of fruit to produce the must. In some embodiments, the method further comprises removing solid fruit material from the must to produce a fruit juice. In some embodiments, the method further comprises at least one additional fermentation process. In some embodiments, the method further comprises carbonating the fermented product.

Another aspect of the present disclosure relates to a genetically modified yeast cell (modified cell) comprising (i) a first heterologous gene encoding an enzyme having glycosidase activity, and (ii) a second heterologous gene encoding an enzyme having O-methyltransferase activity.

In some embodiments, the enzyme having glycosidase activity is a glucosidase or a rhamnosidase. In some embodiments, the glucosidase is a beta-D-glucosidase or the rhamnosidase is an alpha-L-rhamnosidase.

In some embodiments, the enzyme having glycosidase activity is derived from Aspergillus aculatus, Aspergillus aculatus, Aspergillus terreus, Aspergillus nidulans, Aspergillus niger, Aspergillus oryzae, Lactobacillus plantarum, Lactobacillus acidophilus, Thermomicrobia PRI-1686, Candida molischiana, Bacillus polymyxa, Olea europaea, Saccharomyces cerevisiae, Thermoascus aurantiacus, Aspergillus niger, Trichoderma reesei, Neurospora crassa, Oenococcus oeni, Prunis dulcis, Bacillus circulans, or Sinorhizobium meliloti. In some embodiments, the enzyme having glycosidase activity is selected from the group consisting of RhaB derived from Aspergillus aculatus, RhaA derived from Aspergillus aculatus, AtRha derived from Aspergillus terreus, AndRha derived from Aspergillus nidulans, AngRha derived from Aspergillus niger, AorhaA derived from Aspergillus oryzae, RhaB1 derived from Lactobacillus plantarum NCC245, RhaB2 derived from Lactobacillus plantarum NCC245, LpRam1 derived from Lactobacillus acidophilus, RhmA derived from Thermomicrobia PRI-1686, RhmB derived from Thermomicrobia PRI-1686, bgln derived from Candida molischiana, bglA derived from Bacillus polymyxa, OepGLU derived from Olea europaea, EGH1 derived from Saccharomyces cerevisiae, Tabgl1 derived from Thermoascus aurantiacus IF09748, AoBGL1 derived from Aspergillus oryzae, AnBGL1 derived from Aspergillus niger, TrBGL2 derived from Trichoderma reesei, TrBGL1 derived from Trichoderma reesei, BGL1 derived from Neurospora crassa, BGL2 derived from Neurospora crassa, bglH derived from Aspergillus oryzae, OoBgl derived from Oenococcus oeni, Ph691 from Prunus dulcis, Ph692 from Prunus dulcis, BsBglA from Bacillus circulans, or SmBgl from Sinorhizobium meliloti.

In some embodiments, the enzyme having glycosidase activity comprises a sequence having at least 90% sequence identity to the amino acid sequences set forth in any of SEQ ID NOs: 1-28. In some embodiments, the enzyme having glycosidase activity comprises the amino acid sequence set forth in any of SEQ ID NOs: 1-28.

In some embodiments, the enzyme having glycosidase activity comprises a secretion signal. In some embodiments, the secretion signal is a peptide selected from the group consisting of SED1, MATα, MATα pre-sequence, TFP5-1, TFP1-4, TFP10, TFP23, SUC2, SRL1, and KSH1. In some embodiments, the signal sequence comprises the amino acid sequence set forth in any of SEQ ID NOs: 29-36 and 50-54.

In some embodiments, the second heterologous gene encoding an enzyme having O-methyltransferase activity is derived from Silene latifolia, Solanuma lycopersicum, Rosa hybrida, Ocimum basilicum, Eriobotrya japonica, Pinus taeda, Fragaria ananassa, Schizosaccharomyces pombe, or Zinnia elegans. In some embodiments, the enzyme having O-methyltransferase activity is selected from the group consisting of SIGOMT4 derived from Silene latifolia, SIOMT1 derived from Solanuma lycopersicum, SIOMT4 derived from Solanuma lycopersicum, SICTOMT1 derived from Solanuma lycopersicum, RhOOMT1 derived from Rosa hybrida, RhOOMT2 derived from Rosa hybrida, EOMT1 derived from Ocimum basilicum, EjOMT1 derived from Eriobotrya japonica, AEOMT derived from Pinus taeda, FaOMT derived from Fragaria ananassa, SpCOMT derived from Schizosaccharomyces pombe, COMT1 derived from Ocimum basilicum, and ZeCAOMT derived from Zinnia elegans.

In some embodiments, the enzyme having O-methyltransferase activity comprises a sequence having at least 90% sequence identity to the amino acid sequences set forth in any of SEQ ID NOs: 37-49. In some embodiments, the enzyme having O-methyltransferase activity comprises the amino acid sequence set forth in any of SEQ ID NOs: 37-49.

In some embodiments, the yeast cell is of the genus Saccharomyces. In some embodiments, the yeast cell is of the species Saccharomyces cerevisiae (S. cerevisiae). In some embodiments, the yeast cell is S. cerevisiae California Ale Yeast strain WLP001, EC-1118, Elegance, Red Star Côte des Blancs, Epernay II, London Ale III, Augustiner, W-34/70, Andechs, D254, RC212, or B0213. In some embodiments, the yeast cell is of the species Saccharomyces pastorianus (S. pastorianus).

Aspects of the present disclosure relate to methods of producing a fermented product comprising, contacting any of the modified cells described herein with a medium comprising at least one fermentable sugar, wherein the contacting is performed during at least a first fermentation process, to produce a fermented product.

In some embodiments, the medium comprises one or more non-volatile phenolic glycoside associated with smoke taint. In some embodiments, the non-volatile phenolic glycoside associated with smoke taint comprises a glucoside, a gentiobioside, and/or a rutinoside. In some embodiments, the fermented product comprises one or more methylated volatile phenols. In some embodiments, the one or more methylated volatile phenols comprises veratrole (1,2-dimethoxybenzene), 4-methylveratrole, 2-methylanisole, 3-methylanisole, and/or 4-methylanisole.

In some embodiments, at least one fermentable sugar is provided in at least one sugar source. In some embodiments, the fermentable sugar is glucose, fructose, sucrose, maltose, and/or maltotriose.

In some embodiments, the fermented product comprises a reduced level of one or more non-volatile phenolic glycosides as compared to a fermented product produced by a counterpart cell that does not express the first heterologous gene and/or second heterologous gene. In some embodiments, the fermented product is a fermented beverage. In some embodiments, the fermented beverage is beer, wine, sparkling wine (champagne), wine cooler, wine spritzer, hard seltzer, sake, mead, kombucha, or cider.

In some embodiments, the sugar source comprises wort, must, fruit juice, honey, rice starch, or a combination thereof. In some embodiments, the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

In some embodiments, the sugar source is wort and the method further comprises producing the medium, wherein producing the medium comprises (a) contacting a plurality of grains with water; and (b) boiling or steeping the water and grains to produce wort.

In some embodiments, the method further comprises adding at least one hop variety to the wort to produce a hopped wort. In some embodiments, the method further comprises adding at least one hop variety to the medium.

In some embodiments, the sugar source is must and the method further comprises producing the medium, wherein producing the medium comprises crushing a plurality of fruit to produce the must. In some embodiments, the method further comprises removing solid fruit material from the must to produce a fruit juice.

In some embodiments, the method further comprises at least one additional fermentation process. In some embodiments, the method further comprises carbonating the fermented product.

Aspects of the present disclosure relate to methods of producing a composition comprising ethanol comprising, contacting the modified cell with a medium comprising at least one fermentable sugar, wherein the contacting is performed during at least a first fermentation process, to produce a composition comprising ethanol.

In some embodiments, the medium comprises one or more non-volatile phenolic glycosides associated with smoke taint. In some embodiments, the non-volatile phenolic glycoside associated with smoke taint comprises a glucoside, a gentiobioside, and/or a rutinoside. In some embodiments, wherein the composition further comprises one or more methylated volatile phenols. In some embodiments, the one or more methylated volatile phenols comprises veratrole (1,2-dimethoxybenzene), 4-methylveratrole, 2-methylanisole, 3-methylanisole, and/or 4-methylanisole.

In some embodiments, at least one fermentable sugar is provided in at least one sugar source. In some embodiments, the fermentable sugar is glucose, fructose, sucrose, maltose, and/or maltotriose.

In some embodiments, the composition comprises a reduced level of one or more non-volatile phenolic glycosides as compared to a composition produced by a counterpart cell that does not express the first heterologous gene and/or second heterologous gene. In some embodiments, the composition is a fermented beverage. In some embodiments, the fermented beverage is beer, wine, sparkling wine (champagne), wine cooler, wine spritzer, hard seltzer, sake, mead, kombucha, or cider.

In some embodiments, the sugar source comprises wort, must, fruit juice, honey, rice starch, or a combination thereof. In some embodiments, the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

In some embodiments, the sugar source is wort and the method further comprises producing the medium, wherein producing the medium comprises (a) contacting a plurality of grains with water; and (b) boiling or steeping the water and grains to produce wort. In some embodiments, the method further comprises adding at least one hop variety to the wort to produce a hopped wort. In some embodiments, the method further comprises adding at least one hop variety to the medium.

In some embodiments, the sugar source is must and the method further comprises producing the medium, wherein producing the medium comprises crushing a plurality of fruit to produce the must. In some embodiments, the method further comprises removing solid fruit material from the must to produce a fruit juice. In some embodiments, the method further comprises at least one additional fermentation process. In some embodiments, the method further comprises carbonating the composition.

Aspects of the present disclosure relate to a method comprising contacting a genetically modified yeast cell (modified cell) with a medium comprising at least one fermentable sugar and one or more volatile phenols, thereby converting the one or more volatile phenols to one or more methylated volatile phenols; wherein the genetically modified cell comprises a heterologous gene encoding an enzyme having O-methyltransferase activity.

In some embodiments, wherein the medium further comprises one or more non-volatile phenolic glycosides associated with smoke taint. In some embodiments, the method further comprises contacting the medium with an enzyme having glycosidase activity thereby producing the one or more volatile phenols.

In some embodiments, the enzyme having glycosidase activity is a recombinant and/or purified glycosidase enzyme. In some embodiments, the enzyme having glycosidase activity is a glucosidase or a rhamnosidase. In some embodiments, the glucosidase is a beta-D-glucosidase or the rhamnosidase is an alpha-L-rhamnosidase. In some embodiments, the enzyme having glycosidase activity is a recombinant and/or purified glycosidase enzyme obtained from almonds.

In some embodiments, the enzyme having glycosidase activity is derived from Aspergillus aculatus, Aspergillus aculatus, Aspergillus terreus, Aspergillus nidulans, Aspergillus niger, Aspergillus oryzae, Lactobacillus plantarum, Lactobacillus acidophilus, Thermomicrobia PRI-1686, Candida molischiana, Bacillus polymyxa, Olea europaea, Saccharomyces cerevisiae, Thermoascus aurantiacus, Aspergillus niger, Trichoderma reesei, Neurospora crassa, Oenococcus oeni, Prunis dulcis, Bacillus circulans, or Sinorhizobium meliloti. In some embodiments, the enzyme having glycosidase activity is derived from almond. In some embodiments, the enzyme having glycosidase activity is selected from the group consisting of RhaB derived from Aspergillus aculatus, RhaA derived from Aspergillus aculatus, AtRha derived from Aspergillus terreus, AndRha derived from Aspergillus nidulans, AngRha derived from Aspergillus niger, AorhaA derived from Aspergillus oryzae, RhaB1 derived from Lactobacillus plantarum NCC245, RhaB2 derived from Lactobacillus plantarum NCC245, LpRam1 derived from Lactobacillus acidophilus, RhmA derived from Thermomicrobia PRI-1686, RhmB derived from Thermomicrobia PRI-1686, bgln derived from Candida molischiana, bglA derived from Bacillus polymyxa, OepGLU derived from Olea europaea, EGH1 derived from Saccharomyces cerevisiae, Tabgl1 derived from Thermoascus aurantiacus IF09748, AoBGL1 derived from Aspergillus oryzae, AnBGL1 derived from Aspergillus niger, TrBGL2 derived from Trichoderma reesei, TrBGL1 derived from Trichoderma reesei, BGL1 derived from Neurospora crassa, BGL2 derived from Neurospora crassa, bglH derived from Aspergillus oryzae, OoBgl derived from Oenococcus oeni, Ph691 from Prunus dulcis, Ph692 from Prunus dulcis, BsBglA from Bacillus circulans, or SmBgl from Sinorhizobium meliloti.

In some embodiments, the enzyme having glycosidase activity comprises a sequence having at least 90% sequence identity to the amino acid sequences set forth in any of SEQ ID NOs: 1-28. In some embodiments, the enzyme having glycosidase activity comprises the amino acid sequence set forth in any of SEQ ID NOs: 1-28.

In some embodiments, the enzyme having O-methyltransferase activity is derived from Silene latifolia, Solanuma lycopersicum, Rosa hybrida, Ocimum basilicum, Eriobotrya japonica, Pinus taeda, Fragaria ananassa, Schizosaccharomyces pombe, or Zinnia elegans.

In some embodiments, the heterologous gene encoding an enzyme having O-methyltransferase activity is derived from Silene latifolia, Solanuma lycopersicum, Rosa hybrida, Ocimum basilicum, Eriobotrya japonica, Pinus taeda, Fragaria ananassa, Schizosaccharomyces pombe, or Zinnia elegans. In some embodiments, the enzyme having O-methyltransferase activity is selected from the group consisting of SIGOMT4 derived from Silene latifolia, SIOMT1 derived from Solanuma lycopersicum, SIOMT4 derived from Solanuma lycopersicum, SICTOMT1 derived from Solanuma lycopersicum, RhOOMT1 derived from Rosa hybrida, RhOOMT2 derived from Rosa hybrida, EOMT1 derived from Ocimum basilicum, EjOMT1 derived from Eriobotrya japonica, AEOMT derived from Pinus taeda, FaOMT derived from Fragaria ananassa, SpCOMT derived from Schizosaccharomyces pombe, COMT1 derived from Ocimum basilicum, and ZeCAOMT derived from Zinnia elegans.

In some embodiments, the enzyme having O-methyltransferase activity comprises a sequence having at least 90% sequence identity to the amino acid sequences set forth in any of SEQ ID NOs: 37-49. In some embodiments, the enzyme having O-methyltransferase activity comprises the amino acid sequence set forth in any of SEQ ID NOs: 37-49. In some embodiments, the enzyme having O-methyltransferase activity comprises a secretion signal. In some embodiments, the secretion signal is a peptide selected from the group consisting of SED1, MATα, MATα pre-sequence, TFP5-1, TFP1-4, TFP10, TFP23, SUC2, SRL1, and KSH1. In some embodiments, the signal sequence comprises the amino acid sequence set forth in any of SEQ ID NOs: 29-36 and 50-54.

In some embodiments, removing one or more volatile phenols from the first fermented product comprises filtering the first fermented product. In some embodiments, the filtering comprises subjecting the first fermented product to reverse osmosis. In some embodiments, removing one or more volatile phenols from the first fermented product comprises contacting the first fermented product with a fining agent. In some embodiments, the fining agent is activated carbon or a cyclodextrin polymers.

In some embodiments, the one or more non-volatile phenolic glycoside associated with smoke taint comprises a glucoside, a gentiobioside, and/or a rutinoside. In some embodiments, one or more volatile phenol comprises guaiacol, m-cresol, p-cresol, o-cresol, phenol, 4-methylguaiacol, syringol, and/or 4-methylsyringol. In some embodiments, the one or more methylated volatile phenols comprises veratrole (1,2-dimethoxybenzene), 4-methylveratrole, 2-methylanisole, 3-methylanisole, and/or 4-methylanisole.

In some embodiments, the yeast cell is of the genus Saccharomyces. In some embodiments, the yeast cell is of the species Saccharomyces cerevisiae (S. cerevisiae). In some embodiments, the yeast cell is S. cerevisiae California Ale Yeast strain WLP001, EC-1118, Elegance, Red Star Côte des Blancs, Epernay II, London Ale III, Augustiner, W-34/70, Andechs, D254, RC212, or B0213. In some embodiments, the yeast cell is of the species Saccharomyces pastorianus (S. pastorianus).

In some embodiments, at least one fermentable sugar is provided in at least one sugar source. In some embodiments, the fermentable sugar is glucose, fructose, sucrose, maltose, and/or maltotriose. In some embodiments, the fermented product comprises a reduced level of one or more non-volatile phenolic glycosides as compared to a fermented product produced by a counterpart cell that does not express the heterologous gene.

In some embodiments, the fermented product is a fermented beverage. In some embodiments, the fermented beverage is beer, wine, sparkling wine (champagne), wine cooler, wine spritzer, hard seltzer, sake, mead, kombucha, or cider. In some embodiments, the sugar source comprises wort, must, fruit juice, honey, rice starch, or a combination thereof. In some embodiments, the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit. In some embodiments, the sugar source is wort and the method further comprises producing the medium, wherein producing the medium comprises: contacting a plurality of grains with water; and boiling or steeping the water and grains to produce wort. In some embodiments, the method further comprises adding at least one hop variety to the wort to produce a hopped wort. In some embodiments, the method further comprises adding at least one hop variety to the medium. In some embodiments, the sugar source is must and the method further comprises producing the medium, wherein producing the medium comprises crushing a plurality of fruit to produce the must. In some embodiments, the method further comprises removing solid fruit material from the must to produce a fruit juice. In some embodiments, the method further comprises at least one additional fermentation process. In some embodiments, the method further comprises carbonating the fermented product.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows exemplary smoke taint remediation pathways and the chemical structures of smoke taint associated molecules described herein.

FIG. 2 shows the conversion ratio of five O-methyltransferases (OMTs) against five smoke taint phenols in vitro. The conversion ratio is defined as the gas chromatography-mass spectrometry (GC-MS) peak area of the methyl ether product (methylated volatile phenol) divided by the peak area of the phenolic substrate. Two clonal replicates of each enzyme were tested, resulting in two separate bars for each replicate (Clonal replicate 1 and Clonal replicate 2). Two technical replicate reactions were performed for each clone-substrate pair, resulting in error bars representing ±1 standard deviation. “No lysate” and “empty” vector controls were included, where no conversion was detected. A no substrate control was also performed for each enzyme and under these conditions neither substrate nor product was detectable by GC-MS. Phenol as a substrate was included in this experiment to inform structure-activity relationships, even though it is not classically recognized as a smoke taint species. The OMT enzymes correspond to Sigomt1, white campion; Siomt1, tomato; Rhoomt2, rose; Eomt1, basil; Ejomt1, loquat.

FIG. 3 shows conversion of non-volatile glucosides to volatile phenolic product by glucosidases. Acidified lysates from yeast expressing a heterologous glucosidase enzyme were incubated with non-volatile phenolic glucoside for 9 days. The plot shows the peak area of volatile phenolic product converted by the glucosidase from the non-volatile phenolic glucoside. Peak areas are normalized to the highest peak area for a given substrate across all experimental groups, with this maximum corresponding to 100% conversion.

FIGS. 4A-4B show images of representative of polyacrylamide gel electrophoresis of extracellular supernatant derived from the indicated yeast cell cultures expressing AoBgl1 fused to a secretion signal peptide.

FIG. 5 shows conversion of non-volatile phenolic glucosides to the indicated volatile phenols by AoBgl1 at fermentation product-relevant concentrations in vitro. Lysates from yeast cells expressing AoBgl1 were incubated with glucosides for 9 days in acidic buffer.

Controls with lysate omitted (Buffer, gray bars) indicate the extent of non-enzymatic conversion over the 9 day period.

FIG. 6 shows conversion of non-volatile phenolic glucosides to the indicated volatile phenols by purified β-glucosidase enzyme that was added to the mixture. β-glucosidase was incubated with glucosides for 9 days in acidic buffer. Controls without enzyme (Buffer, gray bars) indicate the extent of non-enzymatic conversion over the 9 day period.

FIG. 7 shows consumption of the indicated volatile phenol substrates by genetically engineered wine yeast strains expressing an OMT enzyme. The data show concentrations of the indicated volatile phenols after a 5 day wine fermentation in Pinot Noir grape juice with added phenol substrate. The parent strain controls (D254) indicate a decrease in concentration of phenols due to cellular sequestration or endogenous biotransformation alone. Strains y1375, expressing an OMT from basil, and y1376, expressing an OMT from loquat, indicate the change in phenol concentration due to OMT overexpression. Bars corresponding to strain y1375 and y1376 with 4-methylguaiacol are not visible due to full conversion of this phenol.

FIG. 8 shows production of the indicated volatile phenolic methyl ethers from phenols by genetically engineered wine yeast expressing an OMT enzyme. The data show concentrations of the indicated methylated phenols after a 5 day wine fermentation in Pinot Noir grape juice with spiked-in phenol substrate. The phenolic methyl ethers veratrole, 4-methylveratrole, 2-methylanisole, 3-methylanisole, and 4-methylanisole correspond to O-methylated products of guaiacol, 4-methylguaiacol, o-cresol, p-cresol, and m-cresol, respectively. Controls with the parent strain (D254) indicate a lack of endogenous production of phenol methyl ethers from phenols. Strains y1375, expressing an OMT from basil, and y1376, expressing an OMT from loquat, indicate phenolic methyl ether production due to OMT overexpression. Bars corresponding to y1375 and y1376 with 2-methylanisole and 4-methylanisole are not visible due to no detection of the predicted phenol methyl ether products. However, the corresponding phenols are consumed by strain y1376 (see, FIG. 7); the methyl ether products may be further metabolized by the strain.

FIG. 9 shows production of 4-methylveratrole from 4-methylguaiacol glucoside by a genetically engineered wine yeast strain expressing an O-methyltransferase enzyme in grape juice media containing β-glucosidase from almonds added to the mixture. The data show the concentrations of methylated phenol after a 5 day wine fermentation in Pinot Noir grape juice with added phenolic glucoside substrate. The juice control (“juice”) contains no 3-glucosidase and no yeast but does contain the glucoside substrate. 4-methylguaiacol glucoside is converted into 4-methylguaiacol by the β-glucosidase, which is then converted into 4-methylveratrole by the OMT enzyme. Controls with the parent strain (D254) and juice (bars not visible) indicate no endogenous production of the phenol methyl ether from phenol glucoside during fermentation. Strains y1375, expressing an OMT from basil, and y1376, expressing an OMT from loquat, exhibit phenolic methyl ether production due to the added β-glucosidase enzyme and overexpression of the OMT enzyme.

FIG. 10 shows production of 4-methylveratrole from 4-methylguaiacol glucoside by a genetically engineered wine yeast expressing an O-methyltransferase enzyme and a secreted β-glucosidase enzyme. The data show the concentration of 4-methylveratrole after a 5 day wine fermentation in Pinot Noir grape juice with added 4-methylguaiacol glucoside. The juice control (“juice”) contains no yeast but does contain the glucoside substrate. 4-methylguaiacol glucoside is converted into 4-methylguaiacol by the β-glucosidase, which is then converted into 4-methylveratrole by the OMT enzyme. Controls with the parent strain (D254) and juice (bars not visible) indicate no endogenous production of the phenolic methyl ether from phenol glucoside during fermentation. The y1386, y1387, y1388, and y1389 strains indicate phenol methyl ether production due to β-glucosidase and OMT overexpression. Strain y1386 corresponds to expression of an OMT from basil and A. oryzae β-glucosidase with N-terminal TFP5-1 secretion signal; strain y1387 corresponds to expression of an OMT from basil and A. oryzae β-glucosidase with N-terminal SED1 secretion signal; strain y1388 corresponds to expression of an OMT from loquat and A. oryzae β-glucosidase with N-terminal TFP5-1 secretion signal; and strain y1389 corresponds to expression of an OMT from loquat and A. oryzae β-glucosidase with N-terminal SED1 secretion signal.

### DETAILED DESCRIPTION

Significant efforts have been made within the wine community to understand the effect of smoke exposure on grapes and on the resulting wine produced from exposed grapes. In addition, methods have been developed to assess the risk of smoke taint in the final wine, and to mitigate the sensory effects of smoke taint on the final product. When wood burns, for example in wildfires, it releases lignin-derived volatile phenols that impart a smoky taste and aroma. Grapes exposed to smoke absorb these volatile phenols and metabolize them into non-volatile phenolic glycosides, which contain the phenol bound to a sugar molecule. See, e.g., Hayasaka, et al., J. Agric. Food Chem. (2010) 58: 10989-10998; Hayasaka, et al., J. Agric. Food Chem. (2013) 61: 25-33; Hayasaka, et al., J. Agric Food Chem. (2010) 58: 2076-2081; Szeto, et al., Molecules. (2020) 25. Chemical analysis of smoke affected grape juice and wine has identified the non-volatile phenolic glycosides to be mono- and di-glycosides, with a direct linkage between the phenol and beta-D-glucose moieties. The most common non-volatile phenolic glycosides include glucosides (containing glucose), gentiobiosides (containing two glucoses), and rutinosides (containing glucose and a terminal rhamnose). The risk of smoke taint depends on the duration of smoke exposure and the stage of development of the grapes, which are most susceptible between ripening and harvest. Once inside the grapes, smoke-derived compounds are mainly localized to the skins; consequently red wines, which are made in the presence of skins, tend to be the most significantly impacted by smoke taint.

At the start of the winemaking process, phenolic glycosides are extracted from the grape skins and/or grape juice into the fermenting wine. Hydrolysis of the glycosides during fermentation, aging, storage, and/or drinking releases volatile phenols and contributes to the sensory perception of smoke taint. See, e.g., Kennison, et al., J. Agric. Food Chem. (2008) 56: 7379-7383; Singh, et al., Australian Journal of Grape and Wine Research (2011) 17: S13-S21. The most potent of the volatile phenols are thought to be guaiacol and m-cresol, which have sensory detection thresholds of 23 μg/L and 20 μg/L in wine, respectively. See, e.g., Parker, et al., J. Agric. Food Chem. (2012) 60: 2629-2637. When present in wine at concentrations above these thresholds, off-flavors associated with smoke taint are perceptible. Additional volatile phenols, such as p-cresol, o-cresol, phenol, 4-methylguaiacol, also contribute to the perception of smoke taint, in part by providing an additive effect, even at concentrations below their sensory detection thresholds (approximately 62-65 μg/L). Volatile phenols with higher sensory detection thresholds, such as syringol and 4-methylsyringol, are thought to contribute less to perception of smoke taint but are still useful as markers for smoke exposure.

Concentrations of volatile phenols and phenolic glycosides in grapes and wines vary depending on the conditions during smoke exposure, but syringol gentiobioside is thought to be the most abundant smoke-taint compound, with concentrations as high as 1.5 mg/L. However, the potential for free syringol to influence the sensory perception of smoke, even if all of it were to be released from its bound form, is low, due to its high odor threshold. Guaiacol, 4-methylguaiacol, and cresol glycosides, while present concentrations in the tens to hundreds of micrograms per liter, do strongly affect the sensory perception of smoke as free phenols are released over time. For example, smoke-exposed grapes can release hundreds of micrograms per liter of free guaiacol from hydrolysis of guaiacol glycosides. As these concentrations are well above the threshold of detection, guaiacol glycoside is considered a major contributor to the overall profile of smoke-tainted wines.

More recently, smoke taint has become a problem for the beer brewing community.

Fires in the Pacific Northwest of the United States, where most U.S. hops are grown, have resulted in smoke-exposed hops which, like grapes, form phenolic glycosides from the volatile phenols the hops absorb. Research efforts have shown that the addition of smoke-exposed hops during the beer brewing process, regardless of preparation (whole cone, pellets, extracts) or timing of addition (kettle, whirlpool, during fermentation, can result in smoke-tainted beers. However, the smoke flavor is most intense when smoke-exposed hops are added during dry hopping—the practice of adding flavoring hops directly to beer in the fermentation tank, which has become increasingly popular in recent years.

Due to the detrimental effect of smoke exposure on wine and beer flavor, strategies to mitigate the impact of smoke taint are necessary. First, winemakers and brewers must decide whether or not to harvest smoke-affected grapes or hops. The Australian Wine Research Institute recommends assessing the risk for smoke taint by sending grape samples for analysis and conducting small-scale fermentations which can be analyzed by sensory analysis for smoke-related characteristics (“I can smell smoke—now what?” Grapegrower and Winemaker (2020) 672: 28-33). However, the decision to harvest a grape or hop crop may not be straightforward. Low or high concentrations of free and bound phenols may give a clear answer, intermediate levels may be difficult to interpret due to uncertainty regarding the different thresholds at which the fermented beverages (e.g., wines, beers) become smoke-tainted. In addition, small-scale fermentations take time and resources and may not be representative of the presence of volatile phenols in the final product after aging and storage.

During wine processing and fermentation, strategies used to mitigate smoke taint include excluding leaf material, keeping fruit cool, and minimizing the time fermentations contain grape skins. These strategies have limited effectiveness and are unlikely to reduce the concentration of polyphenols below the flavor detection threshold. Methods for remediation of finished, smoke-tainted wine include fining wine with activated carbon, treating with reverse osmosis, diluting wine with non-tainted wine, and adding tannins or oak chips to mask smoke sensory notes. Each of these strategies have significant challenges and limitations. Fining, for example, removes color, flavor, and desirable aroma compounds from the fermented beverages. Reverse osmosis also removes desirable aromas but also does not fully remove glycosides, resulting in the recurrence of smoke taint will return over time as the glycosides are hydrolyzed. Dilution of wine with non-tainted wine requires a high volume of non-tainted wine, and the addition of tannin or oak to the fermented beverage may produce a very different wine from the one intended.

The present disclosure describes the development of yeast strains that have been genetically engineered to hydrolyze non-volatile phenolic glycosides associated with smoke taint to produce one or more volatile phenols. Provided herein are modified yeast cells that have been engineered to express an enzyme having glycosidase activity or modified yeast cells that have been engineered to express an enzyme having glycosidase activity and an enzyme having O-methyltransferase (OMT) activity. Also provided herein are modified yeast cells that have been engineered to express an enzyme having O-methyltransferase (OMT) activity. Also provided herein are methods of producing a fermented beverage involving contacting the modified yeast cells with a medium comprising a sugar source comprising at least one fermentable sugar during a fermentation process. Also provided herein are methods of producing ethanol involving contacting the modified yeast cells with a medium comprising a sugar source comprising at least one fermentable sugar during a fermentation process. In some embodiments, the modified cells and methods of using such cells results in the production of compositions and fermented products from smoke-affected ingredients (e.g., grapes, hops) without the undesired presence of smote taint sensory notes. Glycosidase enzymes Aspects of the present disclosure relate to modified cells comprising a heterologous gene encoding an enzyme having glycosidase activity. Aspects of the present disclosure relate to modified cells comprising a heterologous gene encoding an enzyme having glycosidase activity and a heterologous gene encoding an enzyme having O-methyltransferase activity. The term “heterologous gene,” as used herein, refers to a sequence of nucleic acid (e.g., DNA) that contains the genetic instruction, which is introduced into and expressed by a host organism (e.g., a genetically modified cell) which does not naturally encode the introduced gene. The heterologous gene may encode an enzyme that is not typically expressed by the cell, a variant of an enzyme that the cell does not typically express (e.g., a mutated enzyme), an additional copy of a gene encoding an enzyme that is typically expressed in the cell, or a gene encoding an enzyme that is typically expressed by the cell but under different regulation.

Aspects of the present disclosure also relate to contacting a medium or a fermented product with an enzyme having glycosidase activity. In some embodiments, the enzyme is a recombinant enzyme, e.g., that is produced by an organism, isolated, and/or purified, and added to a medium or fermentation product. In some embodiments, the enzyme is extracted from a source, such as a microbial, yeast, mammalian, or plant source. Accordingly, any of the glycosidase enzymes described herein (e.g., AoBgl1) may be expressed as a heterologous enzyme in a genetically modified cell or provided in the form of a recombinant and/or purified enzyme. In some embodiments, the glycosidase is from sweet almond, E coli, Thermotoga neapolitana, Bacteroides fragilis, Clostridium thermocellum, Rhizobium etli, Streptococcus pyogenes. In one example, the glycosidase enzyme is purified from almonds, and provided in lyophilized form, for example from Millipore Sigma. In one example, the glycosidase enzyme is recombinant human glycosidase, and provided in lyophilized form, for example from Bio-Techne Corporation.

Glycosidases are enzymes that catalyze the hydrolysis of glycosidic bonds between two or more sugars, or between a sugar and another chemical residue (e.g., a phenol and a sugar). In some embodiments, the glycosidase removes a terminal sugar residue from a saccharide or glycoside. Glycosidases tend to have distinct substrate specificities and catalytic activities and are typically categorized based on the type of sugar residue that is hydrolyzed. In some embodiments, the sugar is glucose or rhamnose. In some embodiments, the glycosidase is a glucosidase. In some embodiments, the glycosidase is a β-glucosidase, such as a β-d-glucopyrranoside glucohydrolase. In some embodiments, the glycosidase is a rhamnosidase. In some embodiments, the glycosidase is an alpha-L-rhamnosidase.

Hydrolysis of non-volatile phenolic glycosides results in release of free, volatile phenols that may impart a smoky off-flavor to fermented product before, during, or after fermentation. Phenols, in the form of non-volatile phenolic glycosides, may be absorbed into a fermentation substrate (e.g., grapes, hops), for example, when the fermentation substrate is exposed to environmental smoke. Over time, the phenols are released from the glycoside to become free, volatile phenols that impart an undesired smoky off-flavor.

The modified cells described herein are genetically modified to express a heterologous gene encoding an enzyme having glycosidase activity which can hydrolyze the non-volatile phenolic glycosides associated with smoke taint thereby producing free, volatile phenols. In some embodiments, the enzyme having glycosidase activity is a recombinant and/or purified enzyme which can hydrolyze the non-volatile phenolic glycosides associated with smoke taint thereby producing free, volatile phenols. Volatile phenols can then be removed by methods, such as filtration (e.g., reverse osmosis), contacting with a fining agent, or by further chemical modification (e.g., through conversion to a modified form (e.g., methylation)).

In some embodiments, the glycosidase catalyzes removal (release) of a sugar moiety from one or more non-volatile glycoside associated with smoke taint. In some embodiments, the glycosidase catalyzes removal (release) of a glucose moiety from one or more non-volatile glycoside associated with smoke taint. In some embodiments, the glycosidase catalyzes removal (release) of a rhamnose from one or more non-volatile phenolic glycoside associated with smoke taint.

As used herein, the term “non-volatile phenolic glycoside associated with smoke taint” refers to a sugar moiety bound to a phenol that is in a form that does not evaporate into a gas form under particular conditions. Examples of non-volatile phenolic glycoside associated with smoke taint include, without limitation, glucosides, gentiobiosides, and rutinosides. In some embodiments, the glycosidase catalyzes removal (release) of a glucose moiety from a glucoside associated with smoke taint. In some embodiments, the glycosidase catalyzes removal (release) of at least one glucose moiety from a gentiobioside associated with smoke taint. In some embodiments, the glycosidase catalyzes removal (release) of a glucose moiety and/or a rhamnose from a rutinoside associated with smoke taint.

As described herein, hydrolysis of a non-volatile phenolic glycoside results in the production of one or more volatile phenols. Examples of volatile phenols include, without limitation, guaiacol, m-cresol, p-cresol, o-cresol, phenol, 4-methylguaiacol, syringol, and/or 4-methylsyringol. In some embodiments, the hydrolysis of a non-volatile phenolic glycoside results in the production of any one or more of guaiacol, m-cresol, p-cresol, o-cresol, phenol, 4-methylguaiacol, syringol, and/or 4-methylsyringol.

In some embodiments, the heterologous gene encoding an enzyme with glycosidase activity is a wild-type (naturally occurring) glycosidase gene (e.g., a gene isolated from an organism). In some embodiments, the enzyme having glycosidase activity is obtained from a bacterium. In some embodiments, the enzyme having glycosidase activity is obtained from a plant. In some embodiments, the enzyme having glycosidase activity is obtained from a yeast or fungus.

In some embodiments, the enzyme having glycosidase activity is derived from Aspergillus aculatus, Aspergillus aculatus, Aspergillus terreus, Aspergillus nidulans, Aspergillus niger, Aspergillus oryzae, Lactobacillus plantarum, Lactobacillus acidophilus, Thermomicrobia PRI-1686, Candida molischiana, Bacillus polymyxa, Olea europaea, Saccharomyces cerevisiae, Thermoascus aurantiacus, Aspergillus niger, Trichoderma reesei, Neurospora crassa, Oenococcus oeni, Prunus dulcis, Bacillus circulans, or Sinorhizobium meliloti.

In some embodiments, the enzyme having glycosidase activity is RhaB derived from Aspergillus aculatus, RhaA derived from Aspergillus aculatus, AtRha derived from Aspergillus terreus, AndRha derived from Aspergillus nidulans, AngRha derived from Aspergillus niger, AorhaA derived from Aspergillus oryzae, RhaB1 derived from Lactobacillus plantarum NCC245, RhaB2 derived from Lactobacillus plantarum NCC245, LpRam1 derived from Lactobacillus acidophilus, RhmA derived from Thermomicrobia PRI-1686, RhmB derived from Thermomicrobia PRI-1686, bgln derived from Candida molischiana, bglA derived from Bacillus polymyxa, OepGLU derived from Olea europaea, EGH1 derived from Saccharomyces cerevisiae, Tabgl1 derived from Thermoascus aurantiacus IF09748, AoBGL1 derived from Aspergillus oryzae, AnBGL1 derived from Aspergillus niger, TrBGL2 derived from Trichoderma reesei, TrBGL1 derived from Trichoderma reesei, BGL1 derived from Neurospora crassa, BGL2 derived from Neurospora crassa, bglH derived from Aspergillus oryzae, OoBgl derived from Oenococcus oeni, Ph691 from Prunus dulcis, Ph692 from Prunus dulcis, BsBglA from Bacillus circulans, or SmBgl from Sinorhizobium meliloti.

In some embodiments, the glycosidase is a rhamnosidase. In some embodiments, the glycosidase is a alpha-L-rhamnosidase (also referred to as alpha-L-rhamnoside rhamnohydrolase). An exemplary enzyme having glycosidase (alpha-L-rhamnosidase) activity is RhaB from Aspergillus aculeatus. The Aspergillus aculeatus RhaB is provided by the amino acid sequence set forth by SEQ ID NO: 1, which corresponds to UniProtKB Accession No. AF284762.

(SEQ ID NO: 1)
MHIITPLLIPAVLVAAARVPYREYILAPSSRVIVPASVRQVNGSVTNAAGLTGSSLGTAV
FHGVSSVTYDFGKNVAGIVSLTVGSSSSPSAFLGVTFSESSLWASSEACDATGNSGLDAP
LWFPVGQKAGTYTPDSKYVRGGFRYLTVVSNTSATIPLNSLHITFTAAPDQDLQAYQGWF
HSNDELLNEIWYAGAYTNQLCTIDPTYGSASSETISTSGLNYWYNNLTIANGTSTVTDGA
KRDRAVWPGDMSISLESIAVSINDLYSVRMGLEALLALQSSEGQLPWGGKPFNIDVSYTY
HLHSLIGMSFLYRFSGDKVWLSNYWGQYSKGVEWAVRSVADGVKSAANQLLWDDQAGLYR
DNQTTELHPQDGNAWAVKSNLTLSGSQNRAISQALKARWGRYGAPAPEAGATISPFIGGF
EIQSHFLANQPDVALDMIRLQWGFMLRDPRMTQSTLIEGYSTDGSIHYAPYANDARISHA
HGWSTGPTYALTAYAAGLQLLGPAGNSWLIAPQPGGLTSIDCGFATALGVFSVVFERDSV
GRYNSFSFGAPTGTTGRIELPGVRGTLVSTTGQRVQLVNGTASGLRGGKWKLIESAD

An exemplary enzyme having glycosidase (alpha-L-rhamnosidase) activity is RhaA from Aspergillus aculeatus. The Aspergillus aculeatus RhaA is provided by the amino acid sequence set forth by SEQ ID NO: 2, which corresponds to UniProtKB Accession No. AF284761.

(SEQ ID NO: 2)
MLWSSWILTPALLAIGSHAVPFEDYILAPQSRTLNPSLVYQVNGTVDNPEALVGLTTHQT
TLHGKSSVTYDFGRNIAGIVSLDVTSVSSKAAQLGVTFTESSLWISSEACDATSDAGLDS
PLWFSVGHGPGTYGADKKHLRGAFRYLTLVNNSTATISLDGLSINYTAAPTQDLRGYKGY
FHSSDELINRIWYAGAYTLQLCTIDPTTGDSLIWLGVISSSDNITLPQTDSWWNNYTITN
GSTTLVDGAKRDRLVWPGDMSIAIESAAVSTADLESVRTALESLFVLQKANGQLPYAGRP
FLDIVSFTYHLHSLIGASSYYQYTGDRSWITRYWGQYKKGLQWALSSLDNSGLANITASA
DWLRFGMGGHNIEANAILYYVLNDAISLASSLDDRANVGNWSTAASKIKAAANARLWDAQ
NSLYRDNETTTLHPQDGNAWAIKANLTLSSNQSEAISSALAARWGPYGAPAPEAGSTVSP
FIGGFELQAHYLANEPDRALDLLRLQWGFMLDDPRMINSTFIEGYSTDGSLAYAPYRNTP
RVSHAHGWSTGPTSALTHYTAGLRLTGPAGSTWLFKPQPGNLTEVQAGFETQLGLFATQY
QKSATGTFQQLTFTAPNGTSGSVEIEGATGQLISKRGQAVKLVNGKARGLQGGTWTLKGL

An exemplary enzyme having glycosidase (alpha-L-rhamnosidase) activity is AtRha from Aspergillus terreus. The Aspergillus terreus AtRha is provided by the amino acid sequence set forth by SEQ ID NO: 3, which corresponds to UniProtKB Accession No. AFH54529.

(SEQ ID NO: 3)
MALSISQVAFEHHRTALGIGETQPRVSWRFDGNVSDWEQRAYEIEVKRAGHDADVERSES
SDSVLVPWPSSPLQSGEEATVRVRSFGSDGQHDTPWSDAVTVEPGLLTPDDWHDAVVIAS
DRPTEVDATHRPIQFRKEFSVDDSYVSARLYITALGLYEARINDQRVGDHVMAPGWQSYQ
YRHEYNTYDVTDLLKQGPNAIGVTVGEGWYSGRIGYDGGKRNIYGDTLGLLSLLVVTKSD
GSKLYIPSDSSWKSSTGPIISSEIYDGEEYDSRLEQKGWSQVGFNSTGWLGTHELSFPKE
RLASPDGPPVRRVAEHKLANVESSASGKTVLDFGQNLVGWLRIRVKGPKGQTIRFVHTEV
MENGEVATRPLRQAKATDHFTLSGEGVQEWEPSFTYHGFRYVQVDGWPADTPLDENSVTA
IVVHSDMERTGYFECSNPLISKLHENILWSMRGNFFSIPTDCPQRDERLGWTGDIHAFSR
TANFIYDTAGFLRAWLKDARSEQLNHSYSLPYVIPNIHGNGETPTSIWGDAIVGVPWQLY
ESFGDKVMLEEQYGGAKDWVDKGIVRNDVGLWDRSTFQWADWLDPKAPADDPGDATTNKY
LVSDAYLLHSTDMLANISTSLSKGEEASNYTEWHAKLTKEFQKAWITSNGTMANETQTGL
ALPLYFDLFPSAEQAQSAAKRLVNIIKQNDYKVGTGFAGTHLLGHTLSKYGESDAFYSML
RQTEVPSWLYQVVMNGTTTWERWDSMLPNGSINPGQMTSFNHYAVGSVGSWLHEVIGGLS
PAEPGWRRINIEVVPGGDLQQASTKFLTPYGMASTKWWLDGQDQSCGGFDFHLVAEVPPN
TRATVVLPGKGGEKVDVGSGVHEYHVRCVKL

An exemplary enzyme having glycosidase (alpha-L-rhamnosidase) activity is AndRha from Aspergillus nidulans. The Aspergillus nidulans AndRha is provided by the amino acid sequence set forth by SEQ ID NO: 4, which corresponds to UniProtKB Accession No. CBF86381.1.

(SEQ ID NO: 4)
MISLPLLALATGAVASASCWRNTSCTGPSKPSFPGPWDSNNFAPGSRLIQPKSILSLPDG
TYISDYTSDSHPSITTEDAGLVFDFGLEVGGIVTIDYSSSVPNTTLGLAFTEAKDYIGRT
SDNSNGGTGADGALATLLTEGEGSYTMPDSQLRGGFRYLALFIPSSSSSNASLTIQSITL
ELAFQPTWSNLRAYQGYFHSSDPVLNKAWYAGAYTLQTNSVPRTTCRVSVSSATGWNNNA
VCGPGETLLLDGAKRDRWVWIGDMGVAVPSASVSTGDTESTKNALLAIWDNQTPSGLLPK
AGPPYLRADSDTYHLWTIIGTYNYFLFTEDYDFLAGIWQKYVKALDYSLAKITPLGIMNA
TQTADWGRWNYGTLASSANMLLYRSLTTAAFLAPYAGDNPENYTDLASTLRSAIVTHLYD
SAVGAFRDSPNSTLYPQDANSMALAFSFFSQSPLNCSSSENLAEAARVSSYLESNWTPIG
PEVPELPNNISPFISSIELEGHFASGHADRAIELIRMLWGWYLAHPNGTQSTVPEGYLVD
GSWGYRGDRGYRNDPRYVSHAHGWSSGPTSTLTEYAVGLKITKPKGSEWSLRPASFGIQG
FEEAQAGFTTGLGKFKAAFRVQGKRATVTWDTPAGTKGWVQLPGEEGSWVEGGMGSLTVK
L

An exemplary enzyme having glycosidase (alpha-L-rhamnosidase) activity is AndRha from Aspergillus nidulans. The Aspergillus nidulans AndRha is provided by the amino acid sequence set forth by SEQ ID NO: 5, which corresponds to UniProtKB Accession No. FR873475.

(SEQ ID NO: 5)
MSLSISGVTFEHHRSALGIGEPSPRISWRFDGTVSNWTQSAYEIEINRAGQANTERVNSS
DSVLVPWPSDPLQSGEEATVRVRSFGRANQPDAPWSDPVTVEPGLLDEDDWQSAVAIVSD
RETEVNATHRPIYFRKDFDVDEEILSARLYITALGVYEAEINGQPVGDHVLAPGWQAYSH
RHEYNTYDVTDLLQTGDNTIGVTVGEGWYAGALTWSMTRNIYGDTLGLLSLLSIATADGK
TIYVPSDETWQSSTGPIIASEIYNGETYDSTQAIEGWSQPGFDASGWLGTHEVTFDKSVL
AAPDAPAVRRVEERRLESVEKSASGKTVLDFGQNLVGWLRVRVKGPRGSTISFVHTEVME
NGEVATRPLRNAKATDNLTLSGEEQEWEPSFTFHGFRYVQVTGWPEETELNADSVTAIVI
NSDMEQTGFFSCSNPLLNKLHENIIWSMRGNFLSIPTDCPQRDERLGWTGDIHAFARTAN
FIYDTSGFLRGWLRDAYSEQLENNYAPPYVIPNVLGPGSPTSIWGDAIVSVPWDLFQTYG
DKAMLSEQYAGATAWLDKGILRNEAGLWNRSTFQYADWLDPLAPPDDPGAATTNKYLVSD
AYLIHSTELVANISAYLDRPDDAERYAADRADLTRAFQKAWISANGTVANETQTGLTLPL
YFKLFERPEHYTDAVSRLVDIIKENEYKVGTGFAGTHLLGHTLSAYNASSTFYNTLLQED
VPGWLFQVLMNGTTTWERWDSMLANGSVNPGEMTSFNHYAVGSVGAWMHENIGGLRPIEP
GWRRFAVDVKVGGGLSSAQERFLSPYGSAESSWEVRDGKFMLGVKVPPNSEAVVSLPGAP
TRGKKEVIVGSGMHRFESTLG

An exemplary enzyme having glycosidase (alpha-L-rhamnosidase) activity is AngRha from Aspergillus niger. The Aspergillus niger AngRha is provided by the amino acid sequence set forth by SEQ ID NO: 6, which corresponds to UniProtKB Accession No. XP_001389086.1.

(SEQ ID NO: 6)
MWSSWLLSALLATEALAVPYEEYILAPSSRDLAPASVRQVNGSVTNAAALTGAGGQATEN
GVSSVTYDFGINVAGIVSVDVASASSDSAFIGVTFTESSMWISSEACDATQDAGLDTPLW
FAVGQGAGLYTVEKKYNRGAFRYMTVVSNTTATVSLNSVKINYTASPTQDLRAYTGYFHS
NDELLNRIWYAGAYTLQLCSIDPTTGDALVGLGVITSSETISLPQTDKWWINYTIINGSS
TLTDGAKRDRLVWPGDMSIALESVAVSTEDLYSVRTALESLYALQKPDGRLPYAGKPFFD
TVSFTYHLHSLVGAASYYQYTGDRAWLTRYWGQYKKGVQWALSSVDSTGLANITASADWL
RFGMGAHNIEANAILYYVLNDAISLAQTLNDNAPIRNWTTTAARIKTVANELLWDDKNGL
YTDNETTTLHPQDGNSWAVKANLTLSANQSAIVSESLAARWGPYGAPAPEAGATVSPFIG
GFELQAHYQAGQPDRALDLLRLQWGFMLDDPRMINSTFIEGYSTDGSLAYAPYTNTPRVS
HAHGWATGPTSALTIYTAGLRVTGPAGATWLYKPQPGNLTQVEAGFSTRLGSFASSFSRS
GGRYQELSFSTPNGTTGSVELGDVSGQLVSDRGVKVQLVGGKASGLQGGKWKLSNN

An exemplary enzyme having glycosidase (alpha-L-rhamnosidase) activity is AorhaA from Aspergillus oryzae. The Aspergillus oryzae AorhaA is provided by the amino acid sequence set forth by SEQ ID NO: 7, which corresponds to UniProtKB Accession No. BAE58354.1.

(SEQ ID NO: 7)
MRLPWSAWLLSPALLAVACYGVPYNEYILAPASRHLVPFEVLEVNGSVTDPSSLTQSTGG
NATFNGPASVTFDFGRNIAGIVSLDIGSSSTRDAFIGVIFTESSLWISSQACDATADSGL
DSPLWFPVGRGAGTYTADKKHNRGAFRYMTLVTNTTAVVSVRNVQINYTAAPSQDLRAYT
GYFHSNDELLNRVWYAGAYTNQICTIDPSTGDALPFLGVISSDSNITLPETNPWYSNYTI
SNGSSTLTDGAKRDRLIWPGDMSIALESVSVSTADLYSVRTALETLLSQQRSDGRLPYAS
EPFLDLVSYTYHLHSLIGVSYYYRHSGDRAWLSKYWGQYQKGLQWALSSVDNTGLANITA
SSDWLRFGMGGHNIEANAILYFVLNEAQELSQAINNHTNANWTKIASGIKSATNKNLWDA
NNGLYKDNETTTLYPQDGNAWAIKANLTLSTNQSSTISSALSSRWGNYGAPAPEAADAVS
PFIGGFEIQAHFLANQPQKALDLIRLQWGFMLDDPRMINSTFIEGYSTDGTLHYAPYTND
ARVSHAHGWSTGPTAALSFFVAGLHLTGSAGATWRFAPQPGDLTSVDAGYTTALGLFSTT
FKRSENGDYQELTFTTPQGTTGDVDLAGAEGTLVSADGERVFLVKGTATGITGGSWNLEV
ASQ

An exemplary enzyme having glycosidase (alpha-L-rhamnosidase) activity is RhaB1 from Lactobacillus plantarum NCC245. The Lactobacillus plantarum NCC245 RhaB 1 is provided by the amino acid sequence set forth by SEQ ID NO: 8, which corresponds to UniProtKB Accession No. FJ943501.

(SEQ ID NO: 8)
MSKEAVWLWYPGDFEIHQGMLQNFKREERGMGWPAYWYIDDCNRNVNFKRHYDLKESTQF
TVLAKGTGYVDVNGTKYRLNHAINCDAGATDIQVFVGNVQGLPTIYIVGEIIKSDSGWLA
SNFVTTLPAGHDILYTDRNQDPNVIEYRTEKVVAKAQQAVDGGVLYDFGRAVNGTVTVKT
NGPVTLCYGESETEARDVEMCYYKQSDVTATTKVRKRAFRYVFVPHCQLGDIELTAMHEY
IPKNNPSSFTSDNKLINQIWNVATETLNLCSDLFFIDGIKRDRWIWAGDAYQANFINQYS
FFNEDIDKRTLLALRGQDDIKQHMNTIVDYSMLWVIGVLNHYQMTGDREFLKIIYPKLES
MVQYFIQQTNEHGFIYGRKNDWIFVDWSEMDKQGTVAAEQILLLEDYKTIMTCGEVLGKD
VAGYQAKYDQLFKNLMKYFWDDEQGAFIDSYESGKRHVTRHANIFAILEDVVDENKQQLI
LKNVLLNNAITQITTPYFKFFEQDALCKLGEQHRVYQVLLDYWGGMLDRGAVTFWEEFDP
SQHGKDMYAMYGDPYGKSLCHAWGASPIYLLGRHFVGLRPTAPGYQTFEIKPELSEFHHL
HTVLPIKGGTVTVVKDQHQLSVTASRAGGTLIVDGQRQSLEPNRTAVVPV

An exemplary enzyme having glycosidase (alpha-L-rhamnosidase) activity is RhaB2 from Lactobacillus plantarum NCC245. The Lactobacillus plantarum NCC245 RhaB2 is provided by the amino acid sequence set forth by SEQ ID NO: 9, which corresponds to UniProtKB Accession No. FJ943501.

(SEQ ID NO: 9)
MAFTFQINNDVQFRHNQALLDKSASYRPILKETQVKAASIVALELDTQYLEGWGVKQIAP
IERLSSYELKRDDQIIIDFGDHQVGQFSININAVGSPMDAPLCFKIKFAEMPAELARKSE
DYDGWLSKSWIQEETVHLDVLPTTLTLPRRYSFRYAEITVVDTSPKWRAVFSNPVVTATS
AVDTATVHQPELADAQLQRIYEVGLKTLADCMQDVFEDGPKRDRRLWIGDLRLQALANYA
TFKDTDLVKRCLYLFGAMPTTAGRIPANVFTKPTAVPDDTELFDYSLFFISILADYEAFS
SDKTVLNDLYRVAKNQMDLALAQVTSEGKLKLTEENPVFIDWSNDEDKETAGQAIIIYTL
KQFITLAELVNDTSLETYTAILRKLNQYAKTQLFDSQSGLFVSGDQREVNVASQVWMTLA
HVLDPEQTTALMQTTVTKLFPITGIATPYMYHHITEALFEAGLKQEAVQLMKDYWGKMIT
LGADTYWEAFDPNQPDYSPYGSPILNSYCHAWSCTPVYLINKYLV

An exemplary enzyme having glycosidase (alpha-L-rhamnosidase) activity is RhmA from Thermomicrobia PRI-1686. The Thermomicrobia PRI-1686 RhmA is provided by the amino acid sequence set forth by SEQ ID NO: 10, which corresponds to UniProtKB Accession No. AAR96046.

(SEQ ID NO: 10)
MLRIDRVKVERSRDGLGLGTGRPRLCWRVETDIRDWRQAAYEVELYDGSGQLVGSTGRVE
SGESVWVAWPFEALGSRQRAGVRVRVWGEDGSESDWSDLQWLEVGLLARDDWQGAFITPD
WEEDTSVANPCPYLRKTFSLPGGVRRARLYVTGLGVYEVELNGQRVGDHVLSPGWTSYRH
RLLYETFDVTGLLREGDNCLGAILGDGWYRGRLGFGGGRRNLYGERLALLAQLEVELEDG
SRQVVVTDGSWRAHRGPILESGIYDGEVYDARLEMPGWSTPEYDDSEWAGTRELGWPTES
LEPLEVPARRTQEVAPREILRSFSGKTIVDFGQNLVGRVRLRVSGPRGQRVRLRHAEVLE
GGELCTRTLRTARATDEYVLRGDGEEEWEPRFTFHGFRYVEVEGWPGELRAEDLVAVVCH
SDMERIGWFGCSDPLVERLHENVVWSMRGNFLHIPTDCPQRDERLGWTGDIQVFSPAACF
IYDASGELTSWLRDVALDQDESGAVPFVVPNALGGQVIPAAAWGDAAVIVPWVLYQRYGD
AGVLEAQWPSMRAWVDCIKTIAGPARLWNKGFQFGDWLDPAAPPDNPAAARTDPYIVASA
YFARSAEIVGLSAQVLGMQDMAEEYLGLASEVREAFNREYVTPNGRVVSDAQTAYSLAIG
FALLPTQEQRQHAGERLAELVRAEGYKIGTGFVGTPLICDALCATGHHDVAYRLLMSREC
PSWLYPVIMGATTIWERWDSLRPDGSVNPGEMTSFNHYALGAVADWLHRVVGGLAPAEPG
YRKLRIQPVPGGGLSYARARHVTPYGTAECSWRTEGGEIEVRVVVPPNTSAQVVLPGSGR
EVEVGSGEHVWRYAFEAHRYPPVTLDTPLKEILEDAEAWEVLTRHFPEVASMPPRRLERI
GTIRDLAASVVAFNERVGRLERELQALSRERS

An exemplary enzyme having glycosidase (alpha-L-rhamnosidase) activity is RhmB from Thermomicrobia PRI-1686. The Thermomicrobia PRI-1686 RhmB is provided by the amino acid sequence set forth by SEQ ID NO: 11, which corresponds to UniProtKB Accession No. AAR96047.

(SEQ ID NO: 11)
MQWQASWIWLEGEPSPRNDWVCFRKSFELDRSASPLEEAKLSITADSRYVLYVNGQLVGR
GPVRSWPFEQSYDTYDLRHLLHPGRNCLAVLVTHFGVSTFSYVRGRGGLLAQLELSSGDD
RTTIGTDGSWKVHRHLGYSRRTTRISPQQGFVEQLDARAWSSEWKDLMYDDSGWEDAMIV
GPVGTPPWEQLVPRDIPFLTEEVLHPTRVVSLHSTVPPKIAVAVDMRAIMMPDSADHAEQ
VQYAGFLATILRTDGEGTARLLLSKPWVGDGIAASINGQVYGAELMSRTPTGRELEVELS
AGDNLLLVYVCGSDHADPLRLALDSDLGLELVSPTGGESAFVAIGPLASRVVRNEDFSQP
LEYDETAVRRISSCASVADLRAWSHLPRSVPPELVSPADVFTLCTWPRQRTELTTGKELE
AMVFPSKDPGLVPILRAGDTELVLDFGQEVSGYLFLDVEASEGTLIDLYGFEFMEDDYRQ
DTVGLDNTLRYTCREGRQHYVSPQRRGLRYLMLTVREARAPLRVHGVGVVQSTYPVSQVG
TFRCSDPLLNDIWEISRLTTKLCMEDTFVDCPAYEQTFWVGDSRNEALTAYYLFGAEELV
RRCLRLVPGSRRYTPLYMDQVPSGWVSVIPNWTFLWVMACREYYERTGDLAFVQDIWPDI
QYTLDHYLQHINDDGLLEISAWNLLDWAPIDQPNSGVVTHQNCFFVRALKDADELGQSAG
DETAGRYAERARELAAAINTHLWSDEHKAYIDSIHADSTRSSVISMQTQVVALLTGVAEG
DRAEVVRSHIASPPAGWVQIGSPFMSFFLYEAMVRQGMYAQMLEDIRQKYGLMLEHGATT
CWETFPGALGARYTRSHCHAWSAAPGYFLGAYVLGVRPGGPGWHRVIVAPQPCDLAWARG
SVPLPRGDRVDVSWRREGQKLLLRVERPQEVELEVVPPEEYELELDERVRQTTQ

An exemplary enzyme having glycosidase (beta-D-glucosidase) activity is Bgln from Candida molischiana. The Candida molischiana Bgln is provided by the amino acid sequence set forth by SEQ ID NO: 12, which corresponds to UniProtKB Accession No. U16259.1.

(SEQ ID NO: 12)
MKSTIIILSVLAAATAKNISKAEMENLEHWWSYGRSDPVYPSPEISGLGDWQFAYQRARE
IVALMTNEEKTNLTFGSSGDTGCSGMISDVPDVDFPGLCLQDAGNGVRGTDMVNAYASGL
HVGASWNRQLAYDRAVYMGAEFRHKGVNVLLGPVVGPIGRVATGGRNWEGFINDPYLAGA
LVYETTKGIQENVIACTKHFIGNEQETNRNPSGTYNQSVSANIDDKTMHELYLWPFQDSV
RAGLGSIMGSYNRVNNSYACKNSKVLNGLLKSELGFQGFVVSDWGGQHTGIASANAGLDM
AMPSSTYWEEGLIEAVKNGTVDQSRLDDMATRIIAAWYKYARLDDPGFGMPVSLAEDHEL
VDARDPAAASTIFQGAVEGHVLVKNENALPLKKPKYISLFGYDGVSTDVNTVGGGFSFFS
FDVKAIENKTLISGGGSGTNTPSYVDAPFNAFVAKAREDNTELSWDFTSAEPVANPASDA
CIDFINAAASEGYDRPNLADKYSDKLVEAVASQCSNTIVVIHNAGIRLVDNWIEHENVTG
VILAHLPGQDTGTSLIEVLYGNQSPSGRLPYTVAKKASDYGGLLWPTEPEGDLDLYFPQS
NFTEGVYIDYKYFIQKNITPRYEFGYGLTYTTEDYSELEVDAITNQSYLPPDCTIEEGGA
KSLWDIVATVKFTVTNTGDVAAAEVPQLYVGIPNGPPKVLRGFDKKLIHPGQSEEFVFEL
TRRDLSTWDVVAQNWGLQAGTYQFYVGRSVFDVPLTSALVETN

An exemplary enzyme having glycosidase (beta-glucosidase) activity is OepGLU from Olea europaea (olive). The Olea europaea OepGLU is provided by the amino acid sequence set forth by SEQ ID NO: 13, which corresponds to UniProtKB Accession No. KX278417.

(SEQ ID NO: 13)
MDIQSNVLTITSGSSPTDTSSNGQAAKSTKERIKRSDFPSDFVFGAATASYQVEGAWNEG
GKGMSNWDYFTQSQPGGISDFSNGTIAIDHFNMFKDDVVVMKKLGLKAYRFSLSWPRILP
GGRLCHGVSKEGVQFYNDLIDALLAADIEPYITIFHWDIPQCLQLEYGGFLHERVVQDFI
EYSEICFWEFGDRVKYWITLNEPWSFTVQGYVAGAFPPNRGVTPKDTEETKKHARLHRGG
GKLLTAFKYGNPGTEPYKVAHNLILCHAHAVDIYRTKYQESQGGKIGITNCISWNEPLTD
SQEDKDAATRGNDFMLGWFVEPVVTGEYPESMIKNVGDRLPKFSEKEEKLVKGSYDELGI
NYYTSTYTSDDPTKPTTDSYLTDSRIKTSHERNKVPIGAQAGSDWLYIVPWGIYRVMVDM
KKRYNDPVIYITENGVDEVNDKSKTSTEALKDDIRIHYHQEHLYYLKLAMDQGVNLKGYF
IWSLFDNFEWAAGFSVRFGVMYVDYANGRYTRLPKRSAVWWRNFLTKPTAVPLKNEPEKS
EDRRKRLRGST

An exemplary enzyme having glycosidase (beta-glucosidase) activity is EGH1 from Saccharomyces cerevisiae. The Saccharomyces cerevisiae EGH1 is provided by the amino acid sequence set forth by SEQ ID NO: 14, which corresponds to UniProtKB Accession No. NP_012272.3.

(SEQ ID NO: 14)
MPAKIHISADGQFCDKDGNEIQLRGVNLDPSVKIPAKPFLSTHAPIENDTFFEDADKVSF
INHPLVLDDIEQHIIRLKSLGYNTIRLPFTWESLEHAGPGQYDFDYMDYIVEVLTRINSV
QQGMYIYLDPHQDVWSRFSGGSGAPLWTLYCAGFQPANFLATDAAILHNYYIDPKTGREV
GKDEESYPKMVWPTNYFKLACQTMFTLFFGGKQYAPKCTINGENIQDYLQGRENDAIMTL
CARIKEKAPELFESNCIIGLESMNEPNCGYIGETNLDVIPKERNLKLGKTPTAFQSFMLG
EGIECTIDQYKRTFFGFSKGKPCTINPKGKKAWLSAEERDAIDAKYNWERNPEWKPDTCI
WKLHGVWEIQNGKRPVLLKPNYFSQPDATVFINNHFVDYYTGIYNKFREFDQELFIIIQP
PVMKPPPNLQNSKILDNRTICACHFYDGMTLMYKTWNKRIGIDTYGLVNKKYSNPAFAVV
LGENNIRKCIRKQLSEMQKDAKSMLGKKVPVFFTEIGIPFDMDDKKAYITNDYSSQTAAL
DALGFALEGSNLSYTLWCYCSINSHIWGDNWNNEDFSIWSPDDKPLYHDTRAKTPTPEPS
PASTVASVSTSTSKSGSSQPPSFIKPDNHLDLDSPSCTLKSDLSGFRALDAIMRPFPIQI
HGRFEFAEFNLCNKSYLLKLVGKITPEQITVPTYIFIPRHHFTPSRLSIRSSSGHYTYNT
DYQVLEWFHEPGHQFIEICAKSKSRPNTPGSDTSNDLPAECVIS

An exemplary enzyme having glycosidase (beta-glucosidase) activity is Tabgl1 from Thermoascus aurantiacus IFO9748. The Thermoascus aurantiacus IFO9748 Tabgl1 is provided by the amino acid sequence set forth by SEQ ID NO: 15, which corresponds to UniProtKB Accession No. DQ114397.

(SEQ ID NO: 15)
MRLGWLELAVAAAATVASAKDDLAYSPPFYPSPWMDGNGEWAEAYRRAVDEVSQLTLAEK
VNLTTGVGWMQEKCVGETGSIPRLGFRGLCLQDSPLGVRFADYVSAFPAGVNVAATWDKN
LAYLRGKAMGEEHRGKGVDVQLGPVAGPLGRHPDGGRNWEGESPDPVLTGVLMAETIKGI
QDAGVIACAKHFIGNEMEHFRQASEAVGYGFDITESVSSNIDDKTLHELYLWPFADAVRA
GVGSFMCSYNQVNNSYSCSNSYLLNKLLKSELDFQGFVMSDWGAHHSGVGAALAGLDMSM
PGDTAFGTGKSFWGTNLTIAVLNGTVPEWRVDDMAVRIMAAFYKVGRDRYQVPVNEDSWT
KDEYGYEHALVGQNYVKVNDKVDVRADHADIIRQIGSASVVLLKNDGGLPLTGYEKFTGV
FGEDAGSNRWGADGCSDRGCDNGTLAMGWGSGTADFPYLVTPEQAIQNEILSKGKGLVSA
VTDNGALDQMEQVASQASVSIVFVNADSGEGYINVDGNEGDRKNLTLWKGGEEVIKTVAA
NCNNTIVVMHTVGPVLIDEWYDNPNVTAIVWAGLPGQESGNSLVDVLYGRVSPGGKTPFT
WGKTRESYGAPLLTKPNNGKGAPQDDFTEGVFIDYRREDKYNETPIYEFGFGLSYTTFEY
SDIYVQPLNARPYTPASGSTKAAPTFGNISTDYADYLYPEDIHKVPLYIYPWLNTTDPKK
SSGDPDYGMKAEDYIPSGATDGSPQPILPAGGAPGGNPGLYDEMYRVSAIITNTGNVVGD
EVPQLYVSLGGPDDPKVVLRNEDRITLHPGQQTMWTTTLTRRDISNWDPASQNWVVTKYP
KTVYIGSSSRKLHLQAPLPPY

An exemplary enzyme having glycosidase (beta-glucosidase) activity is AoBGL1 from Aspergillus oryzae. The Aspergillus oryzae AoBGL1 is provided by the amino acid sequence set forth by SEQ ID NO: 16, which corresponds to UniProtKB Accession No. Q2UIR4 (gene 0090003001511).

(SEQ ID NO: 16)
MLTSPTARTSVRISRPATTERPNTVLTSGSLDIAMVQVVSRTLTPPTSNMKLSAALSTLA
ALQPAVGAAVQNRASDVADLEHYWSYGHSEPVYPTPETKGLGDWEEAFTKARSLVAQMTD
KEKNNITYGYSSTANGCGGTSGGVPRLGFPGICLQDAGNGVRGTDMVNSYASGVHVGASW
NRDLTYSRAQYMGAEFKRKGVNVALGPVAGPIGRIARGGRNWEGFSNDPYLSGALTGDTV
RGLQESVIACVKHLIGNEQETHRSTPSMLANSRNQSSSSNLDDKTMHELYLWPFQDAVKA
GAGSVMCSYNRINNSYGCQNSKAMNGLLKGELGFQGFVVSDWGAQHTGIASAAAGLDMAM
PSSSYWENGTLALAVKNESLPSTRLDDMATRIVATWYKYAEIENPGHGLPYSLLAPHNLT
DARDPKSKSTILQGAVEGHVLVKNTNNALPLKKPQFLSLFGYDAVAAARNTMDDLDWNMW
SMGYDNSLTYPNGSAVDAMMLKYIFLSSANPSAFGPGVALNATTITGGGSGASTASYIDA
PFNAFQRQAYDDDTFLAWDFASQNPLVNPASDACIVFINEQSSEGWDRPYLADPYSDTLV
QNVASQCSNIMVVIHNAGVRLVDRWIENDNITAVIYAHLPGQDSGRALVEVMYGKQSPSG
RLPYTVAKNESDYGSLLNPVIQSGTDDIYYPQDNFTEGVYIDYKAFVAANITPRYEFGYG
LTYSTFDYSDLKVSTSSNVSTSYLAPGTTVAEGGLPSVWDIIATVTCTVSNTGSVAAAEV
AQLYIGIPGGPAKVLRGFEKQLIEPGQQVQVTFDLTRRDLSTWDTEKQNWGLQAGSYALY
VGKSVLDIQLTGSLSL

An exemplary enzyme having glycosidase (beta-glucosidase) activity is AnBGL1 from Aspergillus niger (also referred to as BglA). The Aspergillus niger AnBGL1 is provided by the amino acid sequence set forth by SEQ ID NO: 17, which corresponds to AspGD systematic name An18g03570.

(SEQ ID NO: 17)
MRFTSIEAVALTAVSLASADELAYSPPYYPSPWANGQGDWAEAYQRAVDIVSQMTLAEKV
NLTTGTGWELELCVGQTGGVPRLGIPGMCAQDSPLGVRDSDYNSAFPAGVNVAATWDKNL
AYLRGQAMGQEFSDKGADIQLGPAAGPLGRSPDGGRNWEGFSPDPALSGVLFAETIKGIQ
DAGVVATAKHYIAYEQEHFRQAPEAQGYGFNITESGSANLDDKTMHELYLWPFADAIRAG
AGAVMCSYNQINNSYGCQNSYTLNKLLKAELGFQGFVMSDWAAHHAGVSGALAGLDMSMP
GDVDYDSGTSYWGTNLTISVLNGTVPQWRVDDMAVRIMAAYYKVGRDRLWTPPNFSSWTR
DEYGFKYYYVSEGPYEKVNQFVNVQRNHSELIRRIGADSTVLLKNDGALPLTGKERLVAL
IGEDAGSNPYGANGCSDRGCDNGTLAMGWGSGTANFPYLVTPEQAISNEVLKNKNGVFTA
TDNWAIDQIEALAKTASVSLVFVNADSGEGYINVDGNLGDRRNLTLWRNGDNVIKAAASN
CNNTIVIIHSVGPVLVNEWYDNPNVTAILWGGLPGQESGNSLADVLYGRVNPGAKSPFTW
GKTREAYQDYLYTEPNNGNGAPQEDFVEGVFIDYRGEDKRNETPIYEFGYGLSYTTENYS
NLQVEVLSAPAYEPASGETEAAPTFGEVGNASDYLYPDGLQRITKFIYPWLNSTDLEASS
GDASYGQDASDYLPEGATDGSAQPILPAGGGAGGNPRLYDELIRVSVTIKNTGKVAGDEV
PQLYVSLGGPNEPKIVLRQFERITLQPSKETQWSTTLTRRDLANWNVETQDWEITSYPKM
VFAGSSSRKLPLRASLPTVH*

An exemplary enzyme having glycosidase (beta-glucosidase) activity is TrBGL2 from Trichoderma reesei. The Trichoderma reesei TrBGL2 is provided by the amino acid sequence set forth by SEQ ID NO: 18, which corresponds to UniProtKB Accession No. AB003110.

(SEQ ID NO: 18)
MLPKDFQWGFATAAYQIEGAVDQDGRGPSIWDTFCAQPGKIADGSSGVTACDSYNRTAED
IALLKSLGAKSYRFSISWSRIIPEGGRGDAVNQAGIDHYVKFVDDLLDAGITPFITLFHW
DLPEGLHQRYGGLLNRTEFPLDFENYARVMFRALPKVRNWITFNEPLCSAIPGYGSGTFA
PGRQSTSEPWTVGHNILVAHGRAVKAYRDDFKPASGDGQIGIVLNGDFTYPWDAADPADK
EAAERRLEFFTAWFADPIYLGDYPASMRKQLGDRLPTFTPEERALVHGSNDFYGMNHYTS
NYIRHRSSPASADDTVGNVDVLFTNKQGNCIGPETQSPWLRPCAAGERDFLVWISKRYGY
PPIYVTENGTSIKGESDLPKEKILEDDFRVKYYNEYIRAMVTAVELDGVNVKGYFAWSLM
DNFEWADGYVTRFGVTYVDYENGQKRFPKKSAKSLKPLFDELIAAA

An exemplary enzyme having glycosidase (beta-glucosidase) activity is TrGL1 from Trichoderma reesei. The Trichoderma reesei TrGL1 is provided by the amino acid sequence set forth by SEQ ID NO: 19, which corresponds to UniProtKB Accession No. U09580.

(SEQ ID NO: 19)
MRYRTAAALALATGPFARADSHSTSGASAEAVVPPAGTPWGTAYDKAKAALAKLNLQDKV
GIVSGVGWNGGPCVGNTSPASKISYPSLCLQDGPLGVRYSTGSTAFTPGVQAASTWDVNL
IRERGQFIGEEVKASGIHVILGPVAGPLGKTPQGGRNWEGFGVDPYLTGIAMGQTINGIQ
SVGVQATAKHYILNEQELNRETISSNPDDRTLHELYTWPFADAVQANVASVMCSYNKVNT
TWACEDQYTLQTVLKDQLGFPGYVMTDWNAQHTTVQSANSGLDMSMPGIDENGNNRLWGP
ALTNAVNSNQVPTSRVDDMVTRILAAWYLTGQDQAGYPSFNISRNVQGNHKINVRAIARD
GIVLLKNDANILPLKKPASIAVVGSAAIIGNHARNSPSCNDKGCDDGALGMGWGSGAVNY
PYFVAPYDAINTRASSQGTQVTLSNTDNTSSGASAARGKDVAIVFITADSGEGYITVEGN
AGDRNNLDPWHNGNALVQAVAGANSNVIVVVHSVGAIILEQILALPQVKAVVWAGLPSQE
SGNALVDVLWGDVSPSGKLVYTIAKSPNDYNTRIVSGGSDSFSEGLFIDYKHFDDANITP
RYEFGYGLSYTKFNYSRLSVLSTAKSGPATGAVVPGGPSDLFQNVATVTVDIANSGQVTG
AEVAQLYITYPSSAPRTPPKQLRGFAKLNLTPGQSGTATFNIRRRDLSYWDTASQKWVVP
SGSFGISVGASSRDIRLTSTLSVA

An exemplary enzyme having glycosidase (beta-glucosidase) activity is BGL1 from Neurospora crassa. The Neurospora crassa BGL1 is provided by the amino acid sequence set forth by SEQ ID NO: 20, which corresponds to UniProtKB Accession No. EAA26868.1.

(SEQ ID NO: 20)
MKFAIPLALLASGNLALAAPEPIHPSHQQLNKRSLAYSEPHYPSPWMDPKAIGWEEAYEK
AKAFVSQLTLLEKVNLTTGIGWGAEQCVGQTGAIPRLGLKSMCMQDAPLAIRGTDYNSVF
PAGVTTAATFDRGLMYKRGYALGQEAKGKGVTVLLGPVAGPLGRAPEGGRNWEGESTDPV
LTGIAMAETIKGTQDAGVVACAKHFIGNEQEHFRQVGESQDYGYNISETLSSNIDDKTMH
EMYLWPFVDAIRAGVGSFMCAYTQANNSYSCQNSKLLNNLLKQENGFQGFVMSDWQAHHS
GVASAAAGLDMSMPGDTMFNSGRSYWGTNLTLAVLNGTVPQWRIDDMAMRIMAAFFKVGQ
TVEDQEPINFSFWTLDTYGPLHWAARKDYQQINWHVNVQGDHGSLIREIAARGTVLLKNT
GSLPLKKPKFLAVIGEDAGPNPLGPNGCADNRCNNGTLGIGWGSGTGNFPYLVTPDQALQ
ARAVQDGSRYESVLRNHAPTEIKALVSQQDATAIVFVNANSGEGFIEIDGNKGDRLNLTL
WNEGDALVKNVSSWCNNTIVVLHTPGPVLLTEWYDNPNITAILWAGMPGQESGNSITDVL
YGRVNPSGRTPFTWGATRESYGTDVLYEPNNGNEAPQLDYTEGVFIDYRHEDKANASVLY
EFGFGLSYTTFEYSNLKIEKHQVGEYTPTTGQTEAAPTFGNFSESVEDYVFPAAEFPYVY
QFIYPYLNSTDMSASSGDAQYGQTAEEFLPPKANDGSAQPLLRSSGLHHPGGNPALYDIM
YTVTADITNTGKVAGDEVPQLYVSLGGPEDPKVVLRGEDRLRVEPGEKVQFKAVLTRRDV
SSWDTVKQDWVITEYAKKVYVGPSSRKLDLEEVLP

An exemplary enzyme having glycosidase (beta-glucosidase) activity is BGL2 from Neurospora crassa. The Neurospora crassa BGL2 is provided by the amino acid sequence set forth by SEQ ID NO: 21, which corresponds to UniProtKB Accession No. EAA30164.1.

(SEQ ID NO: 21)
MHLRIFAVLAATSLAWAETSEKQARQAGSGFAAWDAAYSQASTALSKLSQQDKVNIVTGV
GWNKGPCVGNTPAIASIGYPQLCLQDGPLGIRFGGSVTAFTPGIQAASTWDVELIRQRGV
YLGAEARGVGVHVLLGPVAGALGKIPNGGRNWEGFGPDPYLTGIAMSETIEGIQSNGVQA
CAKHFILNEQETNRDTISSVVDDRIMHELYLFPFADAVHSNVASVMCSYNKVNGTWACEN
DKIQNGLLKKELGFKGYVMSDWNAQHTTNGAANSGMDMTMPGSDFNGKTILWGPQLNTAV
NNGQVSKARLDDMAKRILASWYLLEQNSGYPATNLKANVQGNHKENVRAVARDGIVLLKN
DDNILPLKKPSKLAIIGSSSVVNPAGRNACTDRGCNTGALGMGWGSGTADYPYFVAPYDA
LKTRAQSDGTTVNLLSSDSTSGVANAASGADAALVFITADSGEGYITVEGVTGDRPNLDP
WHNGNQLVQAVAQANKNTIVVVHSTGPIILETILAQPGVKAVVWAGLPSQENGNALVDVL
YGLVSPSGKLPYTIAKSESDYGTAVQRGGTDLFTEGLFIDYRHFDKNGIAPRYEFGFGLS
YTNFTYSSLSITSTASSGPASGDTIPGGRADLWETVATVTAVVKNTGGVQGAEAPQLYIT
LPSSAPSSPPKQLRGFAKLKLAPGESKTATFILRRRDLSYWDTGSQNWVVPSGSFGVVVG
ASSRDLRLNGKEDVY

An exemplary enzyme having glycosidase (beta-glucosidase) activity is bglH from Aspergillus oryzae. The Aspergillus oryzae bglH is provided by the amino acid sequence set forth by SEQ ID NO: 22, which corresponds to UniProtKB Accession No. BAE64214.1.

(SEQ ID NO: 22)
MLSPQALFGALVLLSAPSAVVADTCKAPINHPGEPFSFVQPLNTTILTPYGHSPPAYPSP
NTTGNGGWETALVKAKQWVNKLTLEEKTWMATGQPGPCVGNVLPIPRLNFSGICLQNGPQ
CVQQGDYSSVFVSSVSAAASWDRKLLYERAYALAEEHKAKGSHVILGPIGGPLGRSPYDG
RTWEGFAADPYLTGVCMEETINGMQDAGVQANAKHFIANEQETQRNPTYAPDANATTYIQ
DSVSANIDDRTLHEIYMWPFANAVRARVASAMCSYNRLNGSHSCQNSYLLNHLLKGELGF
QGYVMSDWGATHSGVASIESGMDMTMPGGFTLYGELWTEGSFFGKNLTEAVQNGTVPMSR
LDDMIVRIMTPYFWLGQEKNYPSVDASVGPLNVDSAPDTWLYDWKFTGATNRDVRANHSA
MIREHGGQSTVLLKNERNALPLRKPRNIVVAGNDAGPLTQGPDLQADFEYGVLAGSSGSG
SCRFSYLSTPLDAINARARKDGSLVQSYLNNTLLTTSALTSPLWIPQQPDVCLVFLKSES
AEGEDRTSLELDWNGNAVVEAVATHCNNTIVITNSGGANVMPFADHPNVTAILAQHYAGE
ETGNAIADVLYGDVNPSAKLPYVIAYNESDYNAPLTTAVQINGTYDWQSWFDEELEVGYR
YFDAHNISVRYEFGFGLSYTTFDLKDLKAKGSAAANLTALPAKRPTEPGGNPALWETVYT
LEAEVSNTGDVDGYAVPQLYLQFPTSTPAGTPPSQLRGFDKIWLEAGEKKTVTFDLMRRD
VSYWDVVAQDWRIPAGAFTFKAGFSSRDFRANSVATLVKA

An exemplary enzyme having glycosidase (beta-glucosidase) activity is OoBgl from Oenococcus oeni. The Oenococcus oeni OoBgl is provided by the amino acid sequence set forth by SEQ ID NO: 23, which corresponds to UniProtKB Accession No. EAV39986.1.

(SEQ ID NO: 23)
MSKITSIISGLSLKEKADLVSGKDFWFTAQVSGLDRMMVSDGPSGLRKQADASDALGLNK
SVVAVNFPSSSLTAASFDRALLQELGRNLGQAAKAERVGILLGPGINLKRSPLAGRNFEY
FSEDPYLTGELASSYVQGVQESGVGVSLKHFAANNREDQRFTASSNIDQRSLHELYLSAF
EKVVKMARPATIMCSYNAINGTLNSQNQRLLTQILREEWGFKGLVMSDWGAVSDHVAALK
AGLDLEMPGKGNESTSEIIEAVNKGQLDEKVLERAASRVIQMVEKWQPENKTVISYDLEK
QHRFARQLAGESIVLLKNEQQLLPLKSNQSLAVIGQLAEKPRYQGSGSAHVNAFNTTTPL
KVVQDILPKTAYQAGYQIDSDQIDQQAEQAAVDLAKQADQVVVFAGFPSSYESEGFDKKT
ISLPDNQNHLIERLAAVNKKIIVVLENGSALEMPWVGQVEAIVETYLAGEAVGEATWDIL
FGRVNPSGKLAESFPIKLADNPTYLTFNADRKNENYHEGLFVGYRYYDKKKQEVLFPFGH
GLSYTTFEYRKLELLKSDHEVTVSFEIKNTGSVAGKETAQIYLSNQTSEIEKPLKELKGF
AKVSLNPGQTKQVEIVLDKRSFSWYNPETDKWQVDNGSYQIQLAASSRDIRLTKNLLIDW
SENKRQALSPDSYLSDILKEQAFKAPLKESGLDKLLEQLAGDENNQAILTNMPLRALMMM
GVSNHQIQQFIKLANQS

An exemplary enzyme having glycosidase (beta-glucosidase) activity is OoBgl from Oenococcus oeni. The Oenococcus oeni OoBgl is provided by the amino acid sequence set forth by SEQ ID NO: 24, which corresponds to UniProtKB Accession No. WP_011677766.1.

(SEQ ID NO: 24)
MSKITSIISGLSLKEKADLVSGKDFWFTAQVSGLDRMMVSDGPSGLRKQADASNALGLNK
SVVAVNFPSSSLTAASFDRALLQELGRNLGQAAKAERVRILLGPGINLKRSPLAGRNFEY
FSEDPYLTGELASSYVQGVQESGVGVSLKRFAANNREDQRFTASSNIDQRSLHELYLSAF
EKAVKMARPATIMCSYNAINGTLNSQNQRLLTQILREEWGFKGLVMSDWGAVSDHVAALK
AGLDLEMPGKGNESTSEIIEAVNKGQLDEKVLERAASRVIQMVEKWQPENKTVISYDLEK
QHRFARQLVGESIVLLKNEQQLLPLKSNQSLAVIGQLAEKPRYQGSGSAHVNAFNTTTPL
KVVQDILPKTAYQAGYQIDSDQIDQQAEQAAVDLAKQADQVVVFSGFPSSYESEGEDKKT
ISLPDNQNHLIERLAAVNKKIIVVLENGSALEMPWVGQVEAIVETYLAGEAVGEATWDIL
FGRVNPSGKLAESFPIKLADNPTYLTFNADRKNENYHEGLFVGYRYYDKKKQEVLFPFGH
GLSYTTFEYRKLELLKSDHEVTVSFEIKNTGSVAGKETAQIYLSNQTSEIEKPLKELKGF
AKVSLNPGQTKQVEIVLDKRSFSWYNPETDKWQVDNGSYQIQLAASSRDIRLTKNLLIDW
SENKVQALSPDSYLSDILKEQAFKAPLKESGLDKLLEQLAGDENNQAILTNMPLRALMMM
GVSNHQIQQFIKLANQS

An exemplary enzyme having glycosidase (beta-glucosidase) activity is Ph691 from Prunus dulcis. The Prunus dulcis Ph69 is provided by the amino acid sequence set forth by SEQ ID NO: 25, which corresponds to UniProtKB Accession No. H9ZGE0.

(SEQ ID NO: 25)
MALQFRSLLLCMVLLLLGFALANTNAARTDPPIVCATLNRTHFDTLFPG
FTFGAATAAYQLEGAANIDGRGPSVWDNFTHEHPEKITDGSNGDVAIDQ
YHRYKEDVAIMKDMGLDAYRFSISWSRLLPNGKLSGGINKKGIEYYNNL
TNELLRNGIEPLVTLFHWDVPQALVDEYGGLLSPRIVDDFKAYADLCYK
EFGDRVKHWTTLNEPYTISNHGYTIGIHAPGRCSDWYNPKCLGGDSGIE
PYLVTHYLLLAHAAAVKLYREKYQAYQNGVIGITVVSHWFEPASESQQD
KDAAFQALDFMYGWFMDPLTRGDYPQIMRSILGARLPNFTEEQSKSLSG
SYDYIGVNYYSARYASAYPKDYSVTTPPSYLTDVHVNVTTDLNGVPIGP
RAASDWLYVYPKGLYDLVLYTKEKYNDPIMYITENGMDEFNNPKLSLEQ
ALNDANRIDYYYRHLCYLQAAMKEGANVQGYFAWSLLDNFEWSEGYTVR
FGINYIDYDNGLERHSKLSTHWFKSFLKRSSISKKKIRRCGNNNGRATK
FVYQI

An exemplary enzyme having glycosidase (beta-glucosidase) activity is Ph692 from Prunus dulcis. The Prunus dulcis Ph692 is provided by the amino acid sequence set forth by SEQ ID NO: 26, which corresponds to UniProtKB Accession No. H9ZGE2.

(SEQ ID NO: 26)
MAMQLRSLLLCVLLLLLGFALADTNAAARIHPPVVCANLSRANFDTLVP
GFVFGAATASYQVEGAANLDGRGPSIWDTFTHKHPEKIADGSNGDVAID
QYHRYKEDVAIMKDMGLESYRFSISWSRVLPNGTLSGGINKKGIEYYNN
LINELLHNGIEPLVTLFHWDVPQTLEDEYGGFLSNRIVNDFEEYAELCF
KKFGDRVKHWTTLNEPYTFSSHGYAKGTHAPGRCSAWYNQTCFGGDSAT
EPYLVTHNLLLAHAAAVKLYKTKYQAYQKGVIGITVVTPWFEPASEAKE
DIDAVFRALDFIYGWFMDPLTRGDYPQSMRSLVGERLPNFTKKESKSLS
GSFDYIGINYYSARYASASKNYSGHPSYLNDVNVDVKTELNGVPIGPQA
ASSWLYFYPKGLYDLLRYTKEKYNDPIIYITENGVDEFNQPNPKLSLCQ
LLDDSNRIYYYYHHLCYLQAAIKEGVKVKGYFAWSLLDNFEWDNGYTVR
FGINYVDYDNGLKRHSKHSTHWFKSFLKKSSRNTKKIRRCGNNNTSATK
FVF

An exemplary enzyme having glycosidase (beta-glucosidase) activity is BsBglA from Bacillus circulans. The Bacillus circulans BsBglA is provided by the amino acid sequence set forth by SEQ ID NO: 27, which corresponds to UniProtKB Accession No. Q03506.

(SEQ ID NO: 27)
MSIHMFPSDFKWGVATAAYQIEGAYNEDGRGMSIWDTFAHTPGKVKNGD
NGNVACDSYHRVEEDVQLLKDLGVKVYRFSISWPRVLPQGTGEVNRAGL
DYYHRLVDELLANGIEPFCTLYHWDLPQALQDQGGWGSRITIDAFAEYA
ELMFKELGGKIKQWITFNEPWCMAFLSNYLGVHAPGNKDLQLAIDVSHH
LLVAHGRAVTLFRELGISGEIGIAPNTSWAVPYRRTKEDMEACLRVNGW
SGDWYLDPIYFGEYPKFMLDWYENLGYKPPIVDGDMELIHQPIDFIGIN
YYTSSMNRYNPGEAGGMLSSEAISMGAPKTDIGWEIYAEGLYDLLRYTA
DKYGNPTLYITENGACYNDGLSLDGRIHDQRRIDYLAMHLIQASRAIED
GINLKGYMEWSLMDNFEWAEGYGMRFGLVHVDYDTLVRTPKDSFYWYKG
VISRGWLDL

An exemplary enzyme having glycosidase (beta-glucosidase) activity is SmBgl from Sinorhizobium meliloti. The Sinorhizobium meliloti SmBgl is provided by the amino acid sequence set forth by SEQ ID NO: 28, which corresponds to UniProtKB Accession No. F7X9K8.

(SEQ ID NO: 28)
MMIEAKKLAARFPGDFVFGVATASFQIEGASKADGRKASIWDAFSNMPG
RVYGRHNGDVACDHYNRLEQDLDLIKSLGVEAYRFSIAWPRIVPEGTGP
INEKGLDFYDRLVDGLKARGIKAFATLYHWDLPLALMGDGGWTARTTAY
AYQRYAKTVIARLGDRLDAVATFNEPWCSVWLGHLYGVHAPGERNMDAA
LAALHFTNLAHGLGVAAIRSERPELPVGIVINAHSVYPGSNSAEDKAAA
ERAFDFHNGVFFDPIFKGEYPEDFLSALGERMPAIEDGDMATIAQPLDW
WGLNYYTPMRVSADPAKGAEYPATVNAKPVSNVKTDIGWEVYAPALGSL
VETLNARYRLPDCYITENGACYNMGVENGTVDDQPRLDYISDHLAVTAD
LIAKGYPMRGYFAWSLMDNFEWAEGYRMRFGIVHVDYETQVRTIKKSGR
WYKDLAERFPSGNHKPG

In some embodiments, the heterologous gene encodes an enzyme with glycosidase activity such that a cell that expresses the enzyme is capable of increased production of volatile phenols as compared to a cell that does not express the heterologous gene. In some embodiments, the heterologous gene encodes an enzyme with glycosidase activity such that a cell that expresses the enzyme is capable of producing increased levels of volatile phenols as compared to a cell that expresses an enzyme with wild-type glycosidase activity.

In some embodiments, the enzyme with glycosidase activity (e.g., a recombinant and/or purified enzyme) is capable of increased production of volatile phenols in a fermented product as compared to a fermented product that is not contacted with the enzyme.

In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 1. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 2. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 3. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 4. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 5.

In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 6. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 7. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 8. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 9. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 10.

In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 11. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 12. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 13. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 14. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 15.

In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 16. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 17. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 18. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 19. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 20.

In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 21. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 22. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 23. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 24. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 25. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 26. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 27. In some embodiments, the enzyme with glycosidase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 28.

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). Percent identity can be determined using the algorithms of Karlin and Altschul, Proc. Natd. Acad. Sci. USA 87:2264-68, 1990, modified as in Karlin and Altschul, Proc. Natd. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%,at least 98%, at least 98.5%, at least 99%, at least 99.5%, at least 99.6%, at least 99.7%, at least 99.8%, or at least 99.9% identity) and all increments thereof (e.g., tenths of a percent (i.e., 0.1%), hundredths of a percent (i.e., 0.01%), etc.).

In some embodiments, the enzyme with glycosidase activity comprises the amino acid sequence as set forth in any one of SEQ ID NOs: 1-28. In some embodiments, the enzyme with glycosidase activity consists of the amino acid sequence as set forth in any one of SEQ ID NOs: 1-28.

In some embodiments, the gene encoding the enzyme with glycosidase activity comprises a nucleic acid sequence which encodes an enzyme comprising an amino acid sequence with at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%) sequence identity to the sequence as set forth in any one of SEQ ID NOs: 1-28. In some embodiments, the gene encoding the enzyme with glycosidase activity comprises a nucleic acid sequence which encodes an enzyme consisting of an amino acid sequence as set forth in any one of SEQ ID NOs: 1-28.

Identification of additional enzymes having glycosidase activity or predicted to have glycosidase activity may be performed, for example based on similarity or homology with one or more domains of a glycosidase, such as the glycosidases provided by any one of SEQ ID NOs: 1-28. In some embodiments, an enzyme for use in the modified cells and methods described herein may be identified based on similarity or homology with an active domain, such as a catalytic domain, such as a catalytic domain associated with glycosidase activity. In some embodiments, an enzyme for use in the modified cells and methods described herein may have a relatively high level of sequence identity with a reference glycosidase, e.g., a wild-type glycosidase, such as any one of SEQ ID NOs: 1-28, in the region of the catalytic domain but a relatively low level of sequence identity to the reference glycosidase based on analysis of a larger portion of the enzyme or across the full length of the enzyme. In some embodiments, the enzyme for use in the modified cells and methods described herein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity in the region of the catalytic domain of the enzyme relative to a reference glycosidase (e.g., SEQ ID NOs: 1-28).

In some embodiments, an enzyme for use in the modified cells and methods described herein has a relatively high level of sequence identity in the region of the catalytic domain of the enzyme relative to a reference glycosidase (e.g., SEQ ID NOs: 1-28) and a relatively low level of sequence identity to the reference glycosidase based on analysis of a larger portion of the enzyme or across the full length of the enzyme. In some embodiments, the enzymes for use in the modified cells and methods described herein have at least 10%, at least 15%, at least 20%, at least 25%, least 30% at least 35%, at least 40%, at least 45%, at least 50%, at least 55%, at least 60%, at least 65%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity based on a portion of the enzyme or across the full length of the enzyme relative to a reference glycosidase (e.g., SEQ ID NOs: 1-28).

In some embodiments, the enzyme with glycosidase activity is a recombinant and/or purified enzyme. Methods of producing a recombinant enzyme, isolating an enzyme, and purifying an enzyme (e.g., from a natural source or from a cell engineered to recombinantly express the enzyme) will be evident to one of ordinary skill in the art.

In some embodiments, the gene encoding the enzyme with glycosidase activity further comprises a secretion signal. The term “secretion signal,” as used herein, refers to a short peptide sequence (typically less than 70 amino acids) present at the terminus (N-terminus or C-terminus) of a newly synthesized protein that facilitates the export of the newly synthesized protein out of the cell. In some embodiments, the secretion signal facilitates the export of the glycosidase out of the cell. In some embodiments, the secretion signal is SED1, MATα, MATα pre-sequence (MATPRE), TFP5-1, TFP1-4, TFP10, TFP23, SUC2, SRL1, or KSH1. In some embodiments, the secretion signal is mutated at one or more amino acid residue positions. In some embodiments, the secretion signal comprises a substitution mutation at one or more amino acid residue positions. In some embodiments, the secretion signal is mutated relative to the wild-type secretion signal sequence. In some embodiments, the MATα secretion signal comprises a substitution mutation at amino acid residue position A9, A20, and/or L42, relative to the wild-type secretion signal sequence. In some embodiments, the MATα secretion signal comprises an A9D substitution mutation, relative to the wild-type secretion signal sequence. In some embodiments, the MATα secretion signal comprises an A20T substitution mutation, relative to the wild-type secretion signal sequence. In some embodiments, the MATα secretion signal comprises a L42S substitution mutation, relative to the wild-type secretion signal sequence. In some embodiments, the MATα secretion signal comprises an A9D substitution mutation and an A20T substitution mutation, relative to the wild-type secretion signal sequence. In some embodiments, the MATα secretion signal comprises an A9D substitution mutation, an A20T, and a L42S substitution mutation, relative to the wild-type secretion signal sequence. In some embodiments, the MATαPRE secretion signal comprises a substitution mutation at amino acid residue position A9 and/or A20, relative to the wild-type secretion signal sequence. In some embodiments, the MATαPRE secretion signal comprises an A9D substitution mutation, relative to the wild-type secretion signal sequence. In some embodiments, the MATαPRE secretion signal comprises an A20T substitution mutation, relative to the wild-type secretion signal sequence. In some embodiments, the MATαPRE secretion signal comprises an A9D substitution mutation and an A20T substitution mutation, relative to the wild-type secretion signal sequence.

An exemplary secretion signal is SED1, which is provided by the amino acid sequence set forth as SEQ ID NO: 29.

(SEQ ID NO: 29)
MKLSTVLLSAGLASTTLAQS

An exemplary secretion signal is MATα, which is provided by the amino acid sequence set forth as SEQ ID NO: 30.

(SEQ ID NO: 30)
MRFPSIFTAVLFAASSALAAPVNTTTEDETAQIPAEAVIGYLDLEGDED
VAVLPFSNSINNGLLFINTTIASIAAKEEGVSLDKREAEAS

An exemplary secretion signal is MATα pre-sequence, which is provided by the amino acid sequence set forth as SEQ ID NO: 31.

(SEQ ID NO: 31)
MRFPSIFTAVLFAASSALAS

An exemplary secretion signal is TFP5-1, which is provided by the amino acid sequence set forth as SEQ ID NO: 32.

(SEQ ID NO: 32)
MLQSVVFFALLTFASSVSAIYSNNTVSTTTTLAPSYSLVPQETTISYAD
DTTTFFVTSTLDKRS

An exemplary secretion signal is TFP1-4, which is provided by the amino acid sequence set forth as SEQ ID NO: 33.

(SEQ ID NO: 33)
MFNRFNKFQAAVALALLSRGALGDSYTNSTSSADLSSITSVSSASASAT
ASDSLSSSDGTVYLPSTTISGDLTVTGKVIATEAVEVAAGGKLTLLDGE
KYVFSSDLDKRS

An exemplary secretion signal is TFP10, which is provided by the amino acid sequence set forth as SEQ ID NO: 34.

(SEQ ID NO: 34)
MQYKKTLVASALAATTLAAYAPSEPWSTLTPTATYSGGVTDYASTFGIA
VQPISTTSSASSAATTASSKAKRAASQIGDGQVQAATTTASVSTKSTAA
AVSQIGDGQIQATTKTTAAAVSQIGDGQIQATTKTTSAKTTAAAVSQIS
DGQIQATTTTLAPKLDKRS

An exemplary secretion signal is TFP23, which is provided by the amino acid sequence set forth as SEQ ID NO: 35.

(SEQ ID NO: 35)
MFNRENKLQAALALVLYSQSALGQYYTNSSSIASNSSTAVSSTSSGSVS
ISSSIELTSSTSDVSSSLTELTSSSTEVSSSIAPSTSSSEVSSSITSSG
SSVSGSSSITSSLDKRS

An exemplary secretion signal is SUC2, which is provided by the amino acid sequence set forth as SEQ ID NO: 36.

(SEQ ID NO: 36)
MLLQAFLFLLAGFAAKISA

An exemplary secretion signal is SRL1, which is provided by the amino acid sequence set forth as SEQ ID NO: 50.

(SEQ ID NO: 50)
MLQSVVFFALLTFASSVSA

An exemplary secretion signal is KSH1, which is provided by the amino acid sequence set forth as SEQ ID NO: 51.

(SEQ ID NO: 51)
MSALFNERSLLQVILLLICSCSYVHG

An exemplary secretion signal is MATα(A9D;A20T), which is provided by the amino acid sequence set forth as SEQ ID NO: 52.

(SEQ ID NO: 52)
MRFPSIFTDVLFAASSALATPVNTTTEDETAQIPAEAVIGYLDLEGDED
VAVLPFSNSTNNGLLFINTTIASIAAKEEGVSLDKREAEAS

An exemplary secretion signal is MATα(A9D;A20T;L42S), which is provided by the amino acid sequence set forth as SEQ ID NO: 53.

(SEQ ID NO: 53)
MRFPSIFTDVLFAASSALATPVNTTTEDETAQIPAEAVIGYSDLEGDEDV
AVLPFSNSINNGLLFINTTIASIAAKEEGVSLDKREAEAS

An exemplary secretion signal is MATα pre-sequence (A9D;A20T), which is provided by the amino acid sequence set forth as SEQ ID NO: 54.

(SEQ ID NO: 54)
MRFPSIFTDVLFAASSALAT

O-methyltransferase (OMT) enzymes Aspects of the present related to modified cells comprising a gene encoding an enzyme having O-methyltransferase (OMT) activity. In some embodiments, the gene is a heterologous gene. In some embodiments, the modified yeast cells express a heterologous gene encoding an enzyme having glycosidase activity and a heterologous gene encoding an enzyme having O-methyltransferase (OMT) activity.

Aspects of the present disclosure also relate to contacting a medium or a fermented product with an enzyme having O-methyltransferase (OMT) activity. In some embodiments, the enzyme is a recombinant enzyme, e.g., that is produced by an organism, isolated, and/or purified and added to a medium or fermentation product. In some embodiments, the enzyme is extracted from a source, such as a microbial, yeast, mammalian, or plant source. Accordingly, any of the OMT enzymes described herein may be expressed as a heterologous enzyme in a genetically modified cell or provided in the form of a recombinant enzyme.

O-methyltransferases are enzymes that catalyze the methylation of acceptor molecules, or the transfer of a methyl group to the oxygen of an acceptor molecule. In some embodiments, the O-methyltransferase catalyzes the conversion of one or more volatile phenols to a methyl ether, thereby reducing or eliminating smoke taint in the fermented product. Following hydrolysis of a non-volatile phenolic glycoside by a glycosidase to produce a volatile phenol, the volatile phenols may be methylated by an O-methyltransferase to produce a methylated volatile phenol (also referred to as a methyl ether) that does not contribute the smoky off-flavor characteristic of smoke taint.

Several volatile phenols having relatively low sensory detection thresholds are guaiacol and m-cresol. Methylation of guaiacol generates veratrole (1,2-dimethoxybenzene), which acts as a pollinator attractor in various plants and has a sweet, creamy, vanilla odor. Methylation of m-cresol generates 3-methylanisole, which has a floral odor. In both cases, methylation would vastly improve the aroma profile of a smoke-tainted fermented beverage. Several enzymes capable of catalyzing these reactions have been characterized. For example, Gupta et al. identified and characterized an OMT from white campion that catalyzes the formation of veratrole (Gupta, et al., BMC Plant Biol. (2012) 12: 1-13). In addition, Hitschler et al. expressed an OMT from rose plant on a high copy plasmid in a lab yeast strain. When fed m-cresol, this strain generated 3-methylanisole, and after 72 hours, m-cresol was no longer detectable. (Hitschler, et al., FEMS Yeast Res. (2020) 20). In this case, methylation was intended as a tool to overcome the toxicity of m-cresol in order to improve microbial production of m-cresol for use in the pharmaceutical and chemical industries.

In some embodiments, any of the modified cells described herein are genetically modified to express a heterologous gene encoding an enzyme having O-methyltransferase activity. In some embodiments, the modified yeast cells express a heterologous gene encoding an enzyme having glycosidase activity and a heterologous gene encoding an enzyme having O-methyltransferase (OMT) activity. In some embodiments, the enzyme having O-methyltransferase activity is a recombinant enzyme (e.g., an isolated, purified enzyme).

In some embodiments, the heterologous gene encoding an enzyme with O-methyltransferase activity is a wild-type O-methyltransferase gene (e.g., a gene isolated from an organism). In some embodiments, the O-methyltransferase is obtained from a bacterium, a fungus, or a plant. In some embodiments, the O-methyltransferase is obtained from a fungus.

In some embodiments, the enzyme having O-methyltransferase activity is derived from Silene latifolia, Solanuma lycopersicum, Rosa hybrida, Ocimum basilicum, Eriobotrya japonica, Pinus taeda, Fragaria ananassa, Schizosaccharomyces pombe, or Zinnia elegans.

In some embodiments, the enzyme having O-methyltransferase activity is SIGOMT4 derived from Silene latifolia, SIOMT1 derived from Solanuma lycopersicum, SIOMT4 derived from Solanuma lycopersicum, SICTOMT1 derived from Solanuma lycopersicum, RhOOMT1 derived from Rosa hybrida, RhOOMT2 derived from Rosa hybrida, EOMT1 derived from Ocimum basilicum, EjOMT1 derived from Eriobotrya japonica, AEOMT derived from Pinus taeda, FaOMT derived from Fragaria ananassa, SpCOMT derived from Schizosaccharomyces pombe, COMT1 derived from Ocimum basilicum, or ZeCAOMT derived from Zinnia elegans.

An exemplary enzyme having O-methyltransferase activity is SIGOMT1 from Silene latifolia (white campion). The Silene latifolia SIGOMT1 is provided by the amino acid sequence set forth by SEQ ID NO: 37.

(SEQ ID NO: 37)
MENPKELLNAQAHIWNHIFAYHSSAALKCAIELGIPDTIEKHGNPMTLQD
LANSLAITPTKTLSLYRLLRLLVHSNFFSMTKLVDGEEAYANNINSQLLL
KDHPCTLAPFTLGMLDPAMTEPPHYLSKWFQNQDESVFHVIHGRSFWEHA
GLTPGFNQLFNRAMGSDASFVSIALVANKDFAKMVEGIGSLVDVAGGDGT
VAKIIARAYPWLKCTVFDLPQVVDGLQGNGSNLEYVAGDMFKEIPSADVV
MLKWILHDWSDEHCVRILERCKEAIPSNGKIIIIDMVVDPQAQNNNHFHA
QLLSDMEMMALNVGGIERTEDQWKKLFLQAGFNHYNIFPILGIRSVIEVR
CL

An exemplary enzyme having O-methyltransferase activity is SIOMT1 from Solanum lycopersicum (tomato). The Solanum lycopersicum SIOMT1 is provided by the amino acid sequence set forth by SEQ ID NO: 38, which corresponds to GenBank Accession No. MW380256.

(SEQ ID NO: 38)
MSSSESTSSELLHAQAQIWNYIFNFISSSAVRCAFQLGIPDVLYKHDKPM
CLSDISAELSVVNSSKVSFLPILMQFLVQSGFLNQHEDHYSLTPASCLLA
KDDPFNVRSLLLLNHGQAFSKAWPELSDWFQNDSPTPFHTAHGKSLWDFI
GEEQPSVLGDIFNDALASDSRLNTNVLIAECKHVFEGLTSLVDVGGGTGT
VSIAIAKAFPNIKCTVLDLPQVVGDLKGSGNLDFVGGDMEDMIPHTNAIL
LKCVLHDWNDEDCVKVLKKCKESIPSREKGGKVIIIDTVLEDPKQSNEFV
RAQHNMGMLMMVLFAAKERTEKEWEKLFSEAGFTEYKIFPALGLRSLIET
YP

An exemplary enzyme having O-methyltransferase activity is SIOMT4 from Solanum lycopersicum (tomato). The Solanum lycopersicum SIOMT4 is provided by the amino acid sequence set forth by SEQ ID NO: 39, which corresponds to GenBank Accession No. MW380257.

(SEQ ID NO: 39)
METNNNVERANELFKAQAHIYKHAFAYANSMALNCAIQLGIPDIIHNHKK
PITLPDLLSGLKLPSSKSNAIHRLMRLLVHAQFFDIIKLEENSETEGYVL
TTSSRLLLKSEIPNLLPCVRLMVDPVLVTPWQLLGEWFHKNEEATPFETA
HGMPMWDFCAQNPIFDTAFNEAMASDSQMMKLVVKDCREVFEGLNSLVDV
GGGTGVIAKTILEAIPHLKCTVLDLPHVVANMPQTENLIYVGGNMFQCIP
HADAILLKHVMHDWSDEDCVKILKRCREATEDKDEGRKGKVLIIDMVLGR
DEEEANMTEVKLIFDVLMMVVTTGRQRTEKEWEKLFTEAGFMSYKITPLL
GLRSLIQVFP

An exemplary enzyme having O-methyltransferase activity is SICTOMT1 from Solanum lycopersicum (tomato). The Solanum lycopersicum SICTOMT1 is provided by the amino acid sequence set forth by SEQ ID NO: 40, which corresponds to Sol Genomics Network Accession No. SGN-U582403.

(SEQ ID NO: 40)
MGSTANIQLATQSEDEERNCTYAMQLLSSSVLPFVLHSTIQLDVEDILAK
DKAATKLSALEIVSHMPNCKNPDAATMLDRMLYVLASYSLLDCSVVEEGN
GVTERRYGLSRVGKFFVRDEDGASMGPLLALLQDKVFINSWFELKDAVLE
GGVPFDRVHGVHAFEYPKLDPKENDVFNQAMINHTTVVMKRILENYKGFE
NLKTLVDVGGGLGVNLKMITSKYPTIKGTNFDLPHVVQHAPSYPGVDHVG
GDMFESVPQGDAIFMKWILHDWSDGHCLKLLKNCHKALPDNGKVIVVEAN
LPVKPDTDTTVVGVSQCDLIMMAQNPGGKERSEQEFRALASEAGFKGVNL
ICCVCNFWVMEFYK

An exemplary enzyme having O-methyltransferase activity is RhOOMT1 from Rosa hybrida (rose). The Rosa hybrida RhOOMT1 is provided by the amino acid sequence set forth by SEQ ID NO: 41, which corresponds to UniProtKB Accession No. AF502433.1.

(SEQ ID NO: 41)
MERLNSFRHLNQKWSNGEHSNELLHAQAHIWNHIFSFINSMSLKSAIQLG
IPDIINKHGYPMTLSELTSALPIHPTKSHSVYRLMRILVHSGFFAKKKLS
KTDEEGYTLTDASQLLLKDHPLSLTPYLTAMLDPVLTNPWNYLSTWFQND
DPTPFDTAHGMTFWDYGNHQPSIAHLENDAMASDARLVTSVIINDCKGVF
EGLESLVDVGGGTGTLAKAIADAFPHIECTVLDLPHVVADLQGSKNLKYT
GGDMFEAVPPADTVLLKWILHDWSDEECIKILERSRVAITGKEKKGKVII
IDMMMENQKGDEESIETQLFFDMLMMALVGGKERNEKEWAKLFTDAGFSD
YKITPISGLRSLIEVYP

An exemplary enzyme having O-methyltransferase activity is RhOOMT2 from Rosa hybrida (rose). The Rosa hybrida RhOOMT2 is provided by the amino acid sequence set forth by SEQ ID NO: 42, which corresponds to UniProtKB Accession No. AF502434.

(SEQ ID NO: 42)
MERLNSFKHLNQKWSNGEHSNELLHAQAHIWNHIFSFINSMSLKSAIQLG
IPDIINKHGPMTLSELTSALPIHPTKSHSVYRLMRILVHSGFFAKKKLSK
TDEEGYTLTDASQLLLKDHPLSLTPFLTAMLDPVLTTPWNYLSTWFQNED
PTPFDTAHGMTFWDYGNHQPSIAHLENDAMASDARLVTSVIIDDCKGVFE
GLESLVDVGGGTGTVAKAIADAFPHIECTVLDLPHVVADLQGSKNLKYTG
GDMFEAVPPADTVLLKWILHDWNDEECIKILKRSRVAITSKDKKGKVIII
DMMMENQKGDEESIETQLFFDMLMMALVRGQERNEKEWAKLFTDAGFSDY
KITPILGLRSLIEVYP

An exemplary enzyme having O-methyltransferase activity is EOMT1 from Ocimum basilicum (basil). The Ocimum basilicum EOMT1 is provided by the amino acid sequence set forth by SEQ ID NO: 43, which corresponds to UniProtKB Accession No. AF435008.

(SEQ ID NO: 43)
MALQKVDISLSTEQLLQAQVHVWNHMYAFANSMSLKCAIQLGIPDILHKH
GRPMTLSQLLQSIPINKEKTQCFQRLMRALVNSNFFIEENNSNNQEVCYW
LTPASCLLLKEAPLTVTPLVQVVLDPTFTNPWHHMSEWFTHEKHATQFEA
ANGCTFWEKLANEPSKGRFFDEAMSCDSRLIAHVFTKDYKHVIEGIRTLV
DVGGGNGTMAKAIVEAMPTIKCTVIDLPHVVAGLESTDNLNYIGGDMFQS
IPSADAILLKSIIHDWDDVEGLKILKKCKDAVVMGGKVIIIDVVVGVNHD
IDEVLEDQLHFDMAMMCYFNAKERTMSEWEKLIYDAGFKSYKLTPAFGVR
SLIEAYP

An exemplary enzyme having O-methyltransferase activity is EjOMT1 from Eriobotrya japonica (loquat). The Eriobotrya japonica EjOMT1 is provided by the amino acid sequence set forth by SEQ ID NO: 44, which corresponds to UniProtKB Accession No. LC127201.

(SEQ ID NO: 44)
MGLSNGDEVGATSHELLGAQAQIWNHMFQFINSMALKCAVQLGIPDVIHN
HGQPISLSELIAALNVHPSKAHFVSRLMRILVHSDFFAQHHHVHHDCDDV
EEEEAVVLYSLTPTSRLLLKDGPSNTTPLVLMILDPVLTTPFHLMGAWLQ
MNGGDDPATICTPFEMENGMPFWDLAAQEPRFGNLFDEAMEADSKLLGRE
VVEECGGVFEGLKSLVDVGGGTGTMAKAIANAFPSINCIVFDQPHVVADL
QGTTHNLGFVGGDMFVEIPPANAILLKWILHDWSDEESVKILKNCREAIL
LSKNNEGNKKIIIIDIVVGHVDNKEKMVDKKSIETQLMEDMLMMSTVTGK
ERSESEWKKIFLAAGFTHYNITHAFGERSLIELYPSK

An exemplary enzyme having O-methyltransferase activity is AEOMT from Pinus taeda (loblolly pine). The Pinus taeda AEOMT is provided by the amino acid sequence set forth by SEQ ID NO: 45, which corresponds to UniProtKB Accession No. U39301.

(SEQ ID NO: 45)
MDSNMNGLAKSNGCEISRDGFFESEEEELQGQAEAWKCTFAFAESLAVKC
VVLLGIPDMIAREGPRATLSLCEIVANLPTESPDAACLFRIMRELVAKGI
FPASKSARRRAFETRYGLTPASKWLVKGRELSMAPMLLMQNDETTLAPWH
HFNECVLEGGVAFQKANGAEIWSYASDHPDFNNLFNNAMACNARIVMKAI
LSKYQGFHSLNSLVDVGGGTGTAVAEIVRAYPFIRGINYDLPHVVATASS
LSGVQHVGGDMFETVPTADAIFMKWIMHDWNDEDCIKILKNCRKAIPDTG
KVIIVDVVLDADQGDNTDKKRKKAVDPIVGTVFDLVMVAHSSGGKERTEK
EWKRILLEGGFSRYNIIEIPALQSVIEAFPR

An exemplary enzyme having O-methyltransferase activity is FaOMT from Fragaria ananassa (strawberry). The Fragaria ananassa FaOMT is provided by the amino acid sequence set forth by SEQ ID NO: 46, which corresponds to UniProtKB Accession No. AF220491.2.

(SEQ ID NO: 46)
MGSTGETQMTPTHVSDEEANLFAMQLASASVLPMVLKAAIELDLLEIMAK
AGPGSFLSPSDLASQLPTKNPEAPVMLDRMLRLLASYSILTCSLRTLPDG
KVERLYCLGPVCKELTKNEDGVSIAALCLMNQDKVLVESWYHLKDAVLDG
GIPFNKAYGMTAFDYHGTDPRFNKVENKGMADHSTITMKKILETYKGFEG
LKSIVDVGGGTGAVVNMIVSKYPSIKGINFDLPHVIEDAPQYPGVQHVGG
DMFVSVPKGNAIFMKWICHDWSDEHCIKFLKNCYAALPDDGKVILAECIL
PVAPDTSLATKGVVHMDVIMLAHNPGGKERTEQEFEALAKGSGFQGIRVC
CDAFNTYVIEFLKKI

An exemplary enzyme having O-methyltransferase activity is SpCOMT from Schizosaccharomyces pombe. The Schizosaccharomyces pombe SpCOMT is provided by the amino acid sequence set forth by SEQ ID NO: 47, which corresponds to UniProtKB Accession No. NP_595284.1.

(SEQ ID NO: 47)
MPHMEDNGSEKEQLFLQHIQNLPQERLDAIRGHPELVLKEIDEFTYPDGS
GVRMCIGDVKGGFIVGKIRERKPKIMVELGGYLGYSAILFGNEISKIPGG
RYYSLEVNEDYAKIAYELVKLAGLDEIVTIMIGKACDSLVELQQKLLHKD
LGFQALDMVFIDHWKDLYVPDLRVIESLNMIAPGTLLVADNIITPGAPEY
HKYVNMSPEERRGYQAKVRNVNGEDFIGRWDLIYKTETKEFEGVIRNKHR
KDAVDVTECVGYAKKD

An exemplary enzyme having O-methyltransferase activity is COMT1 from Ocimum basilicum (basil). The Ocimum basilicum COMT1 is provided by the amino acid sequence set forth by SEQ ID NO: 48, which corresponds to UniProtKB Accession No. AAD38189.

(SEQ ID NO: 48)
MGSAINTPQINSDEEENFLFAMQLASASVLPMVLKSAIELDLLELIKKSG
AGAFVSPVDLAAQLPTTNPDAHVMLDRILRLLTSYAILECRLKTLPDGGV
ERLYGLAPVCKELTKNEDGVSMAPLTLMNQDKVLMESWYHLSDAVVDGGI
PENKAYGMTAFEYHGTDPRFNKVFNQGMSNHSTITMKKILETYTGFDGLK
TVVDVGGGTGATLNMIVSKYPSIKGINFDLPHVIEDAPSYPGVEHVGGDM
FVSVPKGDAIFMKWICHDWSDEHCVKFLKNCYDALPQNGKVILAECVLPE
APDTGLATKNVVHIDVIMLAHNPGGKERTEKEFQGLAKAAGFKQFNKACC
AYNTWIMELLK

An exemplary enzyme having O-methyltransferase activity is ZeCAOMT from Zinnia elegans (zinnia). The Zinnia elegans ZeCAOMT is provided by the amino acid sequence set forth by SEQ ID NO: 49, which corresponds to UniProtKB Accession No. U19911.

(SEQ ID NO: 49)
MGSNQDDQAFLFAMQLASASVLPMVLKTAIELDLLETIAKAGPHGSVSSS
ELVAQLPKVNNPEAPVMIDRICSLLASYSVLTCTLKETADGCAERFYGLA
PVCKFLIKNDAGVSLAPLLLMNQDKVLMESWYYLKDPVLDGGIPFNKAYG
MSAFEYHGKDQRENKVENSGMFNHSTMTMKKIVELYNGFSGLKTLVDVGG
GTGASLNMITSKHKSLKGINFDLPHVIADATTYQGIEHVGGDMFESVPKG
DAIFMKWILHDWSDAHCLQVLKNCYKSLPENGKVIVAECILPEAPDTTPA
TQNVIHIDVIMLAHNPGGKERTEKEFEALAKGAGFKGENKAACALNTWVM
E

In some embodiments, the heterologous gene encodes an enzyme with O-methyltransferase activity such that a cell that expresses the enzyme is capable of increased conversion of volatile phenols to methyl ethers. In some embodiments, the heterologous gene encodes an enzyme with O-methyltransferase activity such that a cell that expresses the enzyme is capable of increased conversion of volatile phenols to methyl ethers as compared to a cell that expresses an enzyme with wild-type O-methyltransferase activity. In some embodiments, the heterologous gene encodes an enzyme with O-methyltransferase activity such that a cell that expresses the enzyme is capable of increased conversion of volatile phenols to methyl ethers as compared to a cell that does not express the heterologous gene.

In some embodiments, the enzyme having OMT activity is a recombinant enzyme which can convert a volatile phenol to a phenolic methyl ether. In some embodiments, a fermented product contacted with an enzyme with O-methyltransferase activity has increased conversion of volatile phenols to methyl ethers as compared to fermented products that are not contacted with an OMT enzyme. In some embodiments, any of the methods described herein may further comprise adding one or more cofactors to the fermentation process and/or the medium to promote activity of the enzymes. In some embodiments, the methods further comprise adding exogenous S-adenosyl methionine to the fermentation process and/or to the medium. In some embodiments, the medium comprises S-adenosyl methionine.

Examples of methyl ethers (methylated volatile phenols) include, without limitation, veratrole (1,2-dimethoxybenzene), 3-methylanisole, 2,4,6-trimethylphenol, 2-methylphenol, 2,6-xylenol, 2,3,6-trimethylphenol, 2-methylanisole, and 4-methylanisole.

In some embodiments, the methyl ether is veratrole (1,2-dimethoxybenzene), 3-methylanisole, and/or 4-methylanisole.

In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 37. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 38. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 39. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 40.

In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 41. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 42. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 43. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 44. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 45.

In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 46. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 47. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 48. In some embodiments, the enzyme with O-methyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 49.

In some embodiments, the enzyme with O-methyltransferase activity comprises the amino acid sequence as set forth in any one of SEQ ID NOs: 37-49. In some embodiments, the enzyme with O-methyltransferase activity consists of the amino acid sequence as set forth in any one of SEQ ID NOs: 37-49.

In some embodiments, the gene encoding the enzyme with O-methyltransferase activity comprises a nucleic acid sequence which encodes an enzyme comprising an amino acid sequence with at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%) sequence identity to the sequence as set forth in any one of SEQ ID NOs: 37-49. In some embodiments, the gene encoding the enzyme with O-methyltransferase activity comprises a nucleic acid sequence which encodes an enzyme consisting of an amino acid sequence as set forth in any one of SEQ ID NOs: 37-49.

Identification of additional enzymes having O-methyltransferase activity or predicted to have O-methyltransferase activity may be performed, for example based on similarity or homology with one or more domains of a O-methyltransferase, such as the O-methyltransferase provided by any one of SEQ ID NOs: 37-49. In some embodiments, an enzyme for use in the modified cells and methods described herein may be identified based on similarity or homology with an active domain, such as a catalytic domain, such as a catalytic domain associated with O-methyltransferase activity. In some embodiments, an enzyme for use in the modified cells and methods described herein may have a relatively high level of sequence identity with a reference O-methyltransferase, e.g., a wild-type O-methyltransferase, such as any one of SEQ ID NOs: 37-49, in the region of the catalytic domain but a relatively low level of sequence identity to the reference O-methyltransferase based on analysis of a larger portion of the enzyme or across the full length of the enzyme. In some embodiments, the enzyme for use in the modified cells and methods described herein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity in the region of the catalytic domain of the enzyme relative to a reference O-methyltransferase (e.g., SEQ ID NOs: 37-49).

In some embodiments, an enzyme for use in the modified cells and methods described herein have a relatively high level of sequence identity in the region of the catalytic domain of the enzyme relative to a reference O-methyltransferase (e.g., SEQ ID NOs: 37-49) and a relatively low level of sequence identity to the reference O-methyltransferase based on analysis of a larger portion of the enzyme or across the full length of the enzyme. In some embodiments, the enzymes for use in the modified cells and methods described herein have at least 10%, at least 15%, at least 20%, at least 25%, least 30% at least 35%, at least 40%, at least 45%, at least 50%, at least 55%, at least 60%, at least 65%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity based on a portion of the enzyme or across the full length of the enzyme relative to a reference O-methyltransferase (e.g., SEQ ID NOs: 37-49).

### General Methods of Genetic Engineering

As will also be evident to one of ordinary skill in the art, the amino acid position number of a selected residue in a glycosidase and/or O-methyltransferase enzyme may have a different amino acid position number as compared to another glycosidase and/or O-methyltransferase enzyme (e.g., a reference enzyme). Generally, one may identify corresponding positions in other glycosidase and/or O-methyltransferase enzymes using methods known in the art, for example by aligning the amino acid sequences of two or more enzymes. Software programs and algorithms for aligning amino acid (or nucleotide) sequences are known in the art and readily available, e.g., Clustal Omega (Sievers et al. 2011).

The glycosidase and/or O-methyltransferase enzymes described herein may further contain one or more modifications, for example to specifically alter a feature of the polypeptide unrelated to its desired physiological activity. Alternatively or in addition, the glycosidase and/or O-methyltransferase enzymes described herein may contain one or more mutations to modulate expression and/or activity of the enzyme in the cell.

Mutations of a nucleic acid which encodes a glycosidase and/or O-methyltransferase preferably preserve the amino acid reading frame of the coding sequence, and preferably do not create regions in the nucleic acid which are likely to hybridize to form secondary structures, such a hairpins or loops, which can be deleterious to expression of the enzyme.

Mutations can be made by selecting an amino acid substitution, or by random mutagenesis of a selected site in a nucleic acid which encodes the polypeptide. As described herein, variant polypeptides can be expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host (referred to as codon optimization). The preferred codons for translation of a nucleic acid in, e.g., S. cerevisiae, are well known to those of ordinary skill in the art. Still other mutations can be made to the noncoding sequences of a gene or cDNA clone to enhance expression of the polypeptide. The activity of a glycosidase and/or O-methyltransferase (enzyme) variant can be tested by cloning the gene encoding the enzyme variant into an expression vector, introducing the vector into an appropriate host cell, expressing the enzyme variant, and testing for a functional capability of the enzyme, as disclosed herein.

The glycosidase and/or O-methyltransferase enzymes described herein may contain an amino acid substitution of one or more positions corresponding to a reference glycosidase and/or O-methyltransferase, such as a wild-type enzyme. In some embodiments, the glycosidase enzyme contains an amino acid substitution at 1, 2, 3, 4, 5, or more positions corresponding to a reference glycosidase. In some embodiments, the glycosidase is not a naturally occurring glycosidase, e.g., is genetically modified. In some embodiments, the O-methyltransferase enzyme contains an amino acid substitution at 1, 2, 3, 4, 5, or more positions corresponding to a reference O-methyltransferase. In some embodiments, the O-methyltransferase is not a naturally occurring O-methyltransferase, e.g., is genetically modified.

In some embodiments, the glycosidase and/or O-methyltransferase variant may also contain one or more amino acid substitutions that do not substantially affect the activity and/or structure of the glycosidase and/or O-methyltransferase enzyme. The skilled artisan will also realize that conservative amino acid substitutions may be made in the enzyme to provide functionally equivalent variants of the foregoing polypeptides, i.e., the variants retain the functional capabilities of the polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Exemplary functionally equivalent variants of polypeptides include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

As one of ordinary skill in the art would be aware, homologous genes encoding an enzyme having glycosidase and/or O-methyltransferase activity could be obtained from other species and could be identified by homology searches, for example through a protein BLAST search, available at the National Center for Biotechnology Information (NCBI) internet site (ncbi.nlm.nih.gov). By aligning the amino acid sequence of an enzyme with one or more reference enzymes and/or by comparing the secondary or tertiary structure of a similar or homologous enzyme with one or more reference eta lyase, one can determine corresponding amino acid residues in similar or homologous enzymes and can determine amino acid residues for mutation in the similar or homologous enzyme.

Genes associated with the disclosure can be obtained (e.g., by PCR amplification) from DNA from any source of DNA which contains the given gene. In some embodiments, genes associated with the invention are synthetic, e.g., produced by chemical synthesis in vitro. Any means of obtaining a gene encoding the enzymes described herein are compatible with the modified cells and methods described herein.

The disclosure provided herein involves recombinant expression of genes encoding an enzyme having glycosidase and/or O-methyltransferase activity, functional modifications, and variants of the foregoing, as well as uses relating thereto. Homologs and alleles of the nucleic acids associated with the invention can be identified by conventional techniques. Also encompassed by the invention are nucleic acids that hybridize under stringent conditions to the nucleic acids described herein. The term “stringent conditions” as used herein refers to parameters with which the art is familiar. Nucleic acid hybridization parameters may be found in references which compile such methods, e.g., Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F.M. Ausubel, et al., eds., John Wiley & Sons, Inc., New York.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code. The invention also embraces codon optimization to suit optimal codon usage of a host cell.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as enzymatic activity. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

Mutations can be made by selecting an amino acid substitution, or by random mutagenesis of a selected site in a nucleic acid which encodes the polypeptide. As described herein, variant polypeptides can be expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host (referred to as codon optimization). The preferred codons for translation of a nucleic acid in, e.g., S. cerevisiae, are well known to those of ordinary skill in the art. Still other mutations can be made to the noncoding sequences of a gene or cDNA clone to enhance expression of the polypeptide.

In one aspect of the present disclosure, one or more of the genes associated with the invention is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes, and artificial chromosomes.

A cloning vector is one which is able to replicate autonomously or integrated in the genome in a host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., 0-galactosidase, luciferase, or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined or operably linked when they are covalently linked in such a way as to place the expression or transcription of the coding sequence under the influence or control of the regulatory sequences. If it is desired that the coding sequences be translated into a functional protein, two DNA sequences are said to be operably joined or operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes of the present disclosure is expressed in a cell, a variety of transcription control sequences (e.g., promoter/enhancer sequences) can be used to direct its expression. In some embodiments, each of the genes is operably linked to a promoter (e.g., each gene linked to a separate promoter). The promoter can be a native promoter, i.e., the promoter of the gene in its endogenous context, which provides normal regulation of expression of the gene. In some embodiments, the promoter can be constitutive, i.e., the promoter is unregulated allowing for continual transcription of its associated gene (e.g., an enzyme having glycosidase and/or O-methyltransferase activity). A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA (RNA). That heterologous DNA (RNA) may be placed under operable control of transcriptional elements to permit the expression of the heterologous DNA in the host cell. As one of ordinary skill in the art would appreciate, any of the enzymes described herein can also be expressed in other yeast cells, including yeast strains used for producing wine, mead, sake, cider, etc.

A nucleic acid molecule that encodes an enzyme having glycosidase activity and/or an enzyme having O-methyltransferase activity of the present disclosure can be introduced into a cell or cells using methods and techniques that are standard in the art. For example, nucleic acid molecules can be introduced by standard protocols such as transformation including chemical transformation and electroporation, transduction, particle bombardment, etc.

Expressing the nucleic acid molecule encoding the enzymes of the claimed invention also may be accomplished by integrating the nucleic acid molecule into the genome.

The incorporation of genes can be accomplished either by incorporation of the nucleic acid encoding the enzyme(s) into the genome of the yeast cell, or by transient or stable maintenance of the new nucleic acid encoding the enzyme(s) as an episomal element. In eukaryotic cells, a permanent, inheritable genetic change is generally achieved by introduction of the DNA into the genome of the cell.

The heterologous gene may also include various transcriptional elements required for expression of the encoded gene product (e.g., enzyme having glycosidase and/or enzyme having O-methyltransferase activity). For example, in some embodiments, the gene encoding the enzyme having glycosidase and/or enzyme having O-methyltransferase may be operably linked to a promoter. In some embodiments, the promoter is an inducible promoter. In some embodiments, the promoter is active during a particular stage of a fermentation process. For example, in some embodiments, peak expression from the promoter is during an early stage of the fermentation process, e.g., before >50% of the fermentable sugars have been consumed. In some embodiments, peak expression from the promoter is during a late stage of the fermentation process e.g., after 50% of the fermentable sugars have been consumed.

Conditions in the medium change during the course of the fermentation process, for example the availability of nutrients and oxygen tend to decrease over time during fermentation as sugar source and oxygen become depleted. Additionally, the presence of other factors, such as products produced by metabolism of the cells, may increase. In some embodiments, the promoter is regulated by one or more conditions in the fermentation process, such as presence or absence of one or more factors. In some embodiments, the promoter is regulated by hypoxic conditions. Examples of promoters of hypoxia activated genes are known in the art. See, e.g., Zitomer et al. Kidney Int. (1997) 51(2): 507-13; Gonzalez Siso et al. Biotechnol. Letters (2012) 34: 2161-2173.

In some embodiments, the promoter is a constitutive promoter. Examples of constitutive promoters for use in yeast cells are known in the art and evident to one of ordinary skill in the art. In some embodiments, the promoter is a yeast promoter, e.g., a native promoter from the yeast cell in which the heterologous gene or the exogenous gene is expressed.

Non-limiting examples of promoters for use in the genetically modified cells and methods described herein include, the HEM13 promoter (pHEM13), SPG1 promoter (pSPG1), PRB1 promoter (pPRB1), QCR10 (pQCR10), PGK1 promoter (pPGK1), OLE1 promoter (pOLE1), ERG25 promoter (pERG25), the HHF2 promoter (pHHF2), the TDH1 promoter (pTDH1), the TDH2 promoter (pTDH2), the TDH3 promoter (pTDH3), the ENO2 promoter (pENO2), or the HSP26 promoter (pHSP26).

### Genetically Modified Yeast Cells

Aspects of the present disclosure relate to genetically modified yeast cells (modified cells) and use of such modified cells in methods of producing a fermented product (e.g., a fermented beverage) and methods of producing ethanol. The genetically modified yeast cells described herein are genetically modified with a heterologous gene encoding an enzyme with glycosidase activity, and/or a gene encoding an enzyme with O-methyltransferase activity.

The terms “genetically modified cell,” “genetically modified yeast cell,” and “modified cell,” as may be used interchangeably herein, to refer to a eukaryotic cell (e.g., a yeast cell), which has been, or may be presently, modified by the introduction of a heterologous gene. The terms (e.g., modified cell) include the progeny of the original cell which has been genetically modified by the introduction of a heterologous gene. It shall be understood by the skilled artisan that the progeny of a single cell may not necessarily be completely identical in morphology or in genomic or total nucleic acid complement as the original parent, due to mutation (i.e., natural, accidental, or deliberate alteration of the nucleic acids of the modified cell).

Yeast cells for use in the methods described herein are preferably capable of fermenting a sugar source (e.g., a fermentable sugar) and producing ethanol (ethyl alcohol) and carbon dioxide. In some embodiments, the yeast cell is of the genus Saccharomyces. The Saccharomyces genus includes nearly 500 distinct species, many of which are used in food production. One example species is Saccharomyces cerevisiae (S. cerevisiae), which is commonly referred to as “brewer's yeast” or “baker's yeast,” and is used in the production of wine, bread, beer, among other products. Other members of the Saccharomyces genus include, without limitation, the wild yeast Saccharomyces paradoxus, which is a close relative to S. cerevisiae; Saccharomyces bayanus, Saccharomyces pastorianus, Saccharomyces carlsbergensis, Saccharomyces uvarum, Saccharomyces cerevisiae var boulardii, Saccharomyces eubayanus. In some embodiments, the yeast is Saccharomyces cerevisiae (S. cerevisiae).

Saccharomyces species may be haploid (i.e., having a single set of chromosomes), diploid (i.e., having a paired set of chromosomes), or polyploid (i.e., carrying or containing more than two homologous sets of chromosomes). Saccharomyces species used, for example for beer brewing, are typically classified into two groups: ale strains (e.g., S. cerevisiae), which are top fermenting, and lager strains (e.g., S. pastorianus, S. carlsbergensis, S. uvarum), which are bottom fermenting. These characterizations reflect their separation characteristics in open square fermentors, as well as other characteristics such as preferred fermentation temperatures and alcohol concentrations achieved.

Although beer brewing and wine producing has traditionally focused on use of S. cerevisiae strains, other yeast species and genera have been appreciated in production of fermented beverages. In some embodiments, the yeast cell belongs to a non-Saccharomyces genus. See, e.g., Crauwels et al. Brewing Science (2015) 68: 110-121; Esteves et al. Microorganisms (2019) 7(11): 478. In some embodiments, the yeast cell is of the genus Kloeckera, Candida, Starmerella, Hanseniaspora, Kluyveromyces/Lachance, Metschnikowia, Saccharomycodes, Zygosaccharomyces, Dekkera (also referred to as Brettanomyces), Wickerhamomyces, or Torulaspora. Examples of non-Saccharomyces yeast include, without limitation, Hanseniaspora uvarum, Hanseniaspora guillermondii, Hanseniaspora vinae, Metschnikowia pulcherrima, Kluyveromyces/Lachancea thermotolerans, Starmerella bacillaris (previously referred to as Candida stellata/Candida zemplinina), Saccharomycodes ludwigii, Zygosaccharomyces rouxii, Dekkera bruxellensis, Dekkera anomala, Brettanomyces custersianus, Brettanomyces naardenensis, Brettanomyces nanus, Wickerhamomyces anomalus, and Torulaspora delbrueckii.

In some embodiments, the methods described herein involve use of more than one genetically modified yeast. For example, in some embodiments, the methods may involve use of more than one genetically modified yeast belonging to the genus Saccharomyces. In some embodiments, the methods may involve use of more than one genetically modified yeast belonging to a non-Saccharomyces genus. In some embodiments, the methods may involve use of more than one genetically modified yeast belonging to the genus Saccharomyces and one genetically modified yeast belonging to a non-Saccharomyces genus. Alternatively, or in addition, any of the methods described herein may involve use of one or more genetically modified yeast and one or more non-genetically modified (wildtype) yeast.

In some embodiments, the yeast is a hybrid strain. As will be evident to one of ordinary skill in the art, the term “hybrid strain” of yeast refers to a yeast strain that has resulted from the crossing of two different yeast strains, for example, to achieve one or more desired characteristics. For example, a hybrid strain may result from the crossing of two different yeast strains belonging to the same genus or the same species. In some embodiments, a hybrid strain results from the crossing of a Saccharomyces cerevisiae strain and a Saccharomyces eubayanus strain. See, e.g., Krogerus et al. Microbial Cell Factories (2017) 16: 66.

In some embodiments, the yeast strain is a wild yeast strain, such as a yeast strain that is isolated from a natural source and subsequently propagated. Alternatively, in some embodiments, the yeast strain is a domesticated yeast strain. Domesticated yeast strains have been subjected to human selection and breeding to have desired characteristics.

In some embodiments, the genetically modified yeast cells may be used in symbiotic matrices with other yeast or bacterial strains. Symbiotic matrices of yeast cells and bacterial strains may be used, for example, for the production of fermented beverages, such as kombucha, kefir, and ginger beers. Saccharomycesfragilis, for example, is part of kefir culture and is grown on the lactose contained in whey. Other bacterial strains that may be used in symbiotic matrices with the genetically modified yeast cells include Bifidobacterium animalis subsp. lactis, Bifidobacterium breve, bacteria in the genus Lactobacillus, and bacteria in the genus Pediococcus.

Although many fermented beverages are produced using S. cerevisiae strains, other yeast genera have been appreciated in production of fermented beverages and may be used in symbiotic matrices with the modified yeast cells. In some embodiments, the other yeast cell belongs to a non-Saccharomyces genus. See, e.g., Crauwels et al. Brewing Science (2015) 68: 110-121; Esteves et al. Microorganisms (2019) 7(11): 478. In some embodiments, the other yeast cell is of the genus Kloeckera, Candida, Starmerella, Hanseniaspora, Kluyveromyces/Lachance, Metschnikowia, Saccharomycodes, Zygosaccharomyce, Dekkera (also referred to as Brettanomyces), Wickerhamomyces, or Torulaspora. Examples of non-Saccharomyces yeast include, without limitation, Hanseniaspora uvarum, Hanseniaspora guillermondii, Hanseniaspora vinae, Metschnikowia pulcherrima, Kluyveromyces/Lachancea thermotolerans, Starmerella bacillaris (previously referred to as Candida stellata/Candida zemplinina), Saccharomycodes ludwigii, Zygosaccharomyces rouxii, Dekkera bruxellensis, Dekkera anomala, Brettanomyces custersianus, Brettanomyces naardenensis, Brettanomyces nanus, Wickerhamomyces anomalus, and Torulaspora delbrueckii.

Methods of genetically modifying yeast cells are known in the art. In some embodiments, the yeast cell is diploid and one copy of a heterologous gene encoding an enzyme with glycosidase activity as described herein is introduced into the yeast genome.

In some embodiments, the yeast cell is diploid and one copy of a heterologous gene encoding an enzyme with glycosidase activity as described herein is introduced into both copies of the yeast genome. In some embodiments, the copies of the heterologous gene are identical. In some embodiments, the copies of the heterologous gene are not identical, but the genes encode an identical enzyme having glycosidase activity. In some embodiments, the copies of the heterologous gene are not identical, and the genes encode enzymes having glycosidase activity that are different (e.g., mutants, variants, fragments thereof). In some embodiments, the cell contains a gene encoding an enzyme with glycosidase activity, referred to as an endogenous gene, and also contains a second gene encoding an enzyme with glycosidase activity, which may be the same or different enzyme with glycosidase activity as that encoded by the endogenous gene.

In some embodiments, the yeast cell is diploid and one copy of a gene encoding an enzyme with O-methyltransferase activity as described herein is introduced into both copies of the yeast genome. In some embodiments, the copies of the gene encoding an enzyme with O-methyltransferase activity are identical. In some embodiments, the copies of the gene encoding an enzyme with O-methyltransferase activity are not identical, but the genes encode an identical enzyme having O-methyltransferase activity. In some embodiments, the copies of the gene encoding an enzyme with O-methyltransferase activity are not identical, and the genes encode enzymes having O-methyltransferase activity that are different (e.g., mutants, variants, fragments thereof). In some embodiments, the cell contains a gene encoding an enzyme with O-methyltransferase activity, referred to as an endogenous gene, and also contains a second gene encoding an enzyme with O-methyltransferase activity, which may be the same or different enzyme with O-methyltransferase activity as that encoded by the endogenous gene.

In some embodiments, the yeast cell is tetraploid. Tetraploid yeast cells are cells which maintain four complete sets of chromosomes (i.e., a complete set of chromosomes in four copies). In some embodiments, the yeast cell is tetraploid and a copy of a heterologous gene encoding an enzyme with glycosidase activity as described herein is introduced into at least one copy of the genome. In some embodiments, the yeast cell is tetraploid and a copy of a heterologous gene encoding an enzyme with glycosidase activity as described herein is introduced into more than one copy of the genome. In some embodiments, the yeast cell is tetraploid and a copy of a heterologous gene encoding an enzyme with glycosidase activity as described herein is introduced into all four copies of the genome. In some embodiments, the copies of the heterologous gene are identical. In some embodiments, the copies of the heterologous gene are not identical, but the genes encode an identical enzyme having glycosidase activity. In some embodiments, the copies of the heterologous gene are not identical, and the genes encode enzymes having glycosidase activity that are different (e.g., mutants, variants, fragments thereof).

In some embodiments, the yeast cell is tetraploid and a copy of a gene encoding an enzyme with O-methyltransferase activity as described herein is introduced into at least one copy of the genome. In some embodiments, the yeast cell is tetraploid and a copy of a gene encoding an enzyme with O-methyltransferase activity as described herein is introduced into more than one copy of the genome. In some embodiments, the yeast cell is tetraploid and a copy of a gene encoding an enzyme with O-methyltransferase activity as described herein is introduced into all four copies of the genome. In some embodiments, the copies of the gene encoding an enzyme with O-methyltransferase activity are identical. In some embodiments, the copies of the gene encoding an enzyme with O-methyltransferase activity are not identical, but the genes encode an identical enzyme having O-methyltransferase activity. In some embodiments, the copies of the gene encoding an enzyme with O-methyltransferase activity are not identical, and the genes encode enzymes having O-methyltransferase activity that are different (e.g., mutants, variants, fragments thereof). In some embodiments, the cell contains a gene encoding an enzyme with O-methyltransferase activity, referred to as an endogenous gene, and also contains one or more additional copies of a gene encoding an enzyme with O-methyltransferase activity, which may be the same or different enzyme with O-methyltransferase activity as that encoded by the endogenous gene.

In some embodiments, the growth rate of the modified cell is not substantially impaired relative to a wild-type yeast cell that does not comprise the first heterologous gene and second exogenous gene. Methods of measuring and comparing the growth rates of two cells will be known to one of ordinary skill in the art. Non-limiting examples of growth rates that can be measured and compared between two types of cells are replication rate, budding rate, colony-forming units (CFUs) produced per unit of time, and amount of fermentable sugar reduced in a medium per unit of time. The growth rate of a modified cell is “not substantially impaired” relative to a wild-type cell if the growth rate, as measured, is at least 70%, at least 75%, at least 80%, at least 85%, at least 90%, at least 95%, at least 99%, or at least 100% of the growth rate of the wild-type cell.

Strains of yeast cells that may be used with the methods described herein will be known to one of ordinary skill in the art and include yeast strains used for brewing desired fermented beverages as well as commercially available yeast strains. Examples of common beer strains include, without limitation, American ale strains, Belgian ale strains, British ale strains, Belgian lambic/sour ale strains, Barleywine/Imperial Stout strains, India Pale Ale strains, Brown Ale strains, Kolsch and Altbier strains, Stout and Porter strains, and Wheat beer strains.

Non-limiting examples of strains for use with the genetically modified cells and methods described herein include Wyeast American Ale 1056, Wyeast American Ale 111272, Wyeast Denny's Favorite 50 1450, Wyeast Northwest Ale 1332, Wyeast Ringwood Ale 1187, Siebel Inst. American Ale BRY 96, White Labs American Ale Yeast Blend WLP060, White Labs California Ale V WLP051, White Labs California Ale WLP001, White Labs Old Sonoma Ale WLP076, White Labs Pacific Ale WLPO41, White Labs East Coast Ale WLP008, White Labs East Midlands Ale WLP039, White Labs San Diego Super Yeast WLP090, White Labs San Francisco Lager WLP810, White Labs Neutral Grain WLP078, Lallemand American West Coast Ale BRY-97, Lallemand CBC-1 (Cask and Bottle Conditioning), Brewferm Top, Coopers Pure Brewers' Yeast, Fermentis US-05, Real Brewers Yeast Lucky #7, Muntons Premium Gold, Muntons Standard Yeast, East Coast Yeast Northeast Ale ECY29, East Coast Yeast Old Newark Ale ECY10, East Coast Yeast Old Newark Beer ECY12, Fermentis Safale US-05, Fermentis Safbrew T-58, Real Brewers Yeast The One, Mangrove Jack US West Coast Yeast, Mangrove Jack Workhorse Beer Yeast, Lallemand Abbaye Belgian Ale, White Labs Abbey IV WLP540, White Labs American Farmhouse Blend WLP670, White Labs Antwerp Ale WLP515, East Coast Yeast Belgian Abbaye ECY09, White Labs Belgian Ale WLP550, Mangrove Jack Belgian Ale Yeast, Wyeast Belgian Dark Ale 3822-PC, Wyeast Belgian Saison 3724, White Labs Belgian Saison I WLP565, White Labs Belgian Saison II WLP566, White Labs Belgian Saison III WLP585, Wyeast Belgian Schelde Ale 3655-PC, Wyeast Belgian Stout 1581-PC, White Labs Belgian Style Ale Yeast Blend WLP575, White Labs Belgian Style Saison Ale Blend WLP568, East Coast Yeast Belgian White ECY11, Lallemand Belle Saison, Wyeast Biere de Garde 3725-PC, White Labs Brettanomyces Bruxellensis Trois Vrai WLP648, Brewferm Top, Wyeast Canadian/Belgian Ale 3864-PC, Lallemand CBC-1 (Cask and Bottle Conditioning), Wyeast Farmhouse Ale 3726-PC, East Coast Yeast Farmhouse Brett ECY03, Wyeast Flanders Golden Ale 3739-PC, White Labs Flemish Ale Blend WLP665, White Labs French Ale WLP072, Wyeast French Saison 3711, Wyeast Leuven Pale Ale 3538-PC, Fermentis Safbrew T-58, East Coast Yeast Saison Brasserie Blend ECY08, East Coast Yeast Saison Single-Strain ECY14, Real Brewers Yeast The Monk, Siebel Inst. Trappist Ale BRY 204, East Coast Yeast Trappist Ale ECY13, White Labs Trappist Ale WLP500, Wyeast Trappist Blend 3789-PC, Wyeast British Ale 1098, Wyeast British Ale 111335, Wyeast British Cask Ale 1026-PC, Wyeast English Special Bitter 1768-PC, Wyeast Irish Ale 1084, Wyeast London Ale 1028, Wyeast London Ale III 1318, Wyeast London ESB Ale 1968, Wyeast Ringwood Ale 1187, Wyeast Thames Valley Ale 1275, Wyeast Thames Valley Ale II 1882-PC, Wyeast West Yorkshire Ale 1469, Wyeast Whitbread Ale 1099, Mangrove Jack British Ale Yeast, Mangrove Jack Burton Union Yeast, Mangrove Jack Workhorse Beer Yeast, East Coast Yeast British Mild Ale ECY18, East Coast Yeast Northeast Ale ECY29, East Coast Yeast Burton Union ECY17, East Coast Yeast Old Newark Ale ECY10, White Labs Bedford British Ale WLP006, White Labs British Ale WLP005, White Labs Burton Ale WLPO23, White Labs East Midlands Ale WLP039, White Labs English Ale Blend WLP085, White Labs English Ale WLP002, White Labs Essex Ale Yeast WLP022, White Labs Irish Ale WLP004, White Labs London Ale WLP013, White Labs Manchester Ale WLP038, White Labs Old Sonoma Ale WLP076, White Labs San Diego Super Yeast WLP090, White Labs Whitbread Ale WLP017, White Labs North Yorkshire Ale WLP037, Coopers Pure Brewers' Yeast, Siebel Inst. English Ale BRY 264, Muntons Premium Gold, Muntons Standard Yeast, Lallemand Nottingham, Fermentis Safale S-04, Fermentis Safbrew T-58, Lallemand Windsor (British Ale), Real Brewers Yeast Ye Olde English, Brewferm Top, White Labs American Whiskey WLP065, White Labs Dry English Ale WLP007, White Labs Edinburgh Ale WLPO28, Fermentis Safbrew S-33, Wyeast Scottish Ale 1728, East Coast Yeast Scottish Heavy ECY07, White Labs Super High Gravity WLP099, White Labs Whitbread Ale WLP017, Wyeast Belgian Lambic Blend 3278, Wyeast Belgian Schelde Ale 3655-PC, Wyeast Berliner-Weisse Blend 3191-PC, Wyeast Brettanomyces Bruxellensis 5112, Wyeast Brettanomyces Lambicus 5526, Wyeast Lactobacillus 5335, Wyeast Pediococcus Cerevisiae 5733, Wyeast Roeselare Ale Blend 3763, Wyeast Trappist Blend 3789-Pc, White Labs Belgian Sour Mix Wlp655, White Labs Berliner Weisse Blend Wlp630, White Labs Saccharomyces “Bruxellensis” Trois Wlp644, White Labs Brettanomyces Bruxellensis Wlp650, White Labs Brettanomyces Claussenii Wlp645, White Labs Brettanomyces Lambicus Wlp653, White Labs Flemish Ale Blend Wlp665, East Coast Yeast Berliner Blend Ecy06, East Coast Yeast Brett Anomala Ecy04, East Coast Yeast Brett Bruxelensis Ecy05, East Coast Yeast Brett Custersianus Ecy19, East Coast Yeast Brett Nanus Ecy16, Strain #2, East Coast Yeast BugCounty ECY20, East Coast Yeast BugFarm ECY01, East Coast Yeast Farmhouse Brett ECY03, East Coast Yeast Flemish Ale ECYO2, East Coast Yeast Oud Brune ECY23, Wyeast American Ale 1056, Siebel Inst. American Ale BRY 96, White Labs American Ale Yeast Blend WLP060, White Labs Bourbon Yeast WLP070, White Labs California Ale V WLP051, White Labs California Ale WLP001, White Labs Dry English ale WLP007, White Labs East Coast Ale WLP008, White Labs Neutral Grain WLP078, White Labs Super High Gravity WLP099, White Labs Tennessee WLP050, Fermentis US-05, Real Brewers Yeast Lucky #7, Fermentis Safbrew S-33, East Coast Yeast Scottish Heavy ECY07, Lallemand Windsor (British Ale), Wyeast American Ale 1056, Wyeast American Ale 111272, Wyeast British Ale 1098, Wyeast British Ale 111335, Wyeast Denny's Favorite 50 1450, Wyeast London Ale 1028, Wyeast London Ale III 1318, Wyeast London ESB Ale 1968, Wyeast Northwest Ale 1332, Wyeast Ringwood Ale 1187, Siebel Inst. American Ale BRY 96, White Labs American Ale Yeast Blend WLP060, White Labs Bedford British Ale WLP006, White Labs British Ale WLP005, White Labs Burton Ale WLPO23, White Labs California Ale V WLP051, White Labs California Ale WLP001, White Labs East Coast Ale WLP008, White Labs English Ale WLP002, White Labs London Ale WLP013, White Labs Essex Ale Yeast WLP022, White Labs Pacific Ale WLPO41, White Labs San Diego Super Yeast WLP090, White Labs Whitbread Ale WLP017, Brewferm Top, Mangrove Jack Burton Union Yeast, Mangrove Jack US West Coast Yeast, Mangrove Jack Workhorse Beer Yeast, Coopers Pure Brewers' Yeast, Fermentis US-05, Fermentis Safale S-04, Fermentis Safbrew T-58, Real Brewers Yeast Lucky #7, Real Brewers Yeast The One, Muntons Premium Gold, Muntons Standard Yeast, East Coast Yeast Northeast Ale ECY29, Lallemand Nottingham, Lallemand Windsor (British Ale), Wyeast American Ale 1056, Wyeast American Ale 111272, Wyeast British Ale 1098, Wyeast British Ale 111335, Wyeast Thames Valley Ale 1275, Wyeast Thames Valley Ale II 1882-PC, Wyeast West Yorkshire Ale 1469, Wyeast Whitbread Ale 1099, Wyeast British Cask Ale 1026-PC, Wyeast English Special Bitter 1768-PC, Wyeast London Ale 1028, Wyeast London Ale III 1318, Wyeast London ESB Ale 1968, Wyeast Northwest Ale 1332, Wyeast Ringwood Ale 1187, White Labs American Ale Yeast Blend WLP060, White Labs British Ale WLP005, White Labs Bedford British Ale WLP006, White Labs British Ale WLP005, White Labs Burton Ale WLPO23, White Labs California Ale V WLP051, White Labs California Ale WLP001, White Labs East Coast Ale WLP008, White Labs English Ale WLP002, White Labs Essex Ale Yeast WLP022, White Labs French Ale WLP072, White Labs London Ale WLP013, White Labs Pacific Ale WLPO41, White Labs Whitbread Ale WLP017, Brewferm Top, East Coast Yeast British Mild Ale ECY18, Coopers Pure Brewers' Yeast, Muntons Premium Gold, Muntons Standard Yeast, Mangrove Jack Newcastle Dark Ale Yeast, Lallemand CBC-1 (Cask and Bottle Conditioning), Lallemand Nottingham, Lallemand Windsor (British Ale), Fermentis Safale S-04, Fermentis US-05, Siebel Inst. American Ale BRY 96, Wyeast American Wheat 1010, Wyeast German Ale 1007, Wyeast K61sch 2565, Wyeast Kolsch II 2575-PC, White Labs Belgian Lager WLP815, White Labs Dusseldorf Alt WLP036, White Labs European Ale WLP011, White Labs German Ale/K61sch WLP029, East Coast Yeast K61schbier ECY21, Mangrove Jack Workhorse Beer Yeast, Siebel Inst. Alt Ale BRY 144, Wyeast American Ale 1056, Wyeast American Ale 111272, Wyeast British Ale 1098, Wyeast British Ale 111335, Wyeast Denny's Favorite 50 1450, Wyeast English Special Bitter 1768-PC, Wyeast Irish Ale 1084, Wyeast London Ale 1028, Wyeast London Ale III 1318, Wyeast London ESB Ale 1968, Wyeast Northwest Ale 1332, Wyeast Ringwood Ale 1187, Wyeast Thames Valley Ale 1275, Wyeast Thames Valley Ale II 1882-PC, Wyeast West Yorkshire Ale 1469, Wyeast Whitbread Ale 1099, White Labs American Ale Yeast Blend WLP060, White Labs Bedford British Ale WLP006, White Labs British Ale WLP005, White Labs Burton Ale WLPO23, White Labs California Ale V WLP051, White Labs California Ale WLP001, White Labs East Coast Ale WLP008, White Labs East Midlands Ale WLP039, White Labs English Ale WLP002, White Labs Essex Ale Yeast WLP022, White Labs Irish Ale WLP004, White Labs London Ale WLP013, White Labs Old Sonoma Ale WLP076, White Labs Pacific Ale WLPO41, White Labs Whitbread Ale WLP017, Coopers Pure Brewers' Yeast, Fermentis US-05, Muntons Premium Gold, Muntons Standard Yeast, Fermentis Safale S-04, Lallemand Nottingham, Lallemand Windsor (British Ale), Siebel Inst. American Ale BRY 96, White Labs American Hefeweizen Ale 320, White Labs Bavarian Weizen Ale 351, White Labs Belgian Wit Ale 400, White Labs Belgian Wit Ale II 410, White Labs Hefeweizen Ale 300, White Labs Hefeweizen IV Ale 380, Wyeast American Wheat 1010, Wyeast Bavarian Wheat 3638, Wyeast Bavarian Wheat Blend 3056, Wyeast Belgian Ardennes 3522, Wyeast Belgian Wheat 3942, Wyeast Belgian Witbier 3944, Wyeast Canadian/Belgian Ale 3864-PC, Wyeast Forbidden Fruit Yeast 3463, Wyeast German Wheat 3333, Wyeast Weihenstephan Weizen 3068, Siebel Institute Bavarian Weizen BRY 235, Fermentis Safbrew WB-06, Mangrove Jack Bavarian Wheat, Lallemand Munich (German Wheat Beer), Brewferm Blanche, Brewferm Lager, East Coast Yeast Belgian White ECY11, Augustiner, W-34/70, Andechs, D254, RC212, B0213. In some embodiments, the yeast is S. cerevisiae strain WLPOO1.

In some embodiments, the yeast strain for use with the genetically modified cells and methods described herein is a wine yeast strain. In some embodiments, the yeast strain for use with the genetically modified cells and methods described herein are red wine yeast strains (wine strains for production of red wines (e.g., Cabernet, Syrah, Pinot Noir, etc). Examples of yeast strains for use with the genetically modified cells and methods described herein include, without limitation, Red Star Montrachet, EC-1118, Elegance, Red Star Côte des Blancs, Epernay II, Red Star Premier Cuvee, Red Star Pasteur Red, Red Star Pasteur Champagne, Fermentis BCS-103, D254, RC212, B0213, and Fermentis VR44.

### Methods

Aspects of the present disclosure relate to methods of producing a fermented product using any of the genetically modified yeast cells described herein. Also provided are methods of producing ethanol using any of the genetically modified yeast cells described herein. In some embodiments, the method further involves adding a recombinant and/or purified enzyme to the fermentation, such as a glycosidase or an OMT enzyme, as described herein.

The process of fermentation exploits a natural process of using microorganisms to convert carbohydrates into alcohol and carbon dioxide. It is a metabolic process that produces chemical changes in organic substrates through enzymatic action. In the context of food production, fermentation broadly refers to any process in which the activity of microorganisms brings about a desirable change to a food product or beverage. The conditions for fermentation and the carrying out of a fermentation is referred to herein as a “fermentation process.”

In some aspects, the disclosure relates to a method of producing a fermented product, such as a fermented beverage, involving contacting any of the modified cells described herein with a medium comprising at least one fermentable sugar during a first fermentation process, to produce a fermented product. A “medium” as used herein, refers to liquid conducive to fermentation, meaning a liquid which does not inhibit or prevent the fermentation process. In some embodiments, the medium is water.

As also used herein, the term “fermentable sugar” refers to a carbohydrate that may be converted into an alcohol and carbon dioxide by a microorganism, such as any of the cells described herein. In some embodiments, the fermentable sugar is converted into an alcohol and carbon dioxide by an enzyme, such as a recombinant enzyme or a cell that expresses the enzyme. Examples of fermentable sugars include, without limitation, glucose, fructose, lactose, sucrose, maltose, and maltotriose.

In some embodiments, the fermentable sugar is provided in a sugar source. The sugar source for use in the claimed methods may depend, for example, on the type of fermented product and the fermentable sugar. Examples of sugar sources include, without limitation, wort, grains/cereals, fruit juice (e.g., grape juice and apple juice/cider), honey, cane sugar, rice, and koji. Examples of fruits from which fruit juice can be obtained include, without limitation, grapes, apples, blueberries, blackberries, raspberries, currants, strawberries, cherries, pears, peaches, nectarines, oranges, pineapples, mangoes, and passionfruit.

In some embodiments, the modified cells described herein are cultured in an anaerobic or semi-anaerobic environment. Anaerobic cell culture refers to the technique of culturing a microorganism, such as a modified yeast cell, in an environment without available oxygen. Semi-anaerobic cell culture refers to the technique of culturing a microorganism, such as a modified yeast cell, in an environment with limited oxygen availability, such as in a medium that has been pre-oxygenated.

As will be evident to one of ordinary skill in the art, in some instances, it may be necessary to process the sugar source in order to make available the fermentable sugar for fermentation. Using beer production as an example fermented beverage, grains (cereal, barley) are boiled or steeped in water, which hydrates the grain and activates the malt enzymes converting the starches to fermentable sugars, referred to as “mashing.” As used herein, the term “wort” refers to the liquid produced in the mashing process, which contains the fermentable sugars. The wort then is exposed to a fermenting organism (e.g., any of the cells described herein), which allows enzymes of the fermenting organism to convert the sugars in the wort to alcohol and carbon dioxide.

In some embodiments, the grains are malted, unmalted, or comprise a combination of malted and unmalted grains. Examples of grains for use in the methods described herein include, without limitation, barley, oats, maize, rice, rye, sorghum, wheat, karasumugi, and hatomugi.

In the example of producing sake, the sugar source is rice, which is incubated with koji mold (Aspergillus oryzae) converting the rice starch to fermentable sugar, producing koji. The koji then is exposed to a fermenting organism (e.g., any of the cells described herein), which allows enzymes of the fermenting organism to convert the sugars in the koji to alcohol and carbon dioxide.

In the example of producing wine, grapes are harvested, mashed (e.g., crushed) into a composition containing the skins, solids, juice, and seeds. The resulting composition is referred to as the “must.” The grape juice may be separated from the must and fermented, or the entirety of the must (i.e., with skins, seeds, solids) may be fermented. The grape juice or must is then exposed to a fermenting organism (e.g., any of the cells described herein), which allows enzymes of the fermenting organism to convert the sugars in the grape juice or must to alcohol and carbon dioxide.

In some embodiments, the methods described herein involve producing the medium, which may involve heating or steeping a sugar source, for example in water. In some embodiments, the water has a temperature of at least 50 degrees Celsius (50° C.) and is incubated with a sugar source for a period of time. In some embodiments, the water has a temperature of at least 75° C. and is incubated with a sugar source for a period of time. In some embodiments, the water has a temperature of at least 100° C. and is incubated with a sugar source for a period of time. Preferably, the medium is cooled prior to addition of any of the cells described herein.

In some embodiments, the methods described herein further comprise adding at least one (e.g., 1, 2, 3, 4, 5, or more) hop variety, for example to the medium, to a wort during a fermentation process. Hops are the flowers of the hops plant (Humulus lupulus) and are often used in fermentation to impart various flavors and aromas to the fermented product. Hops are considered to impart bitter flavoring in addition to floral, fruity, and/or citrus flavors and aromas and may be characterized based on the intended purpose. For example, bittering hops impart a level of bitterness to the fermented product due to the presence of alpha acids in the hop flowers, whereas aroma hops have lower lowers of alpha acids and contribute desirable aromas and flavor to the fermented product.

Whether one or more variety of hops is added to the medium and/or the wort and stage during which the hops are added may be based on various factors, such as the intended purpose of the hops. For example, hops that are intended to impart a bitterness to the fermented product are typically added during preparation of the wort, for example during boiling of the wort. In some embodiments, hops that are intended to impart a bitterness to the fermented product are added to the wort and boiled with the wort for a period of time, for example, for about 15-60 minutes. In contrast, hops that are intended to impart desired aromas to the fermented product are typically added later than hops used for bitterness. In some embodiments, hops that are intended to impart desired aromas to the fermented product are added to at the end of the boil or after the wort is boiled (i.e., “dry hopping”). In some embodiments, one or more varieties of hops may be added at multiple times (e.g., at least twice, at least three times, or more) during the method.

In some embodiments, the hops are added in the form of either wet or dried hops and may optionally be boiled with the wort. In some embodiments, the hops are in the form of dried hop pellets. In some embodiments, at least one variety of hops is added to the medium. In some embodiments, the hops are wet (i.e., undried). In some embodiments, the hops are dried, and optionally may be further processed prior to use. In some embodiments, the hops are added to the wort prior to the fermentation process. In some embodiments, the hops are boiled in the wort. In some embodiments, the hops are boiled with the wort and then cooled with the wort.

Many varieties of hops are known in the art and may be used in the methods described herein. Examples of hop varieties include, without limitation, Ahtanum, Amarillo, Apollo, Cascade, Centennial, Chinook, Citra, Cluster, Columbus, Crystal/Chrystal, Eroica, Galena, Glacier, Greenburg, Horizon, Liberty, Millennium, Mosaic, Mount Hood, Mount Rainier, Newport, Nugget, Palisade, Santiam, Simcoe, Sterling, Summit, Tomahawk, Ultra, Vanguard, Warrior, Willamette, Zeus, Admiral, Brewer's Gold, Bullion, Challenger, First Gold, Fuggles, Goldings, Herald, Northdown, Northern Brewer, Phoenix, Pilot, Pioneer, Progress, Target, Whitbread Golding Variety (WGV), Hallertau, Hersbrucker, Saaz, Tettnang, Spalt, Feux-Coeur Francais, Galaxy, Green Bullet, Motueka, Nelson Sauvin, Pacific Gem, Pacific Jade, Pacifica, Pride of Ringwood, Riwaka, Southern Cross, Lublin, Magnum, Perle, Polnischer Lublin, Saphir, Satus, Select, Strisselspalt, Styrian Goldings, Tardif de Bourgogne, Tradition, Bravo, Calypso, Chelan, Comet, El Dorado, San Juan Ruby Red, Sonnet Golding, Super Galena, Tillicum, Bramling Cross, Pilgrim, Hallertauer Herkules, Hallertauer Magnum, Hallertauer Taurus, Merkur, Opal, Smaragd, Halleratau Aroma, Kohatu, Rakau, Stella, Sticklebract, Summer Saaz, Super Alpha, Super Pride, Topaz, Wai-iti, Bor, Junga, Marynka, Premiant, Sladek, Styrian Atlas, Styrian Aurora, Styrian Bobek, Styrian Celeia, Sybilla Sorachi Ace, Hallertauer Mittelfrueh, Hallertauer Tradition, Tettnanger, Tahoma, Triple Pearl, Yakima Gold, and Michigan Copper.

In some embodiments, the fermentation process of at least one sugar source comprising at least one fermentable sugar may be carried out for about 1 day to about 31 days. In some embodiments, the fermentation process is performed for about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days or longer. In some embodiments, the fermentation process of the one or more fermentable sugars may be performed at a temperature of about 4° C. to about 30° C. In some embodiments, the fermentation process of one or more fermentable sugars may be carried out at temperature of about 8° C. to about 14° C. or about 18° C. to about 24° C. In some embodiments, the fermentation process of one or more fermentable sugars may be performed at a temperature of about 4° C., 5° C., 6° C., 7° C., 8° C., 9° C., 10° C., 11° C., 12° C., 13° C., 14C, 15° C., 16° C., 17° C., 18° C., 19° C., 20° C., 21° C., 22° C., 23° C., 24° C., 25° C., 26° C., 27° C., 28° C., 29° C., or 30° C.

In some embodiments, fermentation results in the reduction of the amount of fermentable sugar present in a medium. In some embodiments, the reduction in the amount of fermentable sugar occurs within 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or longer, from the start of fermentation. In some embodiments, the amount of fermentable sugar is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, at least 99.6%, at least 99.7%, at least 99.8%, at least 99.9%, or 100%. In some embodiments, the modified cell or cells ferment a comparable or greater amount of fermentable sugar, relative to the amount of fermentable sugar fermented by wild-type yeast cells in the same amount of time.

The methods described herein may involve at least one additional fermentation process. Such additional fermentation methods may be referred to as secondary fermentation processes (also referred to as “aging” or “maturing”). As will be understood by one of ordinary skill in the art, secondary fermentation typically involves transferring a fermented beverage to a second receptacle (e.g., glass carboy, barrel) where the fermented beverage is incubated for a period of time. In some embodiments, the secondary fermentation is performed for a period of time between 10 minutes and 12 months. In some embodiments, the secondary fermentation is performed for 10 minutes, 20 minutes, 40 minutes, 40 minutes, 50 minutes, 60 minutes (1 hour), 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 3 months, 4 months, 5 months, 6 months, 7 months, 8 months, 9 months, 10 months, 11 months, 12 months, or longer. In some embodiments, the additional or secondary fermentation process of the one or more fermentable sugars may be performed at a temperature of about 4° C. to about 30° C. In some embodiments, the additional or secondary fermentation process of one or more fermentable sugars may be carried out at temperature of about 8° C. to about 14° C. or about 18° C. to about 24° C. In some embodiments, the additional or secondary fermentation process of one or more fermentable sugars may be performed at a temperature of about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C.

As will be evident to one of ordinary skill in the art, selection of a time period and temperature for an additional or secondary fermentation process will depend on factors such as the type of beer, the characteristics of the beer desired, and the yeast strain used in the methods.

In some embodiments, one or more additional flavor components may be added to the medium prior to or after the fermentation process. Examples include, hop oil, hop aromatics, hop extracts, hop bitters, and isomerized hops extract.

Products from the fermentation process may volatilize and dissipate during the fermentation process or from the fermented product. For example, volatile phenols produced during fermentation using the cells described herein may dissipate or evaporate resulting in reduced levels of volatile phenols in the fermented product.

Any of the methods described herein may involve removing one or more volatile phenols from the fermented product. In some embodiments, removing or reducing the level of volatile phenols in the fermented product involves subjecting the fermented product to one or more additional processes, such as filtering (e.g., reverse osmosis), contacting the fermented product with a fining agent, or modifying the volatile phenols (e.g., chemical modification such as methylation).

In some embodiments, the methods involve subjecting the fermented product to a filtration process. Filtration methods suitable for removal of volatile phenols from a fermented product are known in the art. In some embodiments, the filtration process is reverse osmosis, which involves passing the fermented product through a membrane (filter) having a molecular weight cut-off sufficient to remove volatile phenols from the fermented product.

In some embodiments, the methods involve contacting the fermented product with a fining agent. Examples of fining agents for removal of smoke taint include activated carbon and cyclodextrin polymers. Additional example processes and fining agents for removing volatile phenols are known in the art. See, e.g., Mirabelli-Montan et al. Molecules (2021) 26: 1672.

In some embodiments, removing or reducing the level of volatile phenols in the fermented product involves subjecting the fermented product to an enzymatic process to modify the volatile phenol, for example contacting the fermented product with an enzyme capable of removing the undesired phenol or converting the undesired volatile phenol into a neutral or more desirable form. In some embodiments, removing or reducing the level of volatile phenols in the fermented product involves contacting the fermented product with an enzyme having O-methyltransferase activity, such as any of the enzymes having O-methyltransferase activity described herein. In some embodiments, the enzyme having O-methyltransferase activity is a purified or isolated recombinant enzyme. In some embodiments, the enzyme having O-methyltransferase activity is expressed by a genetically modified yeast cell, which may be the same genetically modified cell that expresses the heterologous gene encoding an enzyme having glycosidase activity or a second genetically modified cell. In some embodiments, removing or reducing the level of volatile phenols in the fermented product involves contacting the fermented product with a genetically modified cell that expresses the enzymes having O-methyltransferase activity. In some embodiments, the fermented product is first contacted with a genetically modified cell that expresses an enzyme having O-methyltransferase activity, then the fermented product is contacted with a purified or isolated recombinant enzyme having O-methyltransferase activity. In some embodiments, the fermented product is first contacted with a genetically modified cell that expresses an enzyme having glycosidase activity, then the fermented product is contacted with a purified or isolated recombinant enzyme having OMT activity. In some embodiments, the fermented product is first contacted with a purified or isolated recombinant enzyme having glycosidase activity and then the fermented product is contacted with a genetically modified cell that expresses an enzyme having OMT activity.

Various refinement, filtration, and aging processes may occur subsequent fermentation, after which the liquid is bottled (e.g., captured and sealed in a container for distribution, storage, or consumption). Any of the methods described herein may further involve distilling, pasteurizing, and/or carbonating the fermented product. In some embodiments, the methods involve carbonating the fermented product. Methods of carbonating fermented beverages are known in the art and include, for example, force carbonating with a gas (e.g., carbon dioxide, nitrogen), naturally carbonating by adding a further sugar source to the fermented beverage to promote further fermentation and production of carbon dioxide (e.g., bottle conditioning).

### Fermented Products

Aspects of the present disclosure relate to fermented products produced by any of the methods disclosed herein. In some embodiments, the fermented product is a fermented beverage. Examples of fermented beverages include, without limitation, beer, wine, sake, mead, cider, cava, sparkling wine (champagne), kombucha, ginger beer, water kefir. In some embodiments, the beverage is beer. In some embodiments, the beverage is wine. In some embodiments, the beverage is sparkling wine. In some embodiments, the beverage is Champagne. In some embodiments, the beverage is sake. In some embodiments, the beverage is mead. In some embodiments, the beverage is cider. In some embodiments, the beverage is hard seltzer. In some embodiments, the beverage is a wine cooler.

In some embodiments, the fermented product is a fermented food product. Examples of fermented food products include, without limitation, cultured yogurt, tempeh, miso, kimchi, sauerkraut, fermented sausage, bread, and soy sauce.

Aspects of the present disclosure relate to reducing the production of undesired products such as volatile phenols, during fermentation of a product. In some embodiments, expression of the glycosidase and/or O-methyltransferase in the genetically modified cells described herein result in a reduction in the production of an undesired product by about 5%, 10%, 15%, 20%, 25%, 30%, 35%, 40%, 45%, 50%, 55%, 60%, 65%, 70%, 75%, 80%, 85%, 90%, 95% or more relative to production of the undesired product (e.g., volatile phenols) by use of a wild-type yeast cell or a yeast cell that does not express the enzyme(s).

Aspects of the present disclosure relate to reducing the presence of undesired products such as volatile phenols, in a fermented product. In some embodiments, expression of the glycosidase and/or O-methyltransferase in the genetically modified cells described herein result in a reduction of an undesired product in a fermented product by about 5%, 10%, 15%, 20%, 25%, 30%, 35%, 40%, 45%, 50%, 55%, 60%, 65%, 70%, 75%, 80%, 85%, 90%, 95% or more relative to the level of the undesired product (e.g., volatile phenols) in a fermented product produced using a wild-type yeast cell or a yeast cell that does not express the enzyme(s).

In some embodiments, the methods described herein result in a reduced level of one or more volatile phenol in the fermented product. Non-limiting examples of volatile phenols include, without limitation, guaiacol, m-cresol, p-cresol, o-cresol, phenol, 4-methylguaiacol, syringol, and/or 4-methylsyringol.

As described herein, the production of volatile phenols can impart a smoke-like aroma to fermented products. In some embodiments, the titer of volatile phenols is less than 100 μg L⁻¹, for example less than 100, 95, 90, 85, 80, 75, 70, 65, 60, 55, 50, 45, 40, 35, 30, 25, 20, 15, 10, 9.5, 9.0, 8.5, 8.0, 7.5, 7.0, 6.5, 6.0, 5.5, 5.0, 4.5, 4.0, 3.5, 3.0, 2.5, 2.0, 1.5, 1.0, 0.9, 0.8, 0.7, 0.6, 0.5, 0.4, 0.3, 0.2, 0.1 μg L⁻¹ or less. In some embodiments, the titer of volatile phenols is reduced in a fermented product by about 5%, 10%, 15%, 20%, 25%, 30%, 35%, 40%, 45%, 50%, 55%, 60%, 65%, 70%, 75%, 80%, 85%, 90%, 95% or more relative to the level of the undesired product (e.g., volatile phenols) in a fermented product produced using a wild-type yeast cell or a yeast cell that does not express the enzyme(s) (e.g., glycosidase and/or O-methyltransferase). In some embodiments, the titer of the volatile phenol is below the limit of human detection.

Methods of measuring titers/levels of volatile phenols will be evident to one of ordinary skill in the art. In some embodiments, the titers/levels of non-volatile phenolic glycosides and/or volatile phenols are measured using gas-chromatography mass-spectrometry (GC/MS). In some embodiments, the titers/levels of non-volatile phenolic glycosides and/or volatile phenols are measured using liquid-chromatography mass-spectrometry (LC/MS). In some embodiments, the titers/levels of non-volatile phenolic glycosides and/or volatile phenols are assessed using sensory panels, including for example human taste-testers.

In some embodiments, the fermented beverage contains an alcohol by volume (also referred to as “ABV,” “abv,” or “alc/vol”) between 0.1% and 30%. In some embodiments, the fermented beverage contains an alcohol by volume of about 0.1%, 0.2%, 0.3%, 0.4%, 0.5%, 0.6%, 0.07%, 0.8%, 0.9%, 1.0%, 1.1%, 1.2%, 1.3%, 1.4%, 1.5%, 1.6%, 1.7%, 1.8%, 1.9%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or higher. In some embodiments, the fermented beverage is non-alcoholic (e.g., has an alcohol by volume less than 0.5%).

### Kits

Aspects of the present disclosure also provide kits for use of the genetically modified yeast cells, for example to produce a fermented beverage, fermented product, or ethanol. In some embodiments, the kit contains a modified cell containing a heterologous gene encoding an enzyme with glycosidase activity and/or a heterologous gene encoding an enzyme with O-methyltransferase activity.

In some embodiments, the kit is for the production of a fermented beverage. In some embodiments, the kit is for the production of beer. In some embodiments, the kit is for the production of wine. In some embodiments, the kit is for the production of sake. In some embodiments, the kit is for the production of mead. In some embodiments, the kit is for the production of cider.

The kits may also comprise other components for use in any of the methods described herein, or for use of any of the cells as described herein. For example, in some embodiments, the kits may contain grains, water, wort, must, yeast, hops, juice, or other sugar source(s). In some embodiments, the kit may contain one or more fermentable sugars. In some embodiments, the kit may contain one or more additional agents, ingredients, or components.

Instructions for performing the methods described herein may also be included in the kits described herein.

The kits may be organized to indicate a single-use compositions containing any of the modified cells described herein. For example, the single use compositions (e.g., amount to be used) can be packaged compositions (e.g., modified cells) such as packeted (i.e., contained in a packet) powders, vials, ampoules, culture tube, tablets, caplets, capsules, or sachets containing liquids.

The compositions (e.g., modified cells) may be provided in dried, lyophilized, frozen, or liquid forms. In some embodiments, the modified cells are provided as colonies on an agar medium. In some embodiments, the modified cells are provided in the form of a starter culture that may be pitched directly into a medium. When reagents or components are provided as a dried form, reconstitution generally is by the addition of a solvent, such as a medium. The solvent may be provided in another packaging means and may be selected by one skilled in the art.

A number of packages or kits are known to those skilled in the art for dispensing a composition (e.g., modified cells). In certain embodiments, the package is a labeled blister package, dial dispenser package, tube, packet, drum, or bottle.

Any of the kits described herein may further comprise one or more vessel for performing the methods described herein, such as a carboy or barrel.

### General Techniques

The practice of the subject matter of the disclosure will employ, unless otherwise indicated, conventional techniques of molecular biology (including recombinant techniques), microbiology, cell biology, biochemistry, and immunology, which are within the skill of the art. Such techniques are explained fully in the literature, such as, but without limiting, Molecular Cloning: A Laboratory Manual, J. Sambrook, et al., eds., Fourth Edition, Cold Spring Harbor Laboratory Press, Cold Spring Harbor, New York, 2012; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999).

### Equivalents and Scope

It is to be understood that this disclosure is not limited to any or all of the particular embodiments described expressly herein, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this disclosure are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc.) the pronoun shall be construed as gender neutral (i.e., construed to refer to all genders equally) regardless of the implied gender unless the context clearly indicates or requires otherwise. Wherever used herein, words used in the singular include the plural, and words used in the plural include the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

### EXAMPLES

### Example 1

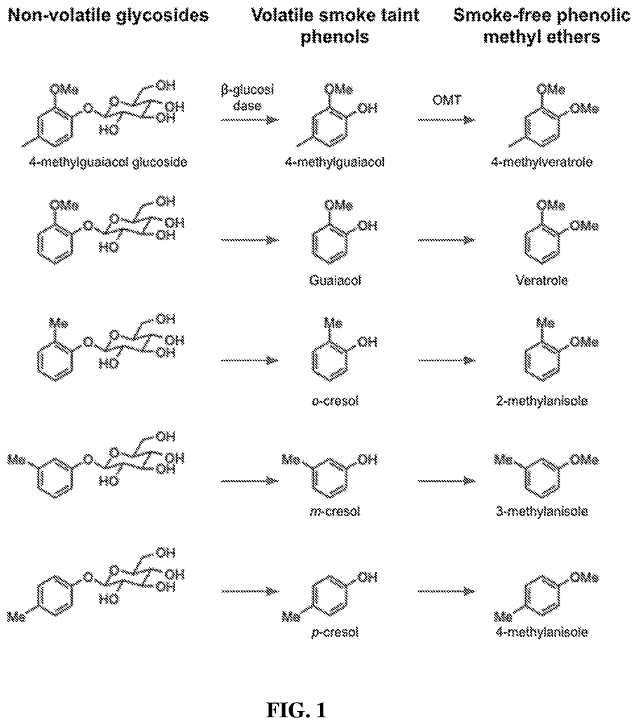

Expression of glycosidase enzymes to release volatile phenols from sugar conjugates Grapes and hops exposed to smoke absorb volatile phenols produced by burning wood and metabolize them into non-volatile phenolic glycosides. Over time, volatile phenols are released from the non-volatile phenolic glycosides, creating smoke-taint off-flavors in the resultant fermented product (FIG. 1). The present disclosure relates to methods and compositions for removing volatile phenols before, during, or after fermentation, in order to reduce or eliminate smoke-taint in fermented products that contain grapes and/or hops that have been exposed to smoke.

In effort to release free, volatile phenols from their non-volatile phenolic glycoside precursors, genetically engineered S. cerevisiae strains are constructed to express glycosidase enzymes that hydrolyze glycosidic bonds. The glycosidases may be secreted to enable hydrolysis of glycosides outside of the cell, since transport into the cell is likely to be limited. Glycosidases occur widely in nature and each has distinct substrate specificities and catalytic activities. S. cerevisiae encodes multiple glycosidases, but few are known to be secreted and none have been characterized for activity on smoke taint substrates.

Prior efforts have been made to engineer glycosidase expression in yeast ((Kaya, et al., Appl. Microbiol. Biotechnol. (2008) 79: 51-60), (Adam, et al., Yeast. (1995) 11: 395-406), (Sinchez-Torres, et al., J. Agric. Food Chem. (1998) 46: 354-360), (Larue, et al., Biotechnol. Biofuels. (2016) 9: 52)). Manzanares et al. engineered a wine yeast to secrete the Aspergillus aculeatus rhaA gene, encoding an alpha-L-rhamnosidase (Manzanares, et al., Appl. Environ. Microbiol. (2003) 69: 7558-7562). They engineered a second strain to express the Candida molischiana gene bgln, which encodes a beta-D-glucosidase. Microvinification trials carried out with these strains showed an increase in monoterpene glycoside hydrolysis and free monoterpene content in wine. Ishikawa et al. expressed the Aspergillus oryzae RhaA gene, encoding an alpha-L-rhamnosidase, in Pichia pastoris (Ishikawa, et al., J. Biosci. Bioeng. (2017) 124: 630-634). When fused with the S. cerevisiae α-factor secretion signal peptide, the enzyme was secreted into the supernatant. The secreted enzyme was then purified and characterized. Other groups have engineered beta-glucosidase expression in yeast for other applications (isoflavone production (Kaya, et al., Appl. Microbiol. Biotechnol. (2008) 79: 51-60), cellobiose utilization ((Eriksen, et al., Microb. Cell Fact. (2013) 12: 61), (Hu, et al., Front. Microbiol. (2016) 7: 241), (Galazka, et al., Science. (2010 330: 84-86)), cellulose degradation (Oh, et al., FEMS Yeast Res. (2020) 20)). However, none of this work has been done in the context of smoke taint mitigation.

To identify glycosidase enzymes with high activity on phenolic glycoside substrates, glycosidase candidates are expressed and screened in yeast cells, such as glucosidases (which remove glucose) and rhamnosidases (which remove rhamnose). The glycosidases are tested in combination with several secretion signal peptides (e.g., SED, MAT, MAT pre-sequence, TFP5-1, TFP1-4, TFP10, TFP23, SUC2, SRLU, and KSH1) to identify the glycosidase-secretion signal pair that displays the greatest glycoside hydrolysis activity for expression in S. cerevisiae wine and brewing strains. Table 1 shows exemplary genes encoding the glycosidase enzymes that will be screened for glycoside hydrolysis activity.

TABLE 1
Genes encoding glycosidase enzymes

| SEQ | | | | |
|---|---|---|---|---|
| ID # | Gene | Accession number | Source Organism | Activity |
| 1 | RhaB | AF284762 | Aspergillus aculeatus | alpha-L- |
| | | | | rhamnosidase |
| 2 | RhaA | AF284761 | Aspergillus aculeatus | alpha-L- |
| | | | | rhamnosidase |
| 3 | AtRha | AFH54529 | Aspergillus terreus | alpha-L- |
| | | | | rhamnosidase |
| 4 | AndRha | CBF86381.1 | Aspergillus nidulans | alpha-L- |
| | | | | rhamnosidase |
| 5 | AndRha | FR873475 | Aspergillus nidulans | alpha-L- |
| | | | | rhamnosidase |
| 6 | AngRha | XP_001389086.1 | Aspergillus niger | alpha-L- |
| | | | | rhamnosidase |
| 7 | AorhaA | BAE58354.1 | Aspergillus oryzae | alpha-L- |
| | | | | rhamnosidase |
| 8 | RhaB1 | FJ943501 | Lactobacillus pantarum | alpha-L- |
| | | | NCC245 | rhamnosidase |
| 9 | RhaB2 | FJ943501 | Lactobacillus pantarum | alpha-L- |
| | | | NCC245 | rhamnosidase |
| 10 | RhmA | AAR96046 | Thermomicrobia PRI- | alpha-L- |
| | | | 1686 | rhamnosidase |
| 11 | RhmB | AAR96047 | Thermomicrobia PRI- | alpha-L- |
| | | | 1686 | rhamnosidase |
| 12 | bgln | U16259.1 | Candida molischiana | beta-D-glucosidase |
| 13 | OepGLU | KX278417 | Olea europaea | beta-glucosidase |
| 14 | EGH1 | NP_012272.3 | Saccharomyces cerevisiae | beta-glucosidase |
| 15 | Tabgl1 | DQ114397 | Thermoascus aurantiacus | beta-glucosidase |
| | | | IFO9748 | |
| 16 | AoBGL1 | Q2UIR4 | Aspergillus oryzae | beta-glucosidase |
| 17 | AnBGL1 | AspGD An18g03570 | Aspergillus niger | beta-glucosidase |
| 18 | TrBGL2 | AB003110 | Trichoderma reesei | beta-glucosidase |
| 19 | TrBGL1 | U09580 | Trichoderma reesei | beta-glucosidase |
| 20 | BGL1 | EAA26868.1 | Neurospora crassa | beta-glucosidase |
| 21 | BGL2 | EAA30164.1 | Neurospora crassa | beta-glucosidase |
| 22 | bglH | BAE64214.1 | Aspergillus oryzae | beta-glucosidase |
| 23 | OoBgl | EAV39986.1 | Oenococcus oeni | beta-glucosidase |
| 24 | OoBgl | WP_011677766.1 | Oenococcus oeni | beta-glucosidase |
| 25 | Ph691 | H9ZGE0 | Prunus dulcis | beta-glucosidase |
| 26 | Ph692 | H9ZGE2 | Prunus dulcis | beta-glucosidase |
| 27 | BsBglA | Q03506 | Bacillus circulans | beta-glucosidase |
| 28 | SmBgl | F7X9K8 | Sinorhizobium meliloti | beta-glucosidase |

Once free, volatile phenols can be removed using a filtration process (e.g., reverse osmosis), contacting with a fining agent, or modified further (e.g., by enzymatic modification).

### Expression of O-Methyltransferases to Convert Phenol Hydroxyl Functional Group to a Methoxy Functional Group

Following release of free, volatile phenols by hydrolysis of non-volatile phenolic glycoside, free, volatile phenols may be chemically modified to no longer contribute smoke-related sensory notes to fermented products. Modification of the hydroxyl groups of the phenolic compounds can alter their sensory contribution O-methyltransferase (OMT) enzymes are evaluated for the ability to catalyze phenol methylation in a glycosidase overexpressing strain.

Methylation of volatile phenols may vastly improve the aroma profile of a smoke-tainted fermented product. OMTs from a variety of plant species will be screened to identify OMTs that possess high activity for methylation of guaiacol and m-cresol. Table 2 shows genes encoding OMT enzymes to be evaluated for guaiacol and m-cresol methylation activity.

TABLE 2
Genes encoding OMT enzymes

| SEQ ID # | Gene | Accession number | Source organism |
|---|---|---|---|
| 37 | SIGOMT1 | (have protein sequence) | Silene latifolia (white campion) |
| 38 | SIOMT1 | MW380256 | Solanum lycopersicum (tomato) |
| 39 | SIOMT4 | MW380257 | Solanum lycopersicum (tomato) |
| 40 | SICTOMT1 | Sol Genomics Network | Solanum lycopersicum (tomato) |
| | | SGN-U582403 | |
| 41 | RhOOMT1 | AF502433.1 | Rosa hybrida (rose) |
| 42 | RhOOMT2 | AF502434 | Rosa hybrida (rose) |
| 43 | EOMT1 | AF435008 | Ocimum basilicum (basil) |
| 44 | EjOMT1 | LC127201 | Eriobotrya japonica (loquat) |
| 45 | AEOMT | U39301 | Pinus taeda (loblolly pine) |
| 46 | FaOMT | AF220491.2 | Fragaria ananassa (strawberry) |
| 47 | SpCOMT | NP_595284.1 | Schizosaccharomyces pombe |
| 48 | COMT1 | AAD38189 | Ocimum basilicum (basil) |
| 49 | ZeCAOMT | U19911 | Zinnia elegans (zinnia |

### Example 2

### O-Methyltransferase Activity Screen on 5 Major Smoke Taint Phenols

Yeast strains (S. cerevisiae, strain CEN.PK2-1D) were transformed with a screening plasmid encoding an O-methyltransferase (OMT) enzyme from one of five organisms (white campion, tomato, rose, basil, and loquat), driven by the yeast GAL1 promoter. 24 hrs after induction with galactose, 10 mL yeast cultures were pelleted by centrifugation and then suspended in 1 mL phosphate buffered saline (PBS) at pH 7.4. Yeast cells were lysed by mechanical disruption aided by glass beads, and the resulting lysate was clarified by centrifugation. The total protein content was normalized upon concentration estimation by absorbance measurement at 280 nm.

To measure the activities of the OMT enzymes on smoke taint phenols, each 500 μL in vitro reaction in a 96-well deep well plate was charged with PBS at pH 7.4, 50 μL yeast lysate, 2 mM S-adenosylmethionine, and 1 mM smoke taint phenol (4-methylguaiacol, guaiacol, o-cresol, m-cresol, or p-cresol). The reactions were carried out at room temperature for 2 hrs, then extracted with ethyl acetate.

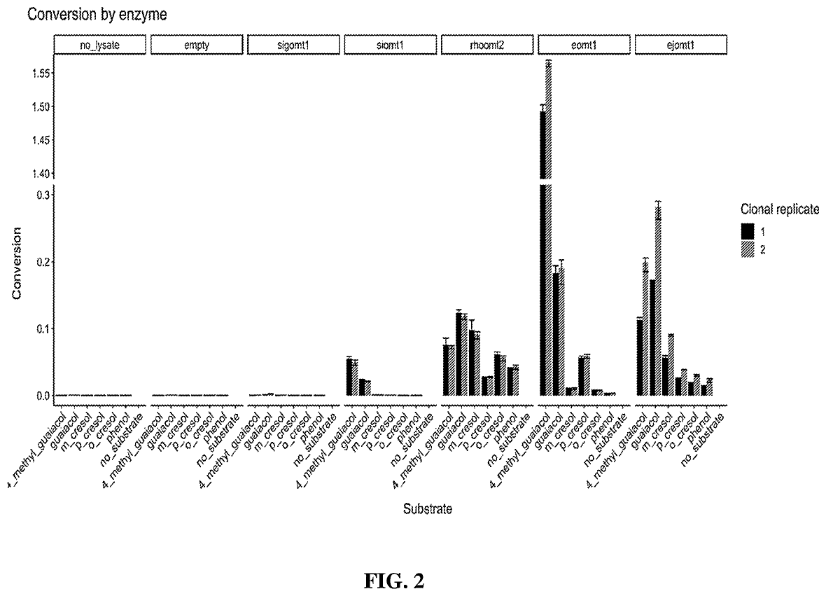

Ethyl acetate extracts were analyzed by gas chromatography-mass spectrometry (GC-MS) using a linear temperature ramp from 60° C. to 150° C. over 10 min. The retention time and fragmentation of each phenol substrate and methyl ether product was verified by authentic standards. Enzyme activity was qualitatively compared in this experiment by calculating the conversion ratio of each reaction, defined as the peak area of the methyl ether product divided by the peak area of the phenolic substrate. Measurable conversion of all five smoke taint phenols by three out of the five enzymes was observed (rose, basil, and loquat) (FIG. 2). These data demonstrate that each of the rose, basil, and loquat OMT enzymes were able to convert smoke taint phenols into methylated volatile phenols, thereby removing the smoke taint sensory notes.

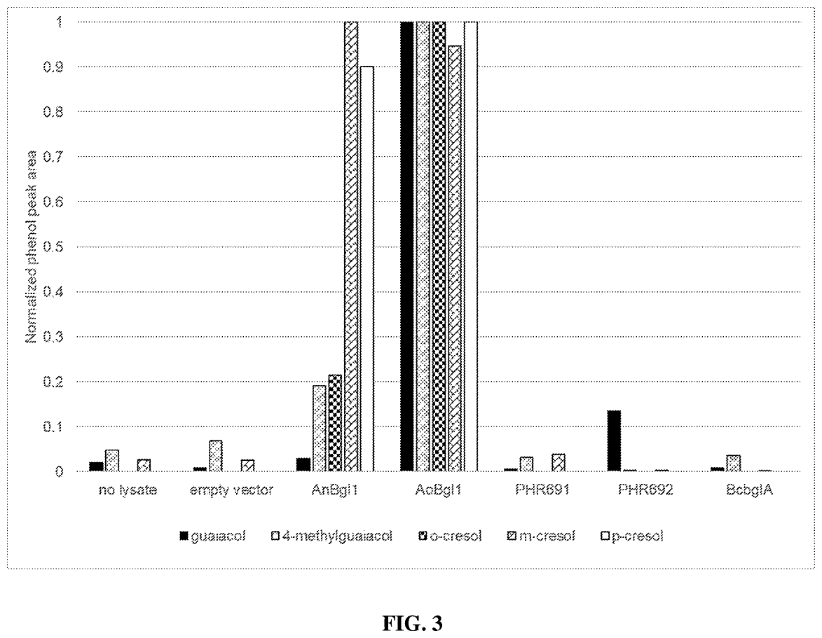

β-Glucosidase Activity Screen on 5 Major Smoke Taint Monoglucosides Yeast strains (S. cerevisiae, strain CEN.PK2-1D) were transformed with a screening plasmid containing a β-glucosidase driven by the yeast GAL1 promoter. In total, five 3-glucosidase enzymes were screened for activity against smoke taint monoglucoside substrates. Lysates of the yeast strains containing active β-glucosidase enzyme were then prepared after 48 hours of aerobic protein expression induction in galactose-containing media. The lysates were combined with 0.1 mg/mL monoglucoside substrate in aqueous buffer at pH 3.8 to mimic the acidic environment of wine/grape juice. Enzymatic conversion of monoglucosides to phenols was allowed to proceed for 9 days at room temperature without agitation, to mimic the conditions of wine fermentation. The lysate mixture containing both monoglucoside reactant and phenolic product was then diluted into 5% acetonitrile and analyzed by LC/MS. These data demonstrate that both glucosidases, AoBgl1 and AnBgl1, were able to convert non-volatile phenolic glucosides (phenol bound to glucose) to volatile phenols (FIG. 3).
Secretion Signal Peptide Screen with AoBgl1 β-Glucosidase

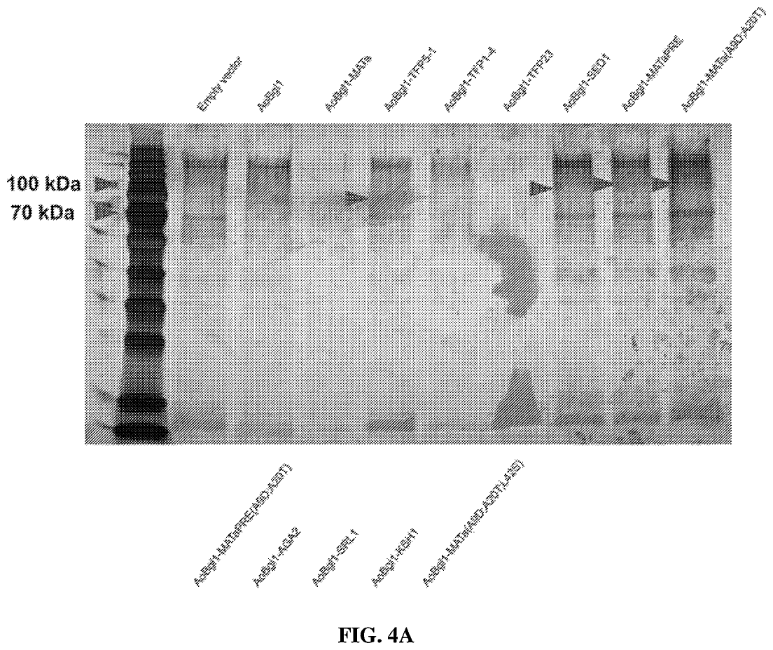

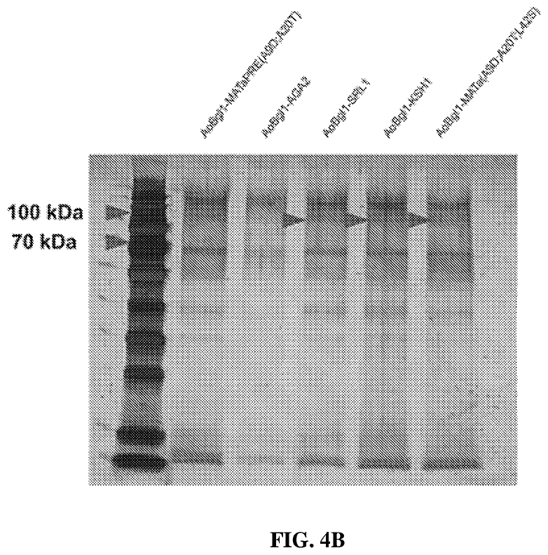

Yeast strains (S. cerevisiae, strain CEN.PK2-1D) were transformed with a screening plasmid containing β-glucosidase from Aspergillus oryzae (AoBgl1) with a 5′ secretion signal peptide driven by the yeast GAL1 promoter. In total, twelve secretion signal peptides were screened for their ability to promote transport of AoBgl1 into the extracellular space (FIGS. 4A-4B). Secreted proteins were concentrated from culture supernatant after 48 hours of aerobic protein expression induction in galactose-containing media. Concentrated secreted proteins were separated by polyacrylamide gel electrophoresis and visualized by silver staining. The expected molecular weight of secreted β-glucosidase is between 90-100 kDa, depending on the identity of the secretion signal peptide. These data demonstrate that genetically modified yeast cells are able to secrete AoBgl1 when the enzyme is fused to secretion peptides TFP5-1, SED1, MATαPRE, MATα(A9D;A20T), SRL1, KSH1, or MATα(A9D;A20T;L42S).

### AoBgl1 Activity on 5 Major Smoke Taint Monoglucosides at Product-Relevant Concentrations

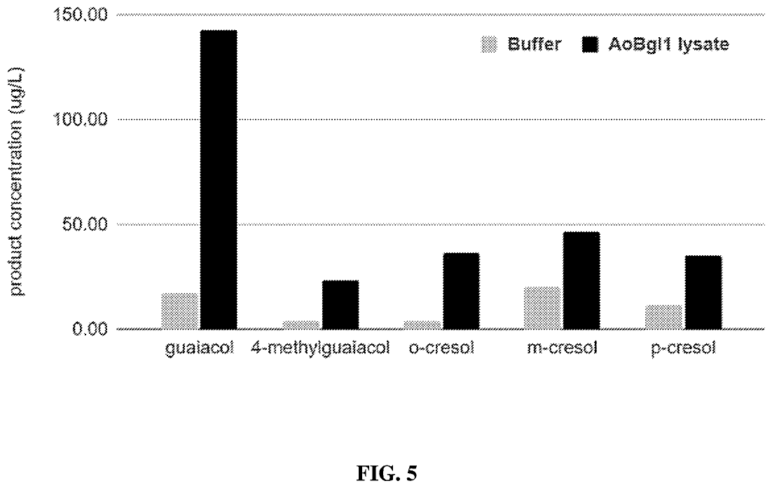

Yeast strains (S. cerevisiae, strain CEN.PK2-1D) were transformed with a screening plasmid containing β-glucosidase from Aspergillus oryzae (AoBgl1) driven by the yeast GAL1 promoter. Lysates from yeast strains containing active AoBgl1 enzyme was then prepared after 48 hours of aerobic protein expression induction in galactose-containing media. The lysates were combined with 500 μg/L monoglucoside substrate in aqueous buffer at pH 3.8 to mimic the acidic environment of wine/grape juice. 500 μg/L is close to reported concentrations of smoke taint glucosides in tainted grapes or juice. Enzymatic conversion of monoglucosides to phenols was allowed to proceed for 9 days at room temperature without agitation, to mimic the conditions of wine fermentation. A buffer-only control was included to account for spontaneous hydrolysis of the glucoside bond. Phenolic products were then separated from the aqueous lysate matrix by organic extraction, analyzed by GC/MS, and quantified with authentic standards (FIG. 5). These data demonstrate that AoBgl1 converts each of the five non-volatile phenolic glucosides to the corresponding volatile phenols at fermentation product-relevant concentrations (500 μg/L).

### Purified β-Glucosidase Activity on 5 Major Smoke Taint Monoglucosides at Fermentation Product-Relevant Concentrations

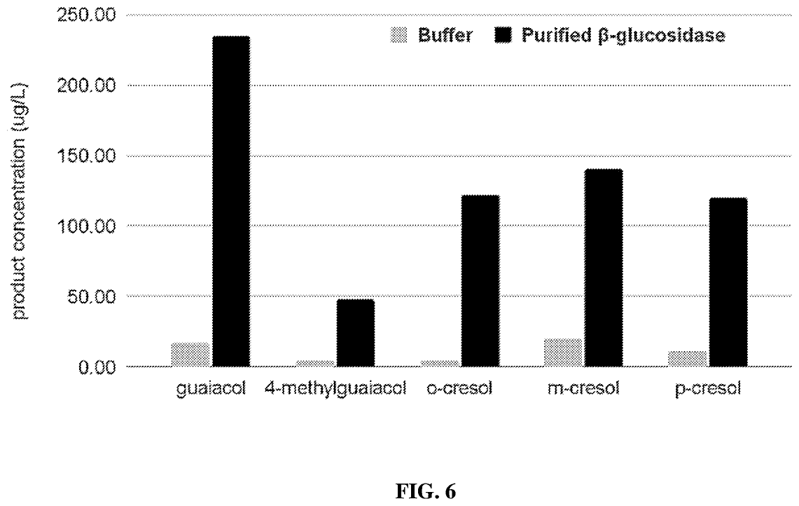

Recombinant β-glucosidase from almonds was purchased as a pure, lyophilized powder and reconstituted in aqueous solution. The enzyme solution was diluted with 500 g/L monoglucoside substrate in an aqueous buffer at pH 3.8 to mimic the acidic environment of wine/grape juice. 500 μg/L is close to reported concentrations of smoke taint glucosides in tainted grapes or juice. Enzymatic conversion of monoglucosides to phenols was allowed to proceed for 9 days at room temperature without agitation, to mimic the conditions of wine fermentation. A buffer-only control was included to account for spontaneous hydrolysis of the glucoside bond. Phenolic products were then separated from the aqueous lysate matrix by organic extraction, analyzed by GC/MS, and quantified with authentic standards (FIG. 6). These data demonstrate that the added purified β-glucosidase is able to convert non-volatile phenolic glucoside to volatile phenols.

### Example 3

Small-Scale Wine Fermentations with Strains Expressing Selected O-Methyltransferases

A wine yeast strain referred to as D254, commonly used to make red wines such as Syrah and Pino Noir, was engineered to constitutively express an O-methyltransferase enzyme derived from basil (EOMT1 from Ocimum basilicum) or loquat (Eriobotrya japonica, EjOMT1), corresponding to engineered strains y1375 and y1376, respectively. Engineered strains y1375 and y1376, along with the parent strain D254, were inoculated into Pinot Noir grape juice containing 500 μg/L of volatile phenol substrate. Anaerobic wine fermentation was allowed to proceed for five days at 32° C. Volatile phenol substrates and phenolic methyl ether products were then extracted from the extracellular wine matrix, analyzed by GC/MS, and quantified with authentic standards.

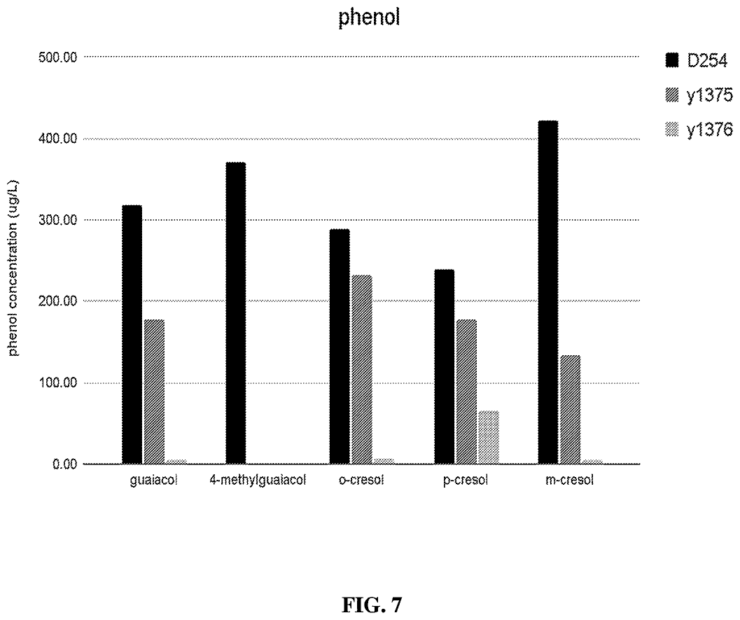

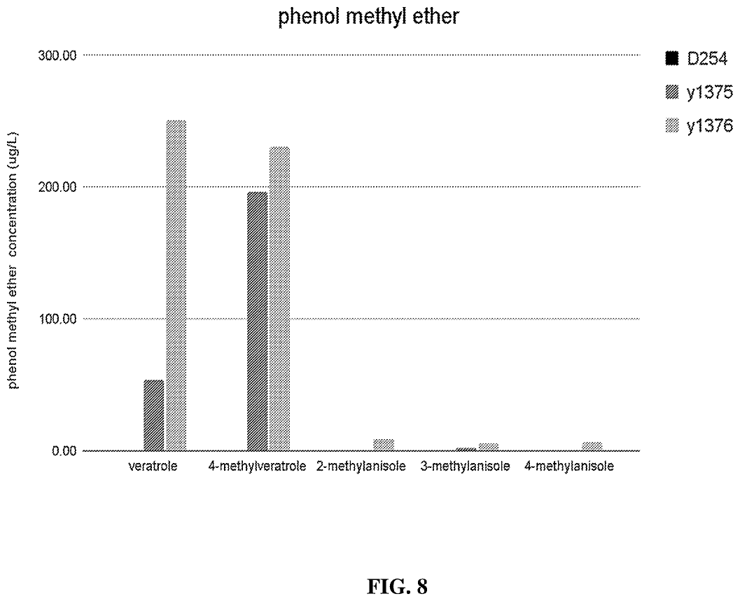

Based on these data, during wine fermentation, genetically modified yeast cells expressing an O-methyltransferase (Table 3) were able to consume volatile smoke taint phenols. Specifically, engineered yeast strains y1375 (expressing EjOMT1 from basil) and y1376 (expressing EOMT1 from loquat) converted the volatile phenols guaiacol and 4-methylguaicol to the phenolic methyl ethers veratrole and 4-methylveratrole, respectively (FIG. 7 and FIG. 8). In addition, engineered yeast strain y1376 converted volatile phenols o-cresol, p-cresol, and m-cresol partially to the phenolic methyl ethers 2-methylanisole, 3-methylanisole, and 4-methylanisole, respectively, and partially to an unknown side product (FIG. 7 and FIG. 8).

TABLE 3
Strains used in Example 3

| | | | Strain |
|---|---|---|---|
| Name | Genotype | Description of Genes | Background |
| y1375 | PDC6::pTDH3-EOMT1 | Basil OMT | D254 |
| y1376 | PDC6::pTDH3-EjOMT1 | Loquat OMT | D254 |
| y1384 | FIG2::pTDH3- | Aspergillus oryzae β- | D254 |
| | AoBgl1(N: SED1) | glucosidase | |
| | | with N-terminal SED1 | |
| | | secretion signal | |
| y1385 | FIG2::pTDH3- | A. oryzae β-glucosidase with | D254 |
| | AoBgl1(N: MATα(A9D; A20T) | N-terminal MATα(A9D; A20T) | |
| | | secretion signal | |
| y1386 | PDC6::pTDH3-EOMT1, | Basil OMT, A. oryzae | D254 |
| | FIG2::pTDH3- | β-glucosidase with N-terminal | |
| | AoBgl1(N: TFP5-1) | TFP5-1 secretion signal | |
| y1387 | PDC6::pTDH3-EOMT1, | Basil OMT, A. oryzae | D254 |
| | FIG2::pTDH3- | β-glucosidase with N-terminal | |
| | AoBgl1(N: SED1) | SED1 secretion signal | |
| y1388 | PDC6::pTDH3-EjOMT1, | Loquat OMT, A. oryzae | D254 |
| | FIG2::pTDH3- | β-glucosidase with N-terminal | |
| | AoBgl1(N: TFP5-1) | TFP5-1 secretion signal | |
| y1389 | PDC6::pTDH3-EjOMT1, | Loquat OMT, A. oryzae | D254 |
| | FIG2::pTDH3- | β-glucosidase with N-terminal | |
| | AoBgl1(N: SED1) | SED1 secretion signal | |

Small Scale Wine Fermentations with Spiked-In β-Glucosidase and Strains Expressing OMT

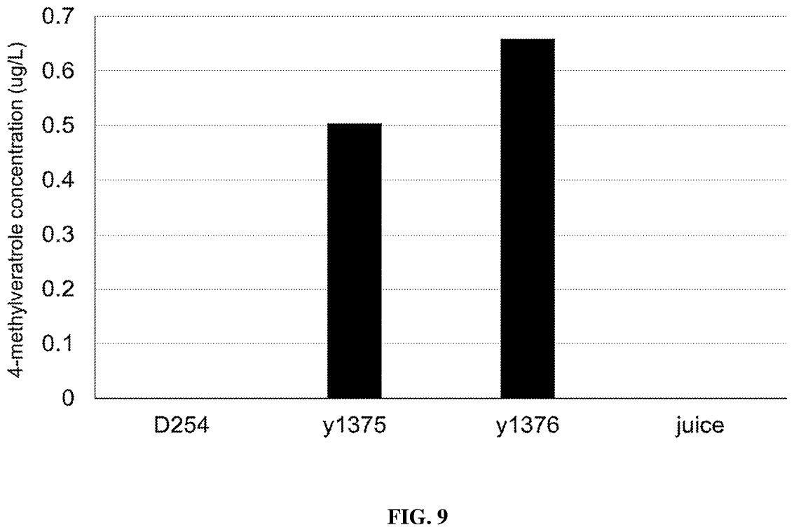

A wine yeast strain referred to as D254, commonly used to make red wines such as Syrah and Pinot Noir, was engineered to constitutively express an O-methyltransferase enzyme derived from basil (EOMT1 from Ocimum basilicum) or loquat (Eriobotrya japonica, EjOMT1), corresponding to engineered strains y1375 and y1376, respectively. Engineered strains y1375 and y1376, along with the parent strain D254, were inoculated into Pinot Noir grape juice containing 500 μg/L of nonvolatile phenolic glucoside substrate (4-methylguaiacol glucoside) and 1.5 μg/mL purified β-glucosidase from almonds. Anaerobic wine fermentation was allowed to proceed for five days at 32° C. The phenolic methyl ether product (4-methylveratrole) was then extracted from the extracellular wine matrix, analyzed by GC/MS, and quantified with authentic standards (FIG. 9).

These data demonstrate that the two enzymatic transformations (non-volatile glycosides to volatile phenols and volatile phenols to phenolic methyl ethers) were successfully carried out with the first enzyme (glucosidase) supplied exogenously and the second enzyme (OMT) expressed in wine yeast. Specifically, during wine fermentation, the added purified β-glucosidase enzyme converted phenolic glucosides into phenols. These phenols were subsequently converted to non-smoky phenolic methyl ethers by genetically modified yeast cells expressing an OMT enzyme. Two expressed OMTs (strain y1375 expressing EjOMT1 from basil and strain y1376 expressing EOMT1 from loquat) exhibited detectable conversion of the smoke taint 4-methylguaicol to 4-methylveratrole.

Small Scale Wine Fermentations with Strains Expressing Both OMT and Secretory β-Glucosidases

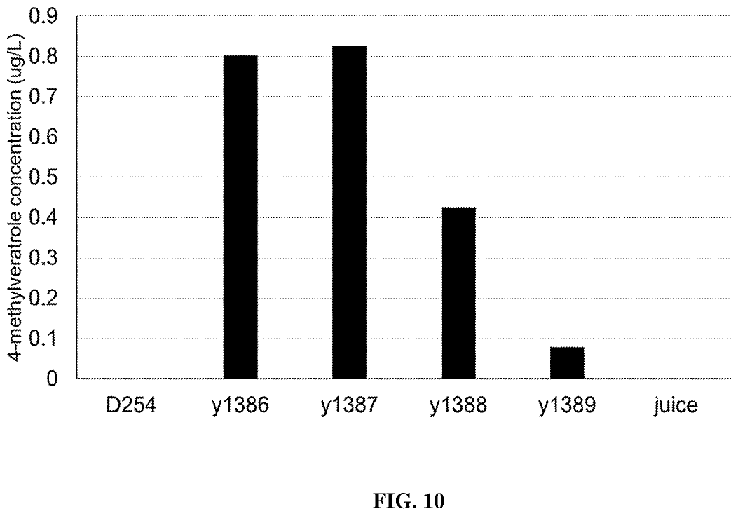

A wine yeast strain referred to as D254, commonly used to make red wines such as Syrah and Pinot Noir, was engineered to constitutively express basil or loquat OMT as well as AoBgl1 fused to a secretion signal peptide (SED1 or TFP5-1), corresponding to engineered strains y1386 (expressing EOMT1 and AoBgl1(N:TFP5-1)), y1387 (expressing EOMT1 and AoBgl1(N:SED1)), y1388 (expressing EjOMT1 and AoBgl1(N:TFP5-1)), and y1389 (expressing EjOMT1 and AoBgl1(N:SED1)) (see, Table 3). Engineered strains y1386, y1387, y1388, and y1389, along with the parent strain D254, were inoculated into Pinot Noir grape juice containing 500 μg/L of nonvolatile phenolic glucoside substrate (4-methylguaiacol glucoside). Anaerobic wine fermentation was allowed to proceed for five days at 32° C. The phenol methyl ether product of the two enzymatic steps (4-methylveratrole) was then extracted from the extracellular wine matrix, analyzed by GC/MS, and quantified with authentic standards (FIG. 10).

These data demonstrate that the two required enzymatic transformations (non-volatile glycosides to volatile phenols and volatile phenols to phenolic methyl ethers) were successfully carried out with both enzymes (glucosidase and OMT) expressed in wine yeast. Specifically, during wine fermentation, engineered yeast strains y1386, y1387, y1388, and y1389 produced the volatile phenolic methyl ether 4-methylveratrole from 4-methylguaiacol glucoside.

What is claimed is:

1. A method of producing a fermented product, the method comprising:
contacting a genetically modified yeast cell (modified cell) with a medium comprising at least one fermentable sugar and one or more non-volatile phenolic glycosides associated with smoke taint, thereby hydrolyzing the one or more non-volatile phenolic glycosides to produce one or more volatile phenols;
wherein the genetically modified cell comprises a heterologous gene encoding an enzyme having glycosidase activity.

2. The method of claim 1, wherein the contacting is performed during at least a first fermentation process to produce a first fermented product comprising volatile phenols.

3. The method of claim 1 or claim 2, further comprising removing one or more volatile phenols from the first fermented product to produce a second fermented product.

4. The method of any one of claims 1-3, wherein the enzyme having glycosidase activity is a glucosidase or a rhamnosidase.

5. The method of claim 4, wherein the glucosidase is a beta-D-glucosidase or the rhamnosidase is an alpha-L-rhamnosidase.

6. The method of any one of claims 1-5, wherein the enzyme having glycosidase activity is derived from Aspergillus aculatus, Aspergillus aculatus, Aspergillus terreus, Aspergillus 25 nidulans, Aspergillus niger, Aspergillus oryzae, Lactobacillus plantarum, Lactobacillus acidophilus, Thermomicrobia PRI-1686, Candida molischiana, Bacillus polymyxa, Olea europaea, Saccharomyces cerevisiae, Thermoascus aurantiacus, Aspergillus niger, Trichoderma reesei, Neurospora crassa, Oenococcus oeni, Prunis dulcis, Bacillus circulans, or Sinorhizobium meliloti.

7. The method of any one of claims 1-6, wherein the enzyme having glycosidase activity is selected from the group consisting of RhaB derived from Aspergillus aculatus, RhaA derived from Aspergillus aculatus, AtRha derived from Aspergillus terreus, AndRha derived from Aspergillus nidulans, AngRha derived from Aspergillus niger, AorhaA derived from Aspergillus oryzae, RhaB1 derived from Lactobacillus plantarum NCC245, RhaB2 derived from Lactobacillus plantarum NCC245, LpRam1 derived from Lactobacillus acidophilus, RhmA derived from Thermomicrobia PRI-1686, RhmB derived from Thermomicrobia PRI-1686, bgln derived from Candida molischiana, bglA derived from Bacillus polymyxa, OepGLU derived from Olea europaea, EGH1 derived from Saccharomyces cerevisiae, Tabgl1 derived from Thermoascus aurantiacus IF09748, AoBGL1 derived from Aspergillus oryzae, AnBGL1 derived from Aspergillus niger, TrBGL2 derived from Trichoderma reesei, TrBGL1 derived from Trichoderma reesei, BGL1 derived from Neurospora crassa, BGL2 derived from Neurospora crassa, bglH derived from Aspergillus oryzae, OoBgl derived from Oenococcus oeni, Ph691 from Prunus dulcis, Ph692 from Prunus dulcis, BsBglA from Bacillus circulans, or SmBgl from Sinorhizobium meliloti.

8. The method of any one of claims 1-7, wherein the enzyme having glycosidase activity comprises a sequence having at least 90% sequence identity to the amino acid sequences set forth in any of SEQ ID NOs: 1-28.

9. The method of claim 8, wherein the enzyme having glycosidase activity comprises the amino acid sequence set forth in any of SEQ ID NOs: 1-28.

10. The method of any one of claims 1-9, wherein the enzyme having glycosidase activity comprises a secretion signal.

11. The method of claim 10, wherein the secretion signal is a peptide selected from the group consisting of SED1, MATα, MATα pre-sequence, TFP5-1, TFP1-4, TFP10, TFP23, SUC2, SRL1, and KSH1.

12. The method of claim 11, wherein the signal sequence comprises the amino acid sequence set forth in any of SEQ ID NOs: 29-36 and 50-54.

13. The method of any one of claims 3-12, wherein removing one or more volatile phenols from the first fermented product comprises filtering the first fermented product.

14. The method of claim 13, wherein the filtering comprises subjecting the first fermented product to reverse osmosis.

15. The method of any one of claims 3-12, wherein removing one or more volatile phenols from the first fermented product comprises contacting the first fermented product with a fining agent.

16. The method of claim 15, wherein the fining agent is activated carbon or a cyclodextrin polymers.

17. The method of any one of claims 3-12, wherein removing one or more volatile phenols from the first fermented product comprises contacting the first fermented product with an enzyme having O-methyltransferase activity.

18. The method of claim 17, wherein the enzyme having O-methyltransferase activity is a recombinant O-methyl transferase enzyme.

19. The method of claim 18, wherein the recombinant O-methyl transferase enzyme is added to the first fermented product.

20. The method of any one of claims 17-19, further comprising adding S-adenosyl methionine to the medium or the first fermented product.

21. The method of any one of claims 1-20, wherein the medium comprises S-adenosyl methionine.

22. The method of claim 17, wherein the enzyme having O-methyltransferase activity is expressed by the modified cell or by a second modified cell.

23. The method of any one of claim 3-12, wherein the modified cell further comprises a second heterologous gene encoding an enzyme having O-methyltransferase activity; and the removing one or more volatile phenols from the first fermented product comprises producing one or more methylated volatile phenols.

24. The method of 23, wherein the second heterologous gene encoding an enzyme having O-methyltransferase activity is derived from Silene latifolia, Solanuma lycopersicum, Rosa hybrida, Ocimum basilicum, Eriobotrya japonica, Pinus taeda, Fragaria ananassa, Schizosaccharomyces pombe, or Zinnia elegans.

25. The method of any one of claims 17-24, wherein the enzyme having O-methyltransferase activity is selected from the group consisting of SIGOMT4 derived from Silene latifolia, SIOMT1 derived from Solanuma lycopersicum, SIOMT4 derived from Solanuma lycopersicum, SICTOMT1 derived from Solanuma lycopersicum, RhOOMT1 derived from Rosa hybrida, RhOOMT2 derived from Rosa hybrida, EOMT1 derived from Ocimum basilicum, EjOMT1 derived from Eriobotrya japonica, AEOMT derived from Pinus taeda, FaOMT derived from Fragaria ananassa, SpCOMT derived from Schizosaccharomyces pombe, COMT1 derived from Ocimum basilicum, and ZeCAOMT derived from Zinnia elegans.

26. The method of any one of claims 17-25, wherein the enzyme having O-methyltransferase activity comprises a sequence having at least 90% sequence identity to the amino acid sequences set forth in any of SEQ ID NOs: 37-49.

27. The method of claim 26, wherein the enzyme having O-methyltransferase activity comprises the amino acid sequence set forth in any of SEQ ID NOs: 37-49.

28. The method of any one of claims 1-27, wherein the one or more non-volatile phenolic glycoside associated with smoke taint comprises a glucoside, a gentiobioside, and/or a rutinoside.

29. The method of any one of claims 1-28, wherein one or more volatile phenol comprises guaiacol, m-cresol, p-cresol, o-cresol, phenol, 4-methylguaiacol, syringol, and/or 4-methylsyringol.

30. The method of any one of claims 23-29, wherein the one or more methylated volatile phenols comprises veratrole (1,2-dimethoxybenzene), 3-methylanisole, and/or 4-methylveratrole.

31. The method of any one of claims 1-30, wherein the yeast cell is of the genus Saccharomyces.

32. The method of claim 31, wherein the yeast cell is of the species Saccharomyces cerevisiae (S. cerevisiae).

33. The method of claim 32, wherein the yeast cell is S. cerevisiae California Ale Yeast strain WLP001, EC-1118, Elegance, Red Star Côte des Blancs, Epernay II, London Ale III, Augustiner, W-34/70, Andechs, D254, RC212, or B0213.

34. The method of claim 31, wherein the yeast cell is of the species Saccharomyces pastorianus (S. pastorianus).

35. The method of any one of claims 1-34, wherein at least one fermentable sugar is provided in at least one sugar source.

36. The method of claim 35, wherein the fermentable sugar is glucose, fructose, sucrose, maltose, and/or maltotriose.

37. The method of any one of claims 1-36, wherein the fermented product comprises a reduced level of one or more non-volatile phenolic glycosides as compared to a fermented product produced by a counterpart cell that does not express the heterologous gene.

38. The method of any one of claims 1-37, wherein the fermented product is a fermented beverage.

39. The method of claim 38, wherein the fermented beverage is beer, wine, sparkling wine (champagne), wine cooler, wine spritzer, hard seltzer, sake, mead, kombucha, or cider.

40. The method of any one of claims 35-39, wherein the sugar source comprises wort, must, fruit juice, honey, rice starch, or a combination thereof.

41. The method of claim 40, wherein the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

42. The method of claim 40, wherein the sugar source is wort and the method further comprises producing the medium, wherein producing the medium comprises:
(a) contacting a plurality of grains with water; and
(b) boiling or steeping the water and grains to produce wort.

43. The method of claim 42, further comprising adding at least one hop variety to the wort to produce a hopped wort.

44. The method of any one of claims 1-43, further comprising adding at least one hop variety to the medium.

45. The method of claim 40, wherein the sugar source is must and the method further comprises producing the medium, wherein producing the medium comprises crushing a plurality of fruit to produce the must.

46. The method of claim 45, further comprising removing solid fruit material from the must to produce a fruit juice.

47. The method of any one of claims 1-46, further comprising at least one additional fermentation process.

48. The method of any one of claims 1-47, further comprising carbonating the fermented product.

49. A genetically modified yeast cell (modified cell) comprising:
(i) a first heterologous gene encoding an enzyme having glycosidase activity, and
(ii) a second heterologous gene encoding an enzyme having O-methyltransferase activity.

50. The modified cell of claim 49, wherein the enzyme having glycosidase activity is a glucosidase or a rhamnosidase.

51. The modified cell of claim 50, wherein the glucosidase is a beta-D-glucosidase or the rhamnosidase is an alpha-L-rhamnosidase.

52. The modified cell of any one of claims 49-51, wherein the enzyme having glycosidase activity is derived from Aspergillus aculatus, Aspergillus aculatus, Aspergillus terreus, Aspergillus nidulans, Aspergillus niger, Aspergillus oryzae, Lactobacillus plantarum, Lactobacillus acidophilus, Thermomicrobia PRI-1686, Candida molischiana, Bacillus polymyxa, Olea europaea, Saccharomyces cerevisiae, Thermoascus aurantiacus, Aspergillus niger, Trichoderma reesei, Neurospora crassa, Oenococcus oeni, Prunis dulcis, Bacillus circulans, or Sinorhizobium meliloti.

53. The modified cell of claim 52, wherein the enzyme having glycosidase activity is selected from the group consisting of RhaB derived from Aspergillus aculatus, RhaA derived from Aspergillus aculatus, AtRha derived from Aspergillus terreus, AndRha derived from Aspergillus nidulans, AngRha derived from Aspergillus niger, AorhaA derived from Aspergillus oryzae, RhaB1 derived from Lactobacillus plantarum NCC245, RhaB2 derived from Lactobacillus plantarum NCC245, LpRam1 derived from Lactobacillus acidophilus, RhmA derived from Thermomicrobia PRI-1686, RhmB derived from Thermomicrobia PRI-1686, bgln derived from Candida molischiana, bglA derived from Bacillus polymyxa, OepGLU derived from Olea europaea, EGH1 derived from Saccharomyces cerevisiae, Tabgl1 derived from Thermoascus aurantiacus IF09748, AoBGL1 derived from Aspergillus oryzae, AnBGL1 derived from Aspergillus niger, TrBGL2 derived from Trichoderma reesei, TrBGL1 derived from Trichoderma reesei, BGL1 derived from Neurospora crassa, BGL2 derived from Neurospora crassa, bglH derived from Aspergillus oryzae, OoBgl derived from Oenococcus oeni, Ph691 from Prunus dulcis, Ph692 from Prunus dulcis, BsBglA from Bacillus circulans, or SmBgl from Sinorhizobium meliloti.

54. The modified cell of any one of claims 49-53, wherein the enzyme having glycosidase activity comprises a sequence having at least 90% sequence identity to the amino acid sequences set forth in any of SEQ ID NOs: 1-28.

55. The modified cell of claim 54, wherein the enzyme having glycosidase activity comprises the amino acid sequence set forth in any of SEQ ID NOs: 1-28.

56. The modified cell of any one of claims 49-55, wherein the enzyme having glycosidase activity comprises a secretion signal.

57. The modified cell of claim 56, wherein the secretion signal is a peptide selected from the group consisting of SED1, MATα, MATα pre-sequence, TFP5-1, TFP1-4, TFP10, TFP23, SUC2, SRL1, and KSH1.

58. The modified cell of claim 57, wherein the signal sequence comprises the amino acid sequence set forth in any of SEQ ID NOs: 29-36 and 50-54.

59. The modified cell of any one of claims 49-58, wherein the second heterologous gene encoding an enzyme having O-methyltransferase activity is derived from Silene latifolia, Solanuma lycopersicum, Rosa hybrida, Ocimum basilicum, Eriobotrya japonica, Pinus taeda, Fragaria ananassa, Schizosaccharomyces pombe, or Zinnia elegans.

60. The modified cell of any one of claims 49-59, wherein the enzyme having O-methyltransferase activity is selected from the group consisting of SIGOMT4 derived from Silene latifolia, SIOMT1 derived from Solanuma lycopersicum, SIOMT4 derived from Solanuma lycopersicum, SICTOMT1 derived from Solanuma lycopersicum, RhOOMT1 derived from Rosa hybrida, RhOOMT2 derived from Rosa hybrida, EOMT1 derived from Ocimum basilicum, EjOMT1 derived from Eriobotrya japonica, AEOMT derived from Pinus taeda, FaOMT derived from Fragaria ananassa, SpCOMT derived from Schizosaccharomyces pombe, COMT1 derived from Ocimum basilicum, and ZeCAOMT derived from Zinnia elegans.

61. The modified cell of claim 60, wherein the enzyme having O-methyltransferase activity comprises a sequence having at least 90% sequence identity to the amino acid sequences set forth in any of SEQ ID NOs: 37-49.

62. The modified cell of claim 61, wherein the enzyme having O-methyltransferase activity comprises the amino acid sequence set forth in any of SEQ ID NOs: 37-49.

63. The modified cell of any one of claims 49-62, wherein the yeast cell is of the genus Saccharomyces.

64. The modified cell of claim 63, wherein the yeast cell is of the species Saccharomyces cerevisiae (S. cerevisiae).

65. The modified cell of claim 64, wherein the yeast cell is S. cerevisiae California Ale Yeast strain WLP001, EC-1118, Elegance, Red Star Côte des Blancs, Epernay II, London Ale III, Augustiner, W-34/70, Andechs, D254, RC212, or B0213.

66. The modified cell of claim 63, wherein the yeast cell is of the species Saccharomyces pastorianus (S. pastorianus).

67. A method of producing a fermented product comprising,
contacting the modified cell of any one of claims 49-66 with a medium comprising at least one fermentable sugar,
wherein the contacting is performed during at least a first fermentation process, to produce a fermented product.

68. The method of claim 67, wherein the medium comprises one or more non-volatile phenolic glycoside associated with smoke taint.

69. The method of claim 68, wherein the non-volatile phenolic glycoside associated with smoke taint comprises a glucoside, a gentiobioside, and/or a rutinoside.

70. The method of any one of claims 67-69, wherein the fermented product comprises one or more methylated volatile phenols.

71. The method of claim 70, wherein the one or more methylated volatile phenols comprises veratrole (1,2-dimethoxybenzene), 3-methylanisole, and/or 4-methylveratrole

72. The method of any one of claims 67-71, wherein at least one fermentable sugar is provided in at least one sugar source.

73. The method of claim 72, wherein the fermentable sugar is glucose, fructose, sucrose, maltose, and/or maltotriose.

74. The method of any one of claims 67-73, wherein the fermented product comprises a reduced level of one or more non-volatile phenolic glycosides as compared to a fermented product produced by a counterpart cell that does not express the first heterologous gene and/or second heterologous gene.

75. The method of any one of claims 67-74, wherein the fermented product is a fermented beverage.

76. The method of claim 75, wherein the fermented beverage is beer, wine, sparkling wine (champagne), wine cooler, wine spritzer, hard seltzer, sake, mead, kombucha, or cider.

77. The method of any one of claims 72-76, wherein the sugar source comprises wort, must, fruit juice, honey, rice starch, or a combination thereof.

78. The method of claim 77, wherein the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

79. The method of claim 77, wherein the sugar source is wort and the method further comprises producing the medium, wherein producing the medium comprises:
(a) contacting a plurality of grains with water; and
(b) boiling or steeping the water and grains to produce wort.

80. The method of claim 79, further comprising adding at least one hop variety to the wort to produce a hopped wort.

81. The method of any one of claims 67-80, further comprising adding at least one hop variety to the medium.

82. The method of claim 77, wherein the sugar source is must and the method further comprises producing the medium, wherein producing the medium comprises crushing a plurality of fruit to produce the must.

83. The method of claim 82, further comprising removing solid fruit material from the must to produce a fruit juice.

84. The method of any one of claims 67-83, further comprising at least one additional fermentation process.

85. The method of any one of claims 67-84, further comprising carbonating the fermented product.

86. A method of producing a composition comprising ethanol comprising,
contacting the modified cell of any one of claims 49-66 with a medium comprising at least one fermentable sugar,
wherein the contacting is performed during at least a first fermentation process, to produce a composition comprising ethanol.

87. The method of claim 86, wherein the medium comprises one or more non-volatile phenolic glycoside associated with smoke taint.

88. The method of claim 87, wherein the non-volatile phenolic glycoside associated with smoke taint comprises a glucoside, a gentiobioside, and/or a rutinoside.

89. The method of any one of claims 86-88, wherein the composition further comprises one or more methylated volatile phenols.

90. The method of claim 89, wherein the one or more methylated volatile phenols comprises veratrole (1,2-dimethoxybenzene), 3-methylanisole, and/or 4-methylveratrole.

91. The method of any one of claims 86-90, wherein at least one fermentable sugar is provided in at least one sugar source.

92. The method of claim 91, wherein the fermentable sugar is glucose, fructose, sucrose, maltose, and/or maltotriose.

93. The method of any one of claims 86-92, wherein composition comprises a reduced level of one or more non-volatile phenolic glycosides as compared to a composition produced by a counterpart cell that does not express the first heterologous gene and/or second heterologous gene.

94. The method of any one of claims 86-93, wherein the composition is a fermented beverage.

95. The method of claim 94, wherein the fermented beverage is beer, wine, sparkling wine (champagne), wine cooler, wine spritzer, hard seltzer, sake, mead, kombucha, or cider.

96. The method of any one of claims 91-95, wherein the sugar source comprises wort, must, fruit juice, honey, rice starch, or a combination thereof.

97. The method of claim 96, wherein the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

98. The method of claim 96, wherein the sugar source is wort and the method further comprises producing the medium, wherein producing the medium comprises:
(a) contacting a plurality of grains with water; and
(b) boiling or steeping the water and grains to produce wort.

99. The method of claim 98, further comprising adding at least one hop variety to the wort to produce a hopped wort.

100. The method of any one of claims 86-99, further comprising adding at least one hop variety to the medium.

101. The method of claim 96, wherein the sugar source is must and the method further comprises producing the medium, wherein producing the medium comprises crushing a plurality of fruit to produce the must.

102. The method of claim 101, further comprising removing solid fruit material from the must to produce a fruit juice.

103. The method of any one of claims 86-102, further comprising at least one additional fermentation process.

104. The method of any one of claims 86-103, further comprising carbonating the composition.

105. A method of producing a fermented product, the method comprising:
contacting a genetically modified yeast cell (modified cell) with a medium comprising at least one fermentable sugar and one or more volatile phenols, thereby converting the one or more volatile phenols to one or more methylated volatile phenols;
wherein the genetically modified cell comprises a heterologous gene encoding an enzyme having O-methyltransferase activity.

106. The method of claim 105, wherein the medium further comprises one or more non-volatile phenolic glycosides associated with smoke taint.

107. The method of claim 105 or 106, further comprising contacting the medium with an enzyme having glycosidase activity thereby producing the one or more volatile phenols.

108. The method of claim 107, wherein the enzyme having glycosidase activity is a recombinant and/or purified glycosidase enzyme.

109. The method of claim of 107 or 108, wherein the enzyme having glycosidase activity is a glucosidase or a rhamnosidase.

110. The method of claim 109, wherein the glucosidase is a beta-D-glucosidase or the rhamnosidase is an alpha-L-rhamnosidase.

111. The method of any one of claims 107-110, wherein the enzyme having glycosidase activity is a recombinant and/or purified glycosidase enzyme obtained from almonds.

112. The method of any one of claims 107-110, wherein the enzyme having glycosidase activity is derived from Aspergillus aculatus, Aspergillus aculatus, Aspergillus terreus, Aspergillus nidulans, Aspergillus niger, Aspergillus oryzae, Lactobacillus plantarum, Lactobacillus acidophilus, Thermomicrobia PRI-1686, Candida molischiana, Bacillus polymyxa, Olea europaea, Saccharomyces cerevisiae, Thermoascus aurantiacus, Aspergillus niger, Trichoderma reesei, Neurospora crassa, Oenococcus oeni, Prunis dulcis, Bacillus circulans, or Sinorhizobium meliloti.

113. The method of any one of claims 107-110 or 112, wherein the enzyme having glycosidase activity is selected from the group consisting of RhaB derived from Aspergillus aculatus, RhaA derived from Aspergillus aculatus, AtRha derived from Aspergillus terreus, AndRha derived from Aspergillus nidulans, AngRha derived from Aspergillus niger, AorhaA derived from Aspergillus oryzae, RhaB1 derived from Lactobacillus plantarum NCC245, RhaB2 derived from Lactobacillus plantarum NCC245, LpRam1 derived from Lactobacillus acidophilus, RhmA derived from Thermomicrobia PRI-1686, RhmB derived from Thermomicrobia PRI-1686, bgln derived from Candida molischiana, bglA derived from Bacillus polymyxa, OepGLU derived from Olea europaea, EGH1 derived from Saccharomyces cerevisiae, Tabgl1 derived from Thermoascus aurantiacus IF09748, AoBGL1 derived from Aspergillus oryzae, AnBGL1 derived from Aspergillus niger, TrBGL2 derived from Trichoderma reesei, TrBGL1 derived from Trichoderma reesei, BGL1 derived from Neurospora crassa, BGL2 derived from Neurospora crassa, bglH derived from Aspergillus oryzae, OoBgl derived from Oenococcus oeni, Ph691 from Prunus dulcis, Ph692 from Prunus dulcis, BsBglA from Bacillus circulans, or SmBgl from Sinorhizobium meliloti.

114. The method of any one of claims 107-110, 112, or 113, wherein the enzyme having glycosidase activity comprises a sequence having at least 90% sequence identity to the amino acid sequences set forth in any of SEQ ID NOs: 1-28.

115. The method of claim 114, wherein the enzyme having glycosidase activity comprises the amino acid sequence set forth in any of SEQ ID NOs: 1-28.

116. The method of any one of claims 105-115, wherein the enzyme having O-methyltransferase activity is derived from Silene latifolia, Solanuma lycopersicum, Rosa hybrida, Ocimum basilicum, Eriobotrya japonica, Pinus taeda, Fragaria ananassa, 5 Schizosaccharomyces pombe, or Zinnia elegans.

117. The method of any one of claims 105-116, wherein the heterologous gene encoding an enzyme having O-methyltransferase activity is derived from Silene latifolia, Solanuma lycopersicum, Rosa hybrida, Ocimum basilicum, Eriobotrya japonica, Pinus taeda, Fragaria ananassa, Schizosaccharomyces pombe, or Zinnia elegans.

118. The method of any one of claims 105-117, wherein the enzyme having O-methyltransferase activity is selected from the group consisting of SIGOMT4 derived from Silene latifolia, SIOMT1 derived from Solanuma lycopersicum, SIOMT4 derived from Solanuma lycopersicum, SICTOMT1 derived from Solanuma lycopersicum, RhOOMT1 derived from Rosa hybrida, RhOOMT2 derived from Rosa hybrida, EOMT1 derived from Ocimum basilicum, EjOMT1 derived from Eriobotrya japonica, AEOMT derived from Pinus taeda, FaOMT derived from Fragaria ananassa, SpCOMT derived from Schizosaccharomyces pombe, COMT1 derived from Ocimum basilicum, and ZeCAOMT derived from Zinnia elegans.

119. The method of claim 105-118, wherein the enzyme having O-methyltransferase activity comprises a sequence having at least 90% sequence identity to the amino acid sequences set forth in any of SEQ ID NOs: 37-49.

120. The method of claim 119, wherein the enzyme having O-methyltransferase activity comprises the amino acid sequence set forth in any of SEQ ID NOs: 37-49.

121. The method of any one of claims 105-120, wherein the enzyme having O-methyltransferase activity comprises a secretion signal.

122. The method of claim 121, wherein the secretion signal is a peptide selected from the group consisting of SED1, MATα, MATα pre-sequence, TFP5-1, TFP1-4, TFP10, TFP23, SUC2, SRL1, and KSH1.

123. The method of claim 122, wherein the signal sequence comprises the amino acid sequence set forth in any of SEQ ID NOs: 29-36 and 50-54.

124. The method of any one of claims 105-121, wherein one or more volatile phenol comprises guaiacol, m-cresol, p-cresol, o-cresol, phenol, 4-methylguaiacol, syringol, and/or 4-methylsyringol.

125. The method of any one of claims 105-124, wherein the one or more methylated volatile phenols comprises veratrole (1,2-dimethoxybenzene), 3-methylanisole, and/or 4-methylveratrole.

126. The method of any one of claims 106-123, wherein the one or more non-volatile phenolic glycoside associated with smoke taint comprises a glucoside, a gentiobioside, and/or a rutinoside.

127. The method of any one of claims 105-126, wherein the yeast cell is of the genus Saccharomyces.

128. The method of claim 127, wherein the yeast cell is of the species Saccharomyces cerevisiae (S. cerevisiae).

129. The method of claim 128, wherein the yeast cell is S. cerevisiae California Ale Yeast strain WLP001, EC-1118, Elegance, Red Star Côte des Blancs, Epernay II, London Ale III, Augustiner, W-34/70, Andechs, D254, RC212, or B0213.

130. The method of claim 127, wherein the yeast cell is of the species Saccharomyces pastorianus (S. pastorianus).

131. The method of any one of claims 105-130, wherein at least one fermentable sugar is provided in at least one sugar source.

132. The method of claim 131, wherein the fermentable sugar is glucose, fructose, sucrose, maltose, and/or maltotriose.

133. The method of any one of claims 105-132, wherein the fermented product comprises a reduced level of one or more volatile phenols as compared to a fermented product produced by a counterpart cell that does not express the heterologous gene.

134. The method of any one of claims 106-133, wherein the fermented product comprises a reduced level of one or more non-volatile phenolic glycosides as compared to a fermented product produced by a counterpart cell that does not express the heterologous gene.

135. The method of any one of claims 105-134, wherein the fermented product is a fermented beverage.

136. The method of claim 135, wherein the fermented beverage is beer, wine, sparkling wine (champagne), wine cooler, wine spritzer, hard seltzer, sake, mead, kombucha, or cider.

137. The method of any one of claims 131-136, wherein the sugar source comprises wort, must, fruit juice, honey, rice starch, or a combination thereof.

138. The method of claim 137, wherein the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

139. The method of claim 137, wherein the sugar source is wort and the method further comprises producing the medium, wherein producing the medium comprises:
(a) contacting a plurality of grains with water; and
(b) boiling or steeping the water and grains to produce wort.

140. The method of claim 139, further comprising adding at least one hop variety to the wort to produce a hopped wort.

141. The method of any one of claims 105-140, further comprising adding at least one hop variety to the medium.

142. The method of claim 137, wherein the sugar source is must and the method further comprises producing the medium, wherein producing the medium comprises crushing a plurality of fruit to produce the must.

143. The method of claim 142, further comprising removing solid fruit material from the must to produce a fruit juice.

144. The method of any one of claims 105-143, further comprising at least one additional fermentation process.

145. The method of any one of claims 105-144, further comprising carbonating the fermented product.


SEQUENCE LISTING (54 sequences)

SEQ ID NO: 1 - protein, 597 aa, unidentified
  source 1..597: note = Aspergillus aculeatus
MHIITPLLIPAVLVAAARVPYREYILAPSSRVIVPASVRQVNGSVTNAAGLTGSSLGTAVFHGVSSVTYDFGKNVAGIVSLTVGSSSSPSAFLGVTFSESSLWASSEACDATGNSGLDAPLWFPVGQKAGTYTPDSKYVRGGFRYLTVVSNTSATIPLNSLHITFTAAPDQDLQAYQGWFHSNDELLNEIWYAGAYTNQLCTIDPTYGSASSETISTSGLNYWYNNLTIANGTSTVTDGAKRDRAVWPGDMSISLESIAVSTNDLYSVRMGLEALLALQSSEGQLPWGGKPFNIDVSYTYHLHSLIGMSFLYRFSGDKVWLSNYWGQYSKGVEWAVRSVADGVKSAANQLLWDDQAGLYRDNQTTELHPQDGNAWAVKSNLTLSGSQNRAISQALKARWGRYGAPAPEAGATISPFIGGFEIQSHFLANQPDVALDMIRLQWGFMLRDPRMTQSTLIEGYSTDGSIHYAPYANDARISHAHGWSTGPTYALTAYAAGLQLLGPAGNSWLIAPQPGGLTSIDCGFATALGVFSVVFERDSVGRYNSFSFGAPTGTTGRIELPGVRGTLVSTTGQRVQLVNGTASGLRGGKWKLIESAD

SEQ ID NO: 2 - protein, 660 aa, unidentified
  source 1..660: note = Aspergillus aculeatus
MLWSSWILTPALLAIGSHAVPFEDYILAPQSRTLNPSLVYQVNGTVDNPEALVGLTTHQTTLHGKSSVTYDFGRNIAGIVSLDVTSVSSKAAQLGVTFTESSLWISSEACDATSDAGLDSPLWFSVGHGPGTYGADKKHLRGAFRYLTLVNNSTATISLDGLSINYTAAPTQDLRGYKGYFHSSDELINRIWYAGAYTLQLCTIDPTTGDSLIWLGVISSSDNITLPQTDSWWNNYTITNGSTTLVDGAKRDRLVWPGDMSIAIESAAVSTADLESVRTALESLFVLQKANGQLPYAGRPFLDIVSFTYHLHSLIGASSYYQYTGDRSWITRYWGQYKKGLQWALSSLDNSGLANITASADWLRFGMGGHNIEANAILYYVLNDAISLASSLDDRANVGNWSTAASKIKAAANARLWDAQNSLYRDNETTTLHPQDGNAWAIKANLTLSSNQSEAISSALAARWGPYGAPAPEAGSTVSPFIGGFELQAHYLANEPDRALDLLRLQWGFMLDDPRMTNSTFIEGYSTDGSLAYAPYRNTPRVSHAHGWSTGPTSALTHYTAGLRLTGPAGSTWLFKPQPGNLTEVQAGFETQLGLFATQYQKSATGTFQQLTFTAPNGTSGSVEIEGATGQLISKRGQAVKLVNGKARGLQGGTWTLKGL

SEQ ID NO: 3 - protein, 871 aa, unidentified
  source 1..871: note = Aspergillus terreus
MALSISQVAFEHHRTALGIGETQPRVSWRFDGNVSDWEQRAYEIEVKRAGHDADVFRSESSDSVLVPWPSSPLQSGEEATVRVRSFGSDGQHDTPWSDAVTVEPGLLTPDDWHDAVVIASDRPTEVDATHRPIQFRKEFSVDDSYVSARLYITALGLYEARINDQRVGDHVMAPGWQSYQYRHEYNTYDVTDLLKQGPNAIGVTVGEGWYSGRIGYDGGKRNIYGDTLGLLSLLVVTKSDGSKLYIPSDSSWKSSTGPIISSEIYDGEEYDSRLEQKGWSQVGFNSTGWLGTHELSFPKERLASPDGPPVRRVAEHKLANVFSSASGKTVLDFGQNLVGWLRIRVKGPKGQTIRFVHTEVMENGEVATRPLRQAKATDHFTLSGEGVQEWEPSFTYHGFRYVQVDGWPADTPLDENSVTAIVVHSDMERTGYFECSNPLISKLHENILWSMRGNFFSIPTDCPQRDERLGWTGDIHAFSRTANFIYDTAGFLRAWLKDARSEQLNHSYSLPYVIPNIHGNGETPTSIWGDAIVGVPWQLYESFGDKVMLEEQYGGAKDWVDKGIVRNDVGLWDRSTFQWADWLDPKAPADDPGDATTNKYLVSDAYLLHSTDMLANISTSLSKGEEASNYTEWHAKLTKEFQKAWITSNGTMANETQTGLALPLYFDLFPSAEQAQSAAKRLVNIIKQNDYKVGTGFAGTHLLGHTLSKYGESDAFYSMLRQTEVPSWLYQVVMNGTTTWERWDSMLPNGSINPGQMTSFNHYAVGSVGSWLHEVIGGLSPAEPGWRRINIEVVPGGDLQQASTKFLTPYGMASTKWWLDGQDQSCGGFDFHLVAEVPPNTRATVVLPGKGGEKVDVGSGVHEYHVRCVKL

SEQ ID NO: 4 - protein, 661 aa, unidentified
  source 1..661: note = Aspergillus nidulans
MISLPLLALATGAVASASCWRNTSCTGPSKPSFPGPWDSNNFAPGSRLIQPKSILSLPDGTYISDYTSDSHPSITTEDAGLVFDFGLEVGGIVTIDYSSSVPNTTLGLAFTEAKDYIGRTSDNSNGGTGADGALATLLTEGEGSYTMPDSQLRGGFRYLALFIPSSSSSNASLTIQSITLELAFQPTWSNLRAYQGYFHSSDPVLNKAWYAGAYTLQTNSVPRTTCRVSVSSATGWNNNAVCGPGETLLLDGAKRDRWVWIGDMGVAVPSASVSTGDTESTKNALLAIWDNQTPSGLLPKAGPPYLRADSDTYHLWTIIGTYNYFLFTEDYDFLAGIWQKYVKALDYSLAKITPLGIMNATQTADWGRWNYGTLASSANMLLYRSLTTAAFLAPYAGDNPENYTDLASTLRSAIVTHLYDSAVGAFRDSPNSTLYPQDANSMALAFSFFSQSPLNCSSSFNLAEAARVSSYLESNWTPIGPEVPELPNNISPFISSIELEGHFASGHADRAIELIRMLWGWYLAHPNGTQSTVPEGYLVDGSWGYRGDRGYRNDPRYVSHAHGWSSGPTSTLTEYAVGLKITKPKGSEWSLRPASFGIQGFEEAQAGFTTGLGKFKAAFRVQGKRATVTWDTPAGTKGWVQLPGEEGSWVEGGMGSLTVKL

SEQ ID NO: 5 - protein, 861 aa, unidentified
  source 1..861: note = Aspergillus nidulans
MSLSISGVTFEHHRSALGIGEPSPRISWRFDGTVSNWTQSAYEIEINRAGQANTFRVNSSDSVLVPWPSDPLQSGEEATVRVRSFGRANQPDAPWSDPVTVEPGLLDEDDWQSAVAIVSDRETEVNATHRPIYFRKDFDVDEEILSARLYITALGVYEAEINGQPVGDHVLAPGWQAYSHRHEYNTYDVTDLLQTGDNTIGVTVGEGWYAGALTWSMTRNIYGDTLGLLSLLSIATADGKTIYVPSDETWQSSTGPIIASEIYNGETYDSTQAIEGWSQPGFDASGWLGTHEVTFDKSVLAAPDAPAVRRVEERRLESVFKSASGKTVLDFGQNLVGWLRVRVKGPRGSTISFVHTEVMENGEVATRPLRNAKATDNLTLSGEEQEWEPSFTFHGFRYVQVTGWPEETELNADSVTAIVINSDMEQTGFFSCSNPLLNKLHENIIWSMRGNFLSIPTDCPQRDERLGWTGDIHAFARTANFIYDTSGFLRGWLRDAYSEQLENNYAPPYVIPNVLGPGSPTSIWGDAIVSVPWDLFQTYGDKAMLSEQYAGATAWLDKGILRNEAGLWNRSTFQYADWLDPLAPPDDPGAATTNKYLVSDAYLIHSTELVANISAYLDRPDDAERYAADRADLTRAFQKAWISANGTVANETQTGLTLPLYFKLFERPEHYTDAVSRLVDIIKENEYKVGTGFAGTHLLGHTLSAYNASSTFYNTLLQEDVPGWLFQVLMNGTTTWERWDSMLANGSVNPGEMTSFNHYAVGSVGAWMHENIGGLRPIEPGWRRFAVDVKVGGGLSSAQERFLSPYGSAESSWEVRDGKFMLGVKVPPNSEAVVSLPGAPTRGKKEVIVGSGMHRFESTLG

SEQ ID NO: 6 - protein, 656 aa, unidentified
  source 1..656: note = Aspergillus niger
MWSSWLLSALLATEALAVPYEEYILAPSSRDLAPASVRQVNGSVTNAAALTGAGGQATFNGVSSVTYDFGINVAGIVSVDVASASSDSAFIGVTFTESSMWISSEACDATQDAGLDTPLWFAVGQGAGLYTVEKKYNRGAFRYMTVVSNTTATVSLNSVKINYTASPTQDLRAYTGYFHSNDELLNRIWYAGAYTLQLCSIDPTTGDALVGLGVITSSETISLPQTDKWWTNYTITNGSSTLTDGAKRDRLVWPGDMSIALESVAVSTEDLYSVRTALESLYALQKPDGRLPYAGKPFFDTVSFTYHLHSLVGAASYYQYTGDRAWLTRYWGQYKKGVQWALSSVDSTGLANITASADWLRFGMGAHNIEANAILYYVLNDAISLAQTLNDNAPIRNWTTTAARIKTVANELLWDDKNGLYTDNETTTLHPQDGNSWAVKANLTLSANQSAIVSESLAARWGPYGAPAPEAGATVSPFIGGFELQAHYQAGQPDRALDLLRLQWGFMLDDPRMTNSTFIEGYSTDGSLAYAPYTNTPRVSHAHGWATGPTSALTIYTAGLRVTGPAGATWLYKPQPGNLTQVEAGFSTRLGSFASSFSRSGGRYQELSFSTPNGTTGSVELGDVSGQLVSDRGVKVQLVGGKASGLQGGKWKLSNN

SEQ ID NO: 7 - protein, 663 aa, unidentified
  source 1..663: note = Aspergillus oryzae
MRLPWSAWLLSPALLAVACYGVPYNEYILAPASRHLVPFEVLEVNGSVTDPSSLTQSTGGNATFNGPASVTFDFGRNIAGIVSLDIGSSSTRDAFIGVTFTESSLWISSQACDATADSGLDSPLWFPVGRGAGTYTADKKHNRGAFRYMTLVTNTTAVVSVRNVQINYTAAPSQDLRAYTGYFHSNDELLNRVWYAGAYTNQICTIDPSTGDALPFLGVISSDSNITLPETNPWYSNYTISNGSSTLTDGAKRDRLIWPGDMSIALESVSVSTADLYSVRTALETLLSQQRSDGRLPYASEPFLDLVSYTYHLHSLIGVSYYYRHSGDRAWLSKYWGQYQKGLQWALSSVDNTGLANITASSDWLRFGMGGHNIEANAILYFVLNEAQELSQAINNHTNANWTKIASGIKSATNKNLWDANNGLYKDNETTTLYPQDGNAWAIKANLTLSTNQSSTISSALSSRWGNYGAPAPEAADAVSPFIGGFEIQAHFLANQPQKALDLIRLQWGFMLDDPRMTNSTFIEGYSTDGTLHYAPYTNDARVSHAHGWSTGPTAALSFFVAGLHLTGSAGATWRFAPQPGDLTSVDAGYTTALGLFSTTFKRSENGDYQELTFTTPQGTTGDVDLAGAEGTLVSADGERVFLVKGTATGITGGSWNLEVASQ

SEQ ID NO: 8 - protein, 650 aa, unidentified
  source 1..650: note = Lactobacillus pantarum NCC245
MSKEAVWLWYPGDFEIHQGMLQNFKREERGMGWPAYWYIDDCNRNVNFKRHYDLKESTQFTVLAKGTGYVDVNGTKYRLNHAINCDAGATDIQVFVGNVQGLPTIYIVGEIIKSDSGWLASNFVTTLPAGHDILYTDRNQDPNVIEYRTEKVVAKAQQAVDGGVLYDFGRAVNGTVTVKTNGPVTLCYGESETEARDVEMCYYKQSDVTATTKVRKRAFRYVFVPHCQLGDIELTAMHEYIPKNNPSSFTSDNKLINQIWNVATETLNLCSDLFFIDGIKRDRWIWAGDAYQANFINQYSFFNEDIDKRTLLALRGQDDIKQHMNTIVDYSMLWVIGVLNHYQMTGDREFLKIIYPKLESMVQYFIQQTNEHGFIYGRKNDWIFVDWSEMDKQGTVAAEQILLLEDYKTIMTCGEVLGKDVAGYQAKYDQLFKNLMKYFWDDEQGAFIDSYESGKRHVTRHANIFAILFDVVDENKQQLILKNVLLNNAITQITTPYFKFFEQDALCKLGEQHRVYQVLLDYWGGMLDRGAVTFWEEFDPSQHGKDMYAMYGDPYGKSLCHAWGASPIYLLGRHFVGLRPTAPGYQTFEIKPELSEFHHLHTVLPIKGGTVTVVKDQHQLSVTASRAGGTLIVDGQRQSLEPNRTAVVPV

SEQ ID NO: 9 - protein, 525 aa, unidentified
  source 1..525: note = Lactobacillus pantarum NCC245
MAFTFQINNDVQFRHNQALLDKSASYRPILKETQVKAASIVALELDTQYLEGWGVKQIAPIERLSSYELKRDDQIIIDFGDHQVGQFSININAVGSPMDAPLCFKIKFAEMPAELARKSEDYDGWLSKSWIQEETVHLDVLPTTLTLPRRYSFRYAEITVVDTSPKWRAVFSNPVVTATSAVDTATVHQPELADAQLQRIYEVGLKTLADCMQDVFEDGPKRDRRLWIGDLRLQALANYATFKDTDLVKRCLYLFGAMPTTAGRIPANVFTKPTAVPDDTFLFDYSLFFISILADYEAFSSDKTVLNDLYRVAKNQMDLALAQVTSEGKLKLTEENPVFIDWSNDFDKETAGQAIIIYTLKQFITLAELVNDTSLETYTAILRKLNQYAKTQLFDSQSGLFVSGDQREVNVASQVWMTLAHVLDPEQTTALMQTTVTKLFPITGIATPYMYHHITEALFEAGLKQEAVQLMKDYWGKMITLGADTYWEAFDPNQPDYSPYGSPILNSYCHAWSCTPVYLINKYLV

SEQ ID NO: 10 - protein, 932 aa, unidentified
  source 1..932: note = Thermomicrobia PRI-1686
MLRIDRVKVERSRDGLGLGTGRPRLCWRVETDIRDWRQAAYEVELYDGSGQLVGSTGRVESGESVWVAWPFEALGSRQRAGVRVRVWGEDGSESDWSDLQWLEVGLLARDDWQGAFITPDWEEDTSVANPCPYLRKTFSLPGGVRRARLYVTGLGVYEVELNGQRVGDHVLSPGWTSYRHRLLYETFDVTGLLREGDNCLGAILGDGWYRGRLGFGGGRRNLYGERLALLAQLEVELEDGSRQVVVTDGSWRAHRGPILESGIYDGEVYDARLEMPGWSTPEYDDSEWAGTRELGWPTESLEPLEVPARRTQEVAPREILRSFSGKTIVDFGQNLVGRVRLRVSGPRGQRVRLRHAEVLEGGELCTRTLRTARATDEYVLRGDGEEEWEPRFTFHGFRYVEVEGWPGELRAEDLVAVVCHSDMERIGWFGCSDPLVERLHENVVWSMRGNFLHIPTDCPQRDERLGWTGDIQVFSPAACFIYDASGFLTSWLRDVALDQDESGAVPFVVPNALGGQVIPAAAWGDAAVIVPWVLYQRYGDAGVLEAQWPSMRAWVDCIKTIAGPARLWNKGFQFGDWLDPAAPPDNPAAARTDPYIVASAYFARSAEIVGLSAQVLGMQDMAEEYLGLASEVREAFNREYVTPNGRVVSDAQTAYSLAIGFALLPTQEQRQHAGERLAELVRAEGYKIGTGFVGTPLICDALCATGHHDVAYRLLMSRECPSWLYPVTMGATTIWERWDSLRPDGSVNPGEMTSFNHYALGAVADWLHRVVGGLAPAEPGYRKLRIQPVPGGGLSYARARHVTPYGTAECSWRTEGGEIEVRVVVPPNTSAQVVLPGSGREVEVGSGEHVWRYAFEAHRYPPVTLDTPLKEILEDAEAWEVLTRHFPEVASMPPRRLERIGTIRDLAASVVAFNERVGRLERELQALSRERS

SEQ ID NO: 11 - protein, 954 aa, unidentified
  source 1..954: note = Thermomicrobia PRI-1686
MQWQASWIWLEGEPSPRNDWVCFRKSFELDRSASPLEEAKLSITADSRYVLYVNGQLVGRGPVRSWPFEQSYDTYDLRHLLHPGRNCLAVLVTHFGVSTFSYVRGRGGLLAQLELSSGDDRTTIGTDGSWKVHRHLGYSRRTTRISPQQGFVEQLDARAWSSEWKDLMYDDSGWEDAMIVGPVGTPPWEQLVPRDIPFLTEEVLHPTRVVSLHSTVPPKIAVAVDMRAIMMPDSADHAEQVQYAGFLATILRTDGEGTARLLLSKPWVGDGIAASINGQVYGAELMSRTPTGRELEVELSAGDNLLLVYVCGSDHADPLRLALDSDLGLELVSPTGGESAFVAIGPLASRVVRNFDFSQPLEYDETAVRRISSCASVADLRAWSHLPRSVPPELVSPADVFTLCTWPRQRTELTTGKELEAMVFPSKDPGLVPILRAGDTELVLDFGQEVSGYLFLDVEASEGTLIDLYGFEFMEDDYRQDTVGLDNTLRYTCREGRQHYVSPQRRGLRYLMLTVREARAPLRVHGVGVVQSTYPVSQVGTFRCSDPLLNDIWEISRLTTKLCMEDTFVDCPAYEQTFWVGDSRNEALTAYYLFGAEELVRRCLRLVPGSRRYTPLYMDQVPSGWVSVIPNWTFLWVMACREYYERTGDLAFVQDIWPDIQYTLDHYLQHINDDGLLEISAWNLLDWAPIDQPNSGVVTHQNCFFVRALKDADELGQSAGDETAGRYAERARELAAAINTHLWSDEHKAYIDSIHADSTRSSVISMQTQVVALLTGVAEGDRAEVVRSHIASPPAGWVQIGSPFMSFFLYEAMVRQGMYAQMLEDIRQKYGLMLEHGATTCWETFPGALGARYTRSHCHAWSAAPGYFLGAYVLGVRPGGPGWHRVIVAPQPCDLAWARGSVPLPRGDRVDVSWRREGQKLLLRVERPQEVELEVVPPEEYELELDERVRQTTQ

SEQ ID NO: 12 - protein, 763 aa, unidentified
  source 1..763: note = Candida molischiana
MKSTIIILSVLAAATAKNISKAEMENLEHWWSYGRSDPVYPSPEISGLGDWQFAYQRAREIVALMTNEEKTNLTFGSSGDTGCSGMISDVPDVDFPGLCLQDAGNGVRGTDMVNAYASGLHVGASWNRQLAYDRAVYMGAEFRHKGVNVLLGPVVGPIGRVATGGRNWEGFTNDPYLAGALVYETTKGIQENVIACTKHFIGNEQETNRNPSGTYNQSVSANIDDKTMHELYLWPFQDSVRAGLGSIMGSYNRVNNSYACKNSKVLNGLLKSELGFQGFVVSDWGGQHTGIASANAGLDMAMPSSTYWEEGLIEAVKNGTVDQSRLDDMATRIIAAWYKYARLDDPGFGMPVSLAEDHELVDARDPAAASTIFQGAVEGHVLVKNENALPLKKPKYISLFGYDGVSTDVNTVGGGFSFFSFDVKAIENKTLISGGGSGTNTPSYVDAPFNAFVAKAREDNTFLSWDFTSAEPVANPASDACIDFINAAASEGYDRPNLADKYSDKLVEAVASQCSNTIVVIHNAGIRLVDNWIEHENVTGVILAHLPGQDTGTSLIEVLYGNQSPSGRLPYTVAKKASDYGGLLWPTEPEGDLDLYFPQSNFTEGVYIDYKYFIQKNITPRYEFGYGLTYTTFDYSELEVDAITNQSYLPPDCTIEEGGAKSLWDIVATVKFTVTNTGDVAAAEVPQLYVGIPNGPPKVLRGFDKKLIHPGQSEEFVFELTRRDLSTWDVVAQNWGLQAGTYQFYVGRSVFDVPLTSALVFTN

SEQ ID NO: 13 - protein, 551 aa, Olea europaea
MDIQSNVLTITSGSSPTDTSSNGQAAKSTKERIKRSDFPSDFVFGAATASYQVEGAWNEGGKGMSNWDYFTQSQPGGISDFSNGTIAIDHFNMFKDDVVVMKKLGLKAYRFSLSWPRILPGGRLCHGVSKEGVQFYNDLIDALLAADIEPYITIFHWDIPQCLQLEYGGFLHERVVQDFIEYSEICFWEFGDRVKYWITLNEPWSFTVQGYVAGAFPPNRGVTPKDTEETKKHARLHRGGGKLLTAFKYGNPGTEPYKVAHNLILCHAHAVDIYRTKYQESQGGKIGITNCISWNEPLTDSQEDKDAATRGNDFMLGWFVEPVVTGEYPESMIKNVGDRLPKFSEKEEKLVKGSYDFLGINYYTSTYTSDDPTKPTTDSYLTDSRTKTSHERNKVPIGAQAGSDWLYIVPWGIYRVMVDMKKRYNDPVIYITENGVDEVNDKSKTSTEALKDDIRIHYHQEHLYYLKLAMDQGVNLKGYFIWSLFDNFEWAAGFSVRFGVMYVDYANGRYTRLPKRSAVWWRNFLTKPTAVPLKNEPEKSEDRRKRLRGST

SEQ ID NO: 14 - protein, 764 aa, Saccharomyces cerevisiae
MPAKIHISADGQFCDKDGNEIQLRGVNLDPSVKIPAKPFLSTHAPIENDTFFEDADKVSFINHPLVLDDIEQHIIRLKSLGYNTIRLPFTWESLEHAGPGQYDFDYMDYIVEVLTRINSVQQGMYIYLDPHQDVWSRFSGGSGAPLWTLYCAGFQPANFLATDAAILHNYYIDPKTGREVGKDEESYPKMVWPTNYFKLACQTMFTLFFGGKQYAPKCTINGENIQDYLQGRFNDAIMTLCARIKEKAPELFESNCIIGLESMNEPNCGYIGETNLDVIPKERNLKLGKTPTAFQSFMLGEGIECTIDQYKRTFFGFSKGKPCTINPKGKKAWLSAEERDAIDAKYNWERNPEWKPDTCIWKLHGVWEIQNGKRPVLLKPNYFSQPDATVFINNHFVDYYTGIYNKFREFDQELFIIIQPPVMKPPPNLQNSKILDNRTICACHFYDGMTLMYKTWNKRIGIDTYGLVNKKYSNPAFAVVLGENNIRKCIRKQLSEMQKDAKSMLGKKVPVFFTEIGIPFDMDDKKAYITNDYSSQTAALDALGFALEGSNLSYTLWCYCSINSHIWGDNWNNEDFSIWSPDDKPLYHDTRAKTPTPEPSPASTVASVSTSTSKSGSSQPPSFIKPDNHLDLDSPSCTLKSDLSGFRALDAIMRPFPIQIHGRFEFAEFNLCNKSYLLKLVGKTTPEQITVPTYIFIPRHHFTPSRLSIRSSSGHYTYNTDYQVLEWFHEPGHQFIEICAKSKSRPNTPGSDTSNDLPAECVIS

SEQ ID NO: 15 - protein, 861 aa, unidentified
  source 1..861: note = Thermoascus aurantiacus IFO9748
MRLGWLELAVAAAATVASAKDDLAYSPPFYPSPWMDGNGEWAEAYRRAVDFVSQLTLAEKVNLTTGVGWMQEKCVGETGSIPRLGFRGLCLQDSPLGVRFADYVSAFPAGVNVAATWDKNLAYLRGKAMGEEHRGKGVDVQLGPVAGPLGRHPDGGRNWEGFSPDPVLTGVLMAETIKGIQDAGVIACAKHFIGNEMEHFRQASEAVGYGFDITESVSSNIDDKTLHELYLWPFADAVRAGVGSFMCSYNQVNNSYSCSNSYLLNKLLKSELDFQGFVMSDWGAHHSGVGAALAGLDMSMPGDTAFGTGKSFWGTNLTIAVLNGTVPEWRVDDMAVRIMAAFYKVGRDRYQVPVNFDSWTKDEYGYEHALVGQNYVKVNDKVDVRADHADIIRQIGSASVVLLKNDGGLPLTGYEKFTGVFGEDAGSNRWGADGCSDRGCDNGTLAMGWGSGTADFPYLVTPEQAIQNEILSKGKGLVSAVTDNGALDQMEQVASQASVSIVFVNADSGEGYINVDGNEGDRKNLTLWKGGEEVIKTVAANCNNTIVVMHTVGPVLIDEWYDNPNVTAIVWAGLPGQESGNSLVDVLYGRVSPGGKTPFTWGKTRESYGAPLLTKPNNGKGAPQDDFTEGVFIDYRRFDKYNETPIYEFGFGLSYTTFEYSDIYVQPLNARPYTPASGSTKAAPTFGNISTDYADYLYPEDIHKVPLYIYPWLNTTDPKKSSGDPDYGMKAEDYIPSGATDGSPQPILPAGGAPGGNPGLYDEMYRVSAIITNTGNVVGDEVPQLYVSLGGPDDPKVVLRNFDRITLHPGQQTMWTTTLTRRDISNWDPASQNWVVTKYPKTVYIGSSSRKLHLQAPLPPY

SEQ ID NO: 16 - protein, 856 aa, unidentified
  source 1..856: note = Aspergillus oryzae
MLTSPTARTSVRISRPATTERPNTVLTSGSLDIAMVQVVSRTLTPPTSNMKLSAALSTLAALQPAVGAAVQNRASDVADLEHYWSYGHSEPVYPTPETKGLGDWEEAFTKARSLVAQMTDKEKNNITYGYSSTANGCGGTSGGVPRLGFPGICLQDAGNGVRGTDMVNSYASGVHVGASWNRDLTYSRAQYMGAEFKRKGVNVALGPVAGPIGRIARGGRNWEGFSNDPYLSGALTGDTVRGLQESVIACVKHLIGNEQETHRSTPSMLANSRNQSSSSNLDDKTMHELYLWPFQDAVKAGAGSVMCSYNRINNSYGCQNSKAMNGLLKGELGFQGFVVSDWGAQHTGIASAAAGLDMAMPSSSYWENGTLALAVKNESLPSTRLDDMATRIVATWYKYAEIENPGHGLPYSLLAPHNLTDARDPKSKSTILQGAVEGHVLVKNTNNALPLKKPQFLSLFGYDAVAAARNTMDDLDWNMWSMGYDNSLTYPNGSAVDAMMLKYIFLSSANPSAFGPGVALNATTITGGGSGASTASYIDAPFNAFQRQAYDDDTFLAWDFASQNPLVNPASDACIVFINEQSSEGWDRPYLADPYSDTLVQNVASQCSNTMVVIHNAGVRLVDRWIENDNITAVIYAHLPGQDSGRALVEVMYGKQSPSGRLPYTVAKNESDYGSLLNPVIQSGTDDIYYPQDNFTEGVYIDYKAFVAANITPRYEFGYGLTYSTFDYSDLKVSTSSNVSTSYLAPGTTVAEGGLPSVWDIIATVTCTVSNTGSVAAAEVAQLYIGIPGGPAKVLRGFEKQLIEPGQQVQVTFDLTRRDLSTWDTEKQNWGLQAGSYALYVGKSVLDIQLTGSLSL

SEQ ID NO: 17 - protein, 860 aa, unidentified
  source 1..860: note = Aspergillus niger
MRFTSIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVSVTIKNTGKVAGDEVPQLYVSLGGPNEPKIVLRQFERITLQPSKETQWSTTLTRRDLANWNVETQDWEITSYPKMVFAGSSSRKLPLRASLPTVH

SEQ ID NO: 18 - protein, 466 aa, unidentified
  source 1..466: note = Trichoderma reesei
MLPKDFQWGFATAAYQIEGAVDQDGRGPSIWDTFCAQPGKIADGSSGVTACDSYNRTAEDIALLKSLGAKSYRFSISWSRIIPEGGRGDAVNQAGIDHYVKFVDDLLDAGITPFITLFHWDLPEGLHQRYGGLLNRTEFPLDFENYARVMFRALPKVRNWITFNEPLCSAIPGYGSGTFAPGRQSTSEPWTVGHNILVAHGRAVKAYRDDFKPASGDGQIGIVLNGDFTYPWDAADPADKEAAERRLEFFTAWFADPIYLGDYPASMRKQLGDRLPTFTPEERALVHGSNDFYGMNHYTSNYIRHRSSPASADDTVGNVDVLFTNKQGNCIGPETQSPWLRPCAAGFRDFLVWISKRYGYPPIYVTENGTSIKGESDLPKEKILEDDFRVKYYNEYIRAMVTAVELDGVNVKGYFAWSLMDNFEWADGYVTRFGVTYVDYENGQKRFPKKSAKSLKPLFDELIAAA

SEQ ID NO: 19 - protein, 744 aa, unidentified
MRYRTAAALALATGPFARADSHSTSGASAEAVVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 20 - protein, 875 aa, unidentified
  source 1..875: note = Neurospora crassa
MKFAIPLALLASGNLALAAPEPIHPSHQQLNKRSLAYSEPHYPSPWMDPKAIGWEEAYEKAKAFVSQLTLLEKVNLTTGIGWGAEQCVGQTGAIPRLGLKSMCMQDAPLAIRGTDYNSVFPAGVTTAATFDRGLMYKRGYALGQEAKGKGVTVLLGPVAGPLGRAPEGGRNWEGFSTDPVLTGIAMAETIKGTQDAGVVACAKHFIGNEQEHFRQVGESQDYGYNISETLSSNIDDKTMHEMYLWPFVDAIRAGVGSFMCAYTQANNSYSCQNSKLLNNLLKQENGFQGFVMSDWQAHHSGVASAAAGLDMSMPGDTMFNSGRSYWGTNLTLAVLNGTVPQWRIDDMAMRIMAAFFKVGQTVEDQEPINFSFWTLDTYGPLHWAARKDYQQINWHVNVQGDHGSLIREIAARGTVLLKNTGSLPLKKPKFLAVIGEDAGPNPLGPNGCADNRCNNGTLGIGWGSGTGNFPYLVTPDQALQARAVQDGSRYESVLRNHAPTEIKALVSQQDATAIVFVNANSGEGFIEIDGNKGDRLNLTLWNEGDALVKNVSSWCNNTIVVLHTPGPVLLTEWYDNPNITAILWAGMPGQESGNSITDVLYGRVNPSGRTPFTWGATRESYGTDVLYEPNNGNEAPQLDYTEGVFIDYRHFDKANASVLYEFGFGLSYTTFEYSNLKIEKHQVGEYTPTTGQTEAAPTFGNFSESVEDYVFPAAEFPYVYQFIYPYLNSTDMSASSGDAQYGQTAEEFLPPKANDGSAQPLLRSSGLHHPGGNPALYDIMYTVTADITNTGKVAGDEVPQLYVSLGGPEDPKVVLRGFDRLRVEPGEKVQFKAVLTRRDVSSWDTVKQDWVITEYAKKVYVGPSSRKLDLEEVLP

SEQ ID NO: 21 - protein, 735 aa, unidentified
  source 1..735: note = Neurospora crassa
MHLRIFAVLAATSLAWAETSEKQARQAGSGFAAWDAAYSQASTALSKLSQQDKVNIVTGVGWNKGPCVGNTPAIASIGYPQLCLQDGPLGIRFGGSVTAFTPGIQAASTWDVELIRQRGVYLGAEARGVGVHVLLGPVAGALGKIPNGGRNWEGFGPDPYLTGIAMSETIEGIQSNGVQACAKHFILNEQETNRDTISSVVDDRTMHELYLFPFADAVHSNVASVMCSYNKVNGTWACENDKIQNGLLKKELGFKGYVMSDWNAQHTTNGAANSGMDMTMPGSDFNGKTILWGPQLNTAVNNGQVSKARLDDMAKRILASWYLLEQNSGYPATNLKANVQGNHKENVRAVARDGIVLLKNDDNILPLKKPSKLAIIGSSSVVNPAGRNACTDRGCNTGALGMGWGSGTADYPYFVAPYDALKTRAQSDGTTVNLLSSDSTSGVANAASGADAALVFITADSGEGYITVEGVTGDRPNLDPWHNGNQLVQAVAQANKNTIVVVHSTGPIILETILAQPGVKAVVWAGLPSQENGNALVDVLYGLVSPSGKLPYTIAKSESDYGTAVQRGGTDLFTEGLFIDYRHFDKNGIAPRYEFGFGLSYTNFTYSSLSITSTASSGPASGDTIPGGRADLWETVATVTAVVKNTGGVQGAEAPQLYITLPSSAPSSPPKQLRGFAKLKLAPGESKTATFILRRRDLSYWDTGSQNWVVPSGSFGVVVGASSRDLRLNGKFDVY

SEQ ID NO: 22 - protein, 820 aa, unidentified
  source 1..820: note = Aspergillus oryzae
MLSPQALFGALVLLSAPSAVVADTCKAPINHPGEPFSFVQPLNTTILTPYGHSPPAYPSPNTTGNGGWETALVKAKQWVNKLTLEEKTWMATGQPGPCVGNVLPIPRLNFSGICLQNGPQCVQQGDYSSVFVSSVSAAASWDRKLLYERAYALAEEHKAKGSHVILGPIGGPLGRSPYDGRTWEGFAADPYLTGVCMEETINGMQDAGVQANAKHFIANEQETQRNPTYAPDANATTYIQDSVSANIDDRTLHEIYMWPFANAVRARVASAMCSYNRLNGSHSCQNSYLLNHLLKGELGFQGYVMSDWGATHSGVASIESGMDMTMPGGFTLYGELWTEGSFFGKNLTEAVQNGTVPMSRLDDMIVRIMTPYFWLGQEKNYPSVDASVGPLNVDSAPDTWLYDWKFTGATNRDVRANHSAMIREHGGQSTVLLKNERNALPLRKPRNIVVAGNDAGPLTQGPDLQADFEYGVLAGSSGSGSCRFSYLSTPLDAINARARKDGSLVQSYLNNTLLTTSALTSPLWIPQQPDVCLVFLKSFSAEGEDRTSLELDWNGNAVVEAVATHCNNTIVITNSGGANVMPFADHPNVTAILAQHYAGEETGNAIADVLYGDVNPSAKLPYVIAYNESDYNAPLTTAVQTNGTYDWQSWFDEELEVGYRYFDAHNISVRYEFGFGLSYTTFDLKDLKAKGSAAANLTALPAKRPTEPGGNPALWETVYTLEAEVSNTGDVDGYAVPQLYLQFPTSTPAGTPPSQLRGFDKIWLEAGEKKTVTFDLMRRDVSYWDVVAQDWRIPAGAFTFKAGFSSRDFRANSVATLVKA

SEQ ID NO: 23 - protein, 737 aa, Oenococcus oeni
MSKITSIISGLSLKEKADLVSGKDFWFTAQVSGLDRMMVSDGPSGLRKQADASDALGLNKSVVAVNFPSSSLTAASFDRALLQELGRNLGQAAKAERVGILLGPGINLKRSPLAGRNFEYFSEDPYLTGELASSYVQGVQESGVGVSLKHFAANNREDQRFTASSNIDQRSLHELYLSAFEKVVKMARPATIMCSYNAINGTLNSQNQRLLTQILREEWGFKGLVMSDWGAVSDHVAALKAGLDLEMPGKGNESTSEIIEAVNKGQLDEKVLERAASRVIQMVEKWQPENKTVISYDLEKQHRFARQLAGESIVLLKNEQQLLPLKSNQSLAVIGQLAEKPRYQGSGSAHVNAFNTTTPLKVVQDILPKTAYQAGYQIDSDQIDQQAEQAAVDLAKQADQVVVFAGFPSSYESEGFDKKTISLPDNQNHLIERLAAVNKKIIVVLENGSALEMPWVGQVEAIVETYLAGEAVGEATWDILFGRVNPSGKLAESFPIKLADNPTYLTFNADRKNENYHEGLFVGYRYYDKKKQEVLFPFGHGLSYTTFEYRKLELLKSDHEVTVSFEIKNTGSVAGKETAQIYLSNQTSEIEKPLKELKGFAKVSLNPGQTKQVEIVLDKRSFSWYNPETDKWQVDNGSYQIQLAASSRDIRLTKNLLIDWSENKRQALSPDSYLSDILKEQAFKAPLKESGLDKLLEQLAGDENNQAILTNMPLRALMMMGVSNHQIQQFIKLANQS

SEQ ID NO: 24 - protein, 737 aa, Oenococcus oeni
MSKITSIISGLSLKEKADLVSGKDFWFTAQVSGLDRMMVSDGPSGLRKQADASNALGLNKSVVAVNFPSSSLTAASFDRALLQELGRNLGQAAKAERVRILLGPGINLKRSPLAGRNFEYFSEDPYLTGELASSYVQGVQESGVGVSLKRFAANNREDQRFTASSNIDQRSLHELYLSAFEKAVKMARPATIMCSYNAINGTLNSQNQRLLTQILREEWGFKGLVMSDWGAVSDHVAALKAGLDLEMPGKGNESTSEIIEAVNKGQLDEKVLERAASRVIQMVEKWQPENKTVISYDLEKQHRFARQLVGESIVLLKNEQQLLPLKSNQSLAVIGQLAEKPRYQGSGSAHVNAFNTTTPLKVVQDILPKTAYQAGYQIDSDQIDQQAEQAAVDLAKQADQVVVFSGFPSSYESEGFDKKTISLPDNQNHLIERLAAVNKKIIVVLENGSALEMPWVGQVEAIVETYLAGEAVGEATWDILFGRVNPSGKLAESFPIKLADNPTYLTFNADRKNENYHEGLFVGYRYYDKKKQEVLFPFGHGLSYTTFEYRKLELLKSDHEVTVSFEIKNTGSVAGKETAQIYLSNQTSEIEKPLKELKGFAKVSLNPGQTKQVEIVLDKRSFSWYNPETDKWQVDNGSYQIQLAASSRDIRLTKNLLIDWSENKVQALSPDSYLSDILKEQAFKAPLKESGLDKLLEQLAGDENNQAILTNMPLRALMMMGVSNHQIQQFIKLANQS

SEQ ID NO: 25 - protein, 544 aa, Prunus dulcis
MALQFRSLLLCMVLLLLGFALANTNAARTDPPIVCATLNRTHFDTLFPGFTFGAATAAYQLEGAANIDGRGPSVWDNFTHEHPEKITDGSNGDVAIDQYHRYKEDVAIMKDMGLDAYRFSISWSRLLPNGKLSGGINKKGIEYYNNLTNELLRNGIEPLVTLFHWDVPQALVDEYGGLLSPRIVDDFKAYADLCYKEFGDRVKHWTTLNEPYTISNHGYTIGIHAPGRCSDWYNPKCLGGDSGIEPYLVTHYLLLAHAAAVKLYREKYQAYQNGVIGITVVSHWFEPASESQQDKDAAFQALDFMYGWFMDPLTRGDYPQIMRSILGARLPNFTEEQSKSLSGSYDYIGVNYYSARYASAYPKDYSVTTPPSYLTDVHVNVTTDLNGVPIGPRAASDWLYVYPKGLYDLVLYTKEKYNDPIMYITENGMDEFNNPKLSLEQALNDANRIDYYYRHLCYLQAAMKEGANVQGYFAWSLLDNFEWSEGYTVRFGINYIDYDNGLERHSKLSTHWFKSFLKRSSISKKKIRRCGNNNGRATKFVYQI

SEQ ID NO: 26 - protein, 542 aa, Prunus dulcis
MAMQLRSLLLCVLLLLLGFALADTNAAARIHPPVVCANLSRANFDTLVPGFVFGAATASYQVEGAANLDGRGPSIWDTFTHKHPEKIADGSNGDVAIDQYHRYKEDVAIMKDMGLESYRFSISWSRVLPNGTLSGGINKKGIEYYNNLINELLHNGIEPLVTLFHWDVPQTLEDEYGGFLSNRIVNDFEEYAELCFKKFGDRVKHWTTLNEPYTFSSHGYAKGTHAPGRCSAWYNQTCFGGDSATEPYLVTHNLLLAHAAAVKLYKTKYQAYQKGVIGITVVTPWFEPASEAKEDIDAVFRALDFIYGWFMDPLTRGDYPQSMRSLVGERLPNFTKKESKSLSGSFDYIGINYYSARYASASKNYSGHPSYLNDVNVDVKTELNGVPIGPQAASSWLYFYPKGLYDLLRYTKEKYNDPIIYITENGVDEFNQPNPKLSLCQLLDDSNRIYYYYHHLCYLQAAIKEGVKVKGYFAWSLLDNFEWDNGYTVRFGINYVDYDNGLKRHSKHSTHWFKSFLKKSSRNTKKIRRCGNNNTSATKFVF

SEQ ID NO: 27 - protein, 450 aa, Bacillus circulans
MSIHMFPSDFKWGVATAAYQIEGAYNEDGRGMSIWDTFAHTPGKVKNGDNGNVACDSYHRVEEDVQLLKDLGVKVYRFSISWPRVLPQGTGEVNRAGLDYYHRLVDELLANGIEPFCTLYHWDLPQALQDQGGWGSRITIDAFAEYAELMFKELGGKIKQWITFNEPWCMAFLSNYLGVHAPGNKDLQLAIDVSHHLLVAHGRAVTLFRELGISGEIGIAPNTSWAVPYRRTKEDMEACLRVNGWSGDWYLDPIYFGEYPKFMLDWYENLGYKPPIVDGDMELIHQPIDFIGINYYTSSMNRYNPGEAGGMLSSEAISMGAPKTDIGWEIYAEGLYDLLRYTADKYGNPTLYITENGACYNDGLSLDGRIHDQRRIDYLAMHLIQASRAIEDGINLKGYMEWSLMDNFEWAEGYGMRFGLVHVDYDTLVRTPKDSFYWYKGVISRGWLDL

SEQ ID NO: 28 - protein, 458 aa, unidentified
  source 1..458: note = Sinorhizobium meliloti
MMIEAKKLAARFPGDFVFGVATASFQIEGASKADGRKASIWDAFSNMPGRVYGRHNGDVACDHYNRLEQDLDLIKSLGVEAYRFSIAWPRIVPEGTGPINEKGLDFYDRLVDGLKARGIKAFATLYHWDLPLALMGDGGWTARTTAYAYQRYAKTVIARLGDRLDAVATFNEPWCSVWLGHLYGVHAPGERNMDAALAALHFTNLAHGLGVAAIRSERPELPVGIVINAHSVYPGSNSAEDKAAAERAFDFHNGVFFDPIFKGEYPEDFLSALGERMPAIEDGDMATIAQPLDWWGLNYYTPMRVSADPAKGAEYPATVNAKPVSNVKTDIGWEVYAPALGSLVETLNARYRLPDCYITENGACYNMGVENGTVDDQPRLDYISDHLAVTADLIAKGYPMRGYFAWSLMDNFEWAEGYRMRFGIVHVDYETQVRTIKKSGRWYKDLAERFPSGNHKPG

SEQ ID NO: 29 - protein, 20 aa, synthetic construct
MKLSTVLLSAGLASTTLAQS

SEQ ID NO: 30 - protein, 90 aa, synthetic construct
MRFPSIFTAVLFAASSALAAPVNTTTEDETAQIPAEAVIGYLDLEGDFDVAVLPFSNSTNNGLLFINTTIASIAAKEEGVSLDKREAEAS

SEQ ID NO: 31 - protein, 20 aa, synthetic construct
MRFPSIFTAVLFAASSALAS

SEQ ID NO: 32 - protein, 64 aa, synthetic construct
MLQSVVFFALLTFASSVSAIYSNNTVSTTTTLAPSYSLVPQETTISYADDTTTFFVTSTLDKRS

SEQ ID NO: 33 - protein, 110 aa, synthetic construct
MFNRFNKFQAAVALALLSRGALGDSYTNSTSSADLSSITSVSSASASATASDSLSSSDGTVYLPSTTISGDLTVTGKVIATEAVEVAAGGKLTLLDGEKYVFSSDLDKRS

SEQ ID NO: 34 - protein, 166 aa, synthetic construct
MQYKKTLVASALAATTLAAYAPSEPWSTLTPTATYSGGVTDYASTFGIAVQPISTTSSASSAATTASSKAKRAASQIGDGQVQAATTTASVSTKSTAAAVSQIGDGQIQATTKTTAAAVSQIGDGQIQATTKTTSAKTTAAAVSQISDGQIQATTTTLAPKLDKRS

SEQ ID NO: 35 - protein, 115 aa, synthetic construct
MFNRFNKLQAALALVLYSQSALGQYYTNSSSIASNSSTAVSSTSSGSVSISSSIELTSSTSDVSSSLTELTSSSTEVSSSIAPSTSSSEVSSSITSSGSSVSGSSSITSSLDKRS

SEQ ID NO: 36 - protein, 19 aa, synthetic construct
MLLQAFLFLLAGFAAKISA

SEQ ID NO: 37 - protein, 352 aa, Silene latifolia
MENPKELLNAQAHIWNHIFAYHSSAALKCAIELGIPDTIEKHGNPMTLQDLANSLAITPTKTLSLYRLLRLLVHSNFFSMTKLVDGEEAYANNINSQLLLKDHPCTLAPFTLGMLDPAMTEPPHYLSKWFQNQDESVFHVIHGRSFWEHAGLTPGFNQLFNRAMGSDASFVSIALVANKDFAKMVEGIGSLVDVAGGDGTVAKIIARAYPWLKCTVFDLPQVVDGLQGNGSNLEYVAGDMFKEIPSADVVMLKWILHDWSDEHCVRILERCKEAIPSNGKIIIIDMVVDPQAQNNNHFHAQLLSDMEMMALNVGGIERTEDQWKKLFLQAGFNHYNIFPILGIRSVIEVRCL

SEQ ID NO: 38 - protein, 352 aa, Solanum lycopersicum
MSSSESTSSELLHAQAQIWNYIFNFISSSAVRCAFQLGIPDVLYKHDKPMCLSDISAELSVVNSSKVSFLPILMQFLVQSGFLNQHEDHYSLTPASCLLAKDDPFNVRSLLLLNHGQAFSKAWPELSDWFQNDSPTPFHTAHGKSLWDFIGEEQPSVLGDIFNDALASDSRLNTNVLIAECKHVFEGLTSLVDVGGGTGTVSIAIAKAFPNIKCTVLDLPQVVGDLKGSGNLDFVGGDMFDMIPHTNAILLKCVLHDWNDEDCVKVLKKCKESIPSREKGGKVIIIDTVLEDPKQSNEFVRAQHNMGMLMMVLFAAKERTEKEWEKLFSEAGFTEYKIFPALGLRSLIEIYP

SEQ ID NO: 39 - protein, 360 aa, Solanum lycopersicum
METNNNVERANELFKAQAHIYKHAFAYANSMALNCAIQLGIPDIIHNHKKPITLPDLLSGLKLPSSKSNAIHRLMRLLVHAQFFDIIKLEENSETEGYVLTTSSRLLLKSEIPNLLPCVRLMVDPVLVTPWQLLGEWFHKNEEATPFETAHGMPMWDFCAQNPIFDTAFNEAMASDSQMMKLVVKDCREVFEGLNSLVDVGGGTGVIAKTILEAIPHLKCTVLDLPHVVANMPQTENLIYVGGNMFQCIPHADAILLKHVMHDWSDEDCVKILKRCREAIEDKDEGRKGKVLIIDMVLGRDEEEANMTEVKLIFDVLMMVVTTGRQRTEKEWEKLFTEAGFMSYKITPLLGLRSLIQVFP

SEQ ID NO: 40 - protein, 364 aa, Solanum lycopersicum
MGSTANIQLATQSEDEERNCTYAMQLLSSSVLPFVLHSTIQLDVFDILAKDKAATKLSALEIVSHMPNCKNPDAATMLDRMLYVLASYSLLDCSVVEEGNGVTERRYGLSRVGKFFVRDEDGASMGPLLALLQDKVFINSWFELKDAVLEGGVPFDRVHGVHAFEYPKLDPKFNDVFNQAMINHTTVVMKRILENYKGFENLKTLVDVGGGLGVNLKMITSKYPTIKGTNFDLPHVVQHAPSYPGVDHVGGDMFESVPQGDAIFMKWILHDWSDGHCLKLLKNCHKALPDNGKVIVVEANLPVKPDTDTTVVGVSQCDLIMMAQNPGGKERSEQEFRALASEAGFKGVNLICCVCNFWVMEFYK

SEQ ID NO: 41 - protein, 367 aa, unidentified
  source 1..367: note = Rosa hybrida
MERLNSFRHLNQKWSNGEHSNELLHAQAHIWNHIFSFINSMSLKSAIQLGIPDIINKHGYPMTLSELTSALPIHPTKSHSVYRLMRILVHSGFFAKKKLSKTDEEGYTLTDASQLLLKDHPLSLTPYLTAMLDPVLTNPWNYLSTWFQNDDPTPFDTAHGMTFWDYGNHQPSIAHLFNDAMASDARLVTSVIINDCKGVFEGLESLVDVGGGTGTLAKAIADAFPHIECTVLDLPHVVADLQGSKNLKYTGGDMFEAVPPADTVLLKWILHDWSDEECIKILERSRVAITGKEKKGKVIIIDMMMENQKGDEESIETQLFFDMLMMALVGGKERNEKEWAKLFTDAGFSDYKITPISGLRSLIEVYP

SEQ ID NO: 42 - protein, 366 aa, unidentified
  source 1..366: note = Rosa hybrida
MERLNSFKHLNQKWSNGEHSNELLHAQAHIWNHIFSFINSMSLKSAIQLGIPDIINKHGPMTLSELTSALPIHPTKSHSVYRLMRILVHSGFFAKKKLSKTDEEGYTLTDASQLLLKDHPLSLTPFLTAMLDPVLTTPWNYLSTWFQNEDPTPFDTAHGMTFWDYGNHQPSIAHLFNDAMASDARLVTSVIIDDCKGVFEGLESLVDVGGGTGTVAKAIADAFPHIECTVLDLPHVVADLQGSKNLKYTGGDMFEAVPPADTVLLKWILHDWNDEECIKILKRSRVAITSKDKKGKVIIIDMMMENQKGDEESIETQLFFDMLMMALVRGQERNEKEWAKLFTDAGFSDYKITPILGLRSLIEVYP

SEQ ID NO: 43 - protein, 357 aa, Ocimum basilicum
MALQKVDISLSTEQLLQAQVHVWNHMYAFANSMSLKCAIQLGIPDILHKHGRPMTLSQLLQSIPINKEKTQCFQRLMRALVNSNFFIEENNSNNQEVCYWLTPASCLLLKEAPLTVTPLVQVVLDPTFTNPWHHMSEWFTHEKHATQFEAANGCTFWEKLANEPSKGRFFDEAMSCDSRLIAHVFTKDYKHVIEGIRTLVDVGGGNGTMAKAIVEAMPTIKCTVIDLPHVVAGLESTDNLNYIGGDMFQSIPSADAILLKSIIHDWDDVEGLKILKKCKDAVVMGGKVIIIDVVVGVNHDIDEVLEDQLHFDMAMMCYFNAKERTMSEWEKLIYDAGFKSYKLTPAFGVRSLIEAYP

SEQ ID NO: 44 - protein, 387 aa, Eriobotrya japonica
MGLSNGDEVGATSHELLGAQAQIWNHMFQFINSMALKCAVQLGIPDVIHNHGQPISLSELIAALNVHPSKAHFVSRLMRILVHSDFFAQHHHVHHDCDDVEEEEAVVLYSLTPTSRLLLKDGPSNTTPLVLMILDPVLTTPFHLMGAWLQMNGGDDPATICTPFEMENGMPFWDLAAQEPRFGNLFDEAMEADSKLLGREVVEECGGVFEGLKSLVDVGGGTGTMAKAIANAFPSINCIVFDQPHVVADLQGTTHNLGFVGGDMFVEIPPANAILLKWILHDWSDEESVKILKNCREAILLSKNNEGNKKIIIIDIVVGHVDNKEKMVDKKSIETQLMFDMLMMSTVTGKERSESEWKKIFLAAGFTHYNITHAFGFRSLIELYPSK

SEQ ID NO: 45 - protein, 381 aa, Pinus taeda
MDSNMNGLAKSNGCEISRDGFFESEEEELQGQAEAWKCTFAFAESLAVKCVVLLGIPDMIAREGPRATLSLCEIVANLPTESPDAACLFRIMRFLVAKGIFPASKSARRRAFETRYGLTPASKWLVKGRELSMAPMLLMQNDETTLAPWHHFNECVLEGGVAFQKANGAEIWSYASDHPDFNNLFNNAMACNARIVMKAILSKYQGFHSLNSLVDVGGGTGTAVAEIVRAYPFIRGINYDLPHVVATASSLSGVQHVGGDMFETVPTADAIFMKWIMHDWNDEDCIKILKNCRKAIPDTGKVIIVDVVLDADQGDNTDKKRKKAVDPIVGTVFDLVMVAHSSGGKERTEKEWKRILLEGGFSRYNIIEIPALQSVIEAFPR

SEQ ID NO: 46 - protein, 365 aa, unidentified
  source 1..365: note = Fragaria ananassa
MGSTGETQMTPTHVSDEEANLFAMQLASASVLPMVLKAAIELDLLEIMAKAGPGSFLSPSDLASQLPTKNPEAPVMLDRMLRLLASYSILTCSLRTLPDGKVERLYCLGPVCKFLTKNEDGVSIAALCLMNQDKVLVESWYHLKDAVLDGGIPFNKAYGMTAFDYHGTDPRFNKVFNKGMADHSTITMKKILETYKGFEGLKSIVDVGGGTGAVVNMIVSKYPSIKGINFDLPHVIEDAPQYPGVQHVGGDMFVSVPKGNAIFMKWICHDWSDEHCIKFLKNCYAALPDDGKVILAECILPVAPDTSLATKGVVHMDVIMLAHNPGGKERTEQEFEALAKGSGFQGIRVCCDAFNTYVIEFLKKI

SEQ ID NO: 47 - protein, 266 aa, unidentified
  source 1..266: note = Schizosaccharomyces pombe
MPHMEDNGSEKEQLFLQHIQNLPQERLDAIRGHPELVLKEIDEFTYPDGSGVRMCIGDVKGGFIVGKIRERKPKIMVELGGYLGYSAILFGNEISKIPGGRYYSLEVNEDYAKIAYELVKLAGLDEIVTIMIGKACDSLVELQQKLLHKDLGFQALDMVFIDHWKDLYVPDLRVIESLNMIAPGTLLVADNIITPGAPEYHKYVNMSPEERRGYQAKVRNVNGFDFIGRWDLIYKTETKEFEGVIRNKHRKDAVDVTECVGYAKKD

SEQ ID NO: 48 - protein, 361 aa, Ocimum basilicum
MGSATNTPQINSDEEENFLFAMQLASASVLPMVLKSAIELDLLELIKKSGAGAFVSPVDLAAQLPTTNPDAHVMLDRILRLLTSYAILECRLKTLPDGGVERLYGLAPVCKFLTKNEDGVSMAPLTLMNQDKVLMESWYHLSDAVVDGGIPFNKAYGMTAFEYHGTDPRFNKVFNQGMSNHSTITMKKILETYTGFDGLKTVVDVGGGTGATLNMIVSKYPSIKGINFDLPHVIEDAPSYPGVEHVGGDMFVSVPKGDAIFMKWICHDWSDEHCVKFLKNCYDALPQNGKVILAECVLPEAPDTGLATKNVVHIDVIMLAHNPGGKERTEKEFQGLAKAAGFKQFNKACCAYNTWIMELLK

SEQ ID NO: 49 - protein, 351 aa, unidentified
  source 1..351: note = Zinnia elegans
MGSNQDDQAFLFAMQLASASVLPMVLKTAIELDLLETIAKAGPHGSVSSSELVAQLPKVNNPEAPVMIDRICSLLASYSVLTCTLKETADGCAERFYGLAPVCKFLIKNDAGVSLAPLLLMNQDKVLMESWYYLKDPVLDGGIPFNKAYGMSAFEYHGKDQRFNKVFNSGMFNHSTMTMKKIVELYNGFSGLKTLVDVGGGTGASLNMITSKHKSLKGINFDLPHVIADATTYQGIEHVGGDMFESVPKGDAIFMKWILHDWSDAHCLQVLKNCYKSLPENGKVIVAECILPEAPDTTPATQNVIHIDVIMLAHNPGGKERTEKEFEALAKGAGFKGFNKAACALNTWVME

SEQ ID NO: 50 - protein, 19 aa, synthetic construct
MLQSVVFFALLTFASSVSA

SEQ ID NO: 51 - protein, 26 aa, synthetic construct
MSALFNFRSLLQVILLLICSCSYVHG

SEQ ID NO: 52 - protein, 90 aa, synthetic construct
MRFPSIFTDVLFAASSALATPVNTTTEDETAQIPAEAVIGYLDLEGDFDVAVLPFSNSTNNGLLFINTTIASIAAKEEGVSLDKREAEAS

SEQ ID NO: 53 - protein, 90 aa, synthetic construct
MRFPSIFTDVLFAASSALATPVNTTTEDETAQIPAEAVIGYSDLEGDFDVAVLPFSNSTNNGLLFINTTIASIAAKEEGVSLDKREAEAS

SEQ ID NO: 54 - protein, 20 aa, synthetic construct
MRFPSIFTDVLFAASSALAT